(19) United States
(12) Patent Application Publication
(10) Pub. No.: US 2025/0009292 A1
(43) Pub. Date: Jan. 9, 2025

(54) ANESTHESIA MONITORING SYSTEM

(71) Applicant: ICHILOV TECH LTD., Tel-Aviv (IL)

(72) Inventor: Zvi IZAKSON MASIE, Tel-Aviv (IL)

(21) Appl. No.: 18/707,597

(22) PCT Filed: Nov. 9, 2022
(86) PCT No.: PCT/IL2022/051194

§ 371 Date: May 5, 2024

Related U.S. Application Data
(60) Provisional application No. 63/277,268, filed on Nov. 9, 2021.

(51) Int. Cl. A61B 5/00

(52) U.S. Cl. CPC A61B 5/4821; A61B 5/6814; A61B 5/6822; A61B 5/746

(57) ABSTRACT

A method for determining an effect of anesthesia in a subject, including:

stimulating a body of a subject at one or more stimulation sites, wherein the subject is under regional anesthesia;

measuring a response of the subject to the stimulation, wherein said response passes through a nervous system of the subject;

determining an effect of the regional anesthesia on the subject body based on results of the measuring.

### RELATED APPLICATION/S

This application claims the benefit of priority under 35 USC § 119 (e) of U.S. Provisional Patent Application No. 63/277,268 filed Nov. 9, 2021, the contents of which are incorporated herein by reference in their entirety.

### FIELD AND BACKGROUND OF THE INVENTION

The present invention, in some embodiments thereof, relates to monitoring and/or adjustment of anesthesia and, more particularly, but not exclusively, to monitoring and/or adjustment of regional anesthesia, for example neuraxial anesthesia.

### SUMMARY OF THE INVENTION

Some examples of some embodiments of the invention are listed below (some examples of the invention are described herein and an embodiment may include features from more than one example and/or fewer than all features of an example):
Example 1. A method for determining an effect of anesthesia in a subject, comprising: stimulating a body of a subject at one or more stimulation sites, wherein the subject is under regional anesthesia;
   measuring a response of the subject to the stimulation, wherein the response passes through a nervous system of the subject;
   determining an effect of the regional anesthesia on the subject body based on results of the measuring.
Example 2. A method according to example 1, wherein the determining the regional anesthesia effect comprises determining axial distribution of the regional anesthesia indicating anesthesia height, and/or determining depth of the regional anesthesia at one or more target regions in the body associated with the one or more stimulation sites and/or at one or more target regions located at a distance from the one or more stimulation sites.
Example 3. A method according to any one of examples 1 or 2, wherein the determining the regional anesthesia effect comprises determining axial distribution of the regional anesthesia indicating anesthesia height, and/or determining depth of the regional anesthesia at one or more target regions in the body located at a distance from the one or more stimulation sites, or one or more target regions in the body located between two or more stimulation sites.
Example 4. A method according to any one of the previous examples, comprising repeating the stimulating at two or more axially spaced-apart stimulation sites, and wherein the determining comprises determining the regional anesthesia effect of one or more target body regions located between the two or more axially spaced-apart stimulation sites.
Example 5. A method according to example 4, wherein an interval between two consecutive stimulations of the repeated stimulations is higher than a synaptic fatigue duration.
Example 6. A method according to any one of the previous examples, wherein the measuring comprises measuring the response of the subject up to 300 milliseconds following the stimulation.
Example 7. A method according to any one of the previous examples, wherein the measuring comprises measuring at least one EMG signal by at least one sensing electrode positioned at one or more EMG measurements sites on the subject body, and wherein the determining comprises determining an effect of the regional anesthesia based on the measured at least one EMG signal.
Example 8. A method according to example 7, wherein the one or more EMG sensing sites comprise at least one of facial muscle regions, back muscle regions and/or neck regions.
Example 9. A method according to any one of the previous examples, wherein the measuring comprises measuring somatosensory evoked potentials (SSEP) by at least one sensing electrode positioned at one or more SSEP measurements sites.
Example 10. A method according to example 9, wherein the one or more SSEP measurement sites comprise one or more locations on a the subject body above cortical and/or sub-cortical regions.
Example 11. A method according to any one of examples 9 or 10, wherein the one or more SSEP measurement sites comprise one or more locations at a nape of the subject, behind an car of the subject, above a mastoid, behind an car helix, on the subject body above cervical locations, and/or on a back of the subject.
Example 12. A method according to any one of the previous examples, wherein the one or more stimulation sites comprise at least one stimulation site in one or more dermatomes located between S5 to T2 dermatomes.
Example 13. A method according to any one of the previous examples, wherein said stimulating comprises delivering at least one of electric stimulation, thermal stimulation, pressure stimulation and/or tactile stimulation to the subject body at the one or more stimulation sites.
Example 14. A method according to any one of the previous examples, wherein the stimulating comprises delivering an electric field to the subject body at the one or more stimulation sites by at least one stimulating electrode, and wherein the measuring comprises measuring the response of the subject to the delivered electric field by at least one sensing electrode.
Example 15. A method according to example 14, wherein the delivered electric field has an intensity value in a range between 0.5-40 mA.
Example 16. A method according to example 14, wherein an intensity of the delivered electric field is up to 40 mA.
Example 17. A method according to any one of examples 14 to 16, wherein the delivered electric field has a frequency value in a range between 1-4000 Hz.
Example 18. A method according to any one of the previous examples, comprising determining a relation between the measured response and one or more indications stored in a memory, and wherein the regional anesthesia effect is determined based on the determined relation.
Example 19. A method according to example 18, wherein the one or more stored indications comprise at least one indication of at least one response, previously measured from the subject and/or at least one indication of measurements, previously measured from different subjects.
Example 20. A method according to any one of the previous examples, comprising administering prior to the stimulating, one or more anesthetic drugs to regions surrounding nerves of the central nervous system, through one or more administration sites, to initiate the regional anesthesia.
Example 21. A method according to example 20, wherein the stimulating comprises stimulating the subject body before and during the anesthetizing, wherein the measuring comprises measuring a response of the subject before and during the anesthetizing, and wherein the determining comprises determining the effect based on a change in a body response to a stimulation measured during the anesthetizing relative to a body response to a stimulation measured before the anesthetizing, and/or relative to an indication stored in a memory.
Example 22. A method according to any one of examples 20 or 21, comprising modifying at least one parameter of the administering of the one or more anesthetic drugs according to the determined effect.
Example 23. A method according to example 22, wherein the at least one parameter comprises anesthesia delivery rate of the one or more anesthetic drugs, dosage of the one or more anesthetic drugs, type and/or mixture ratio between the one or more anesthetic drugs, and/or an administration site of the one or more anesthetic drugs.
Example 24. A method according to any one of examples 22 or 23, wherein the modifying comprises stopping the administering.
Example 25. A method according to any one of the previous examples, comprising: detecting that the determined effect of the regional anesthesia is not according to a planned anesthesia effect; and generating an alert signal indicating a relation between the determined regional anesthesia effect and the planned anesthesia effect.
Example 26. A method according to example 25, wherein the detecting comprises detecting that a regional anesthesia depth determined based on the determined regional anesthesia effect is not according to a planned regional anesthesia depth, and wherein the generated alert signal indicates a relation between the determined regional anesthesia depth and the planned regional anesthesia depth.
Example 27. A method according to any one of examples 25 or 26, wherein the detecting comprises detecting that an axial distribution of the regional anesthesia, determined based on the determined regional anesthesia effect is not according to a planned regional anesthesia axial distribution, and wherein the generated alert signal indicates a relation between the determined axial distribution and the planned axial distribution of the regional anesthesia.
Example 28. A method according to any one of examples 25 to 27, wherein the detecting comprises detecting hemiparesis in the subject based on the determined regional anesthesia effect, and wherein the generated alert signal indicates the detected hemiparesis.
Example 29. A method according to any one of the previous examples, comprising receiving at least one signal indicating the response of the subject response to the stimulation, and wherein the measuring comprising analyzing the received at least one signal using one or more machine learning algorithms, and wherein the determining comprises determining the regional anesthesia effect based on results of the analysis.
Example 30. A method according to example 29, wherein the machine learning algorithm is configured to categorize portions of the received at least one signal into at least two groups comprising a first group of signals indicating a positive transmission of sensory information, and a second group of signals indicating a block in transmission of sensory information.
Example 31. A system for monitoring anesthesia effect on a body of a subject, comprising:
   at least one stimulator configured to deliver stimulation to at least one stimulation site on a subject body;
   at least one sensing electrode configured to sense muscle activity and/or neural activity in at least one sensing site on a subject body;
   memory;
   a control circuitry operationally connected to the at least one stimulator and the at least one sensing electrode;
   wherein the control circuitry is configured to:
   activate the at least one stimulator to deliver a stimulation to the subject body via the at least one stimulation site, according to stimulation parameters values stored in the memory, by the at least one stimulator;
   receive at least one signal from the at least one sensing electrode following the stimulation delivery;
   measure a response of the subject body to the stimulation based on the received signal; and determine an effect of anesthesia on the subject body based on the measured response, and at least one indication stored in the memory.
Example 32. A system according to example 31 wherein the anesthesia effect determined by the control circuitry comprises at least one of, axial distribution of an anesthesia effect in a subject body and/or depth of anesthesia at one or more target locations.
Example 33. A system according to example 32, wherein the at least one stimulator comprises at least one stimulating electrode shaped and sized to be positioned at the at least one stimulation site on a subject body, wherein the system further comprises at least one pulse generator functionally connected to the at least one stimulating electrode, and wherein the control circuitry is configured to:
   activate the pulse generator to generate and deliver an electric field to the at least one stimulating electrode, wherein the electric field is generated according to electric field parameter values stored in the memory;
   receive the at least one signal from the at least one sensing electrode following the electric field delivery;
   measure a response of the subject body to the delivered electric fields based on signals received from the at least one sensing electrode following the electric field delivery; and
   determine the effect of the anesthesia on the subject body based on the measured response and the at least one indication stored in the memory.
Example 34. A system according to example 33, wherein the control circuitry is configured to receive the at least one signal up to 300 milliseconds following the delivery of the electric field to the subject body.
Example 35. A system according to any one of examples 33 or 34, wherein the at least one sensing electrode comprises at least one EMG recording electrode.
Example 36. A system according to any one of examples 33 to 35, wherein the at least one sensing electrode is an electrode configured to record at least one signal related to neural activity at the one or more sensing sites, and wherein the control circuitry is configured to measure SSEP based on the neural activity related signal, and to determine an effect of anesthesia on the subject body based on the measured SSEP.
Example 37. A system according to any one of examples 33 to 36, wherein the control circuitry determines an effect of the anesthesia on the subject body by determining a relation between the measured response and one or more indications stored in the memory.
Example 38. A system according to any one of examples 33 to 37, wherein the control circuitry determines an effect of the anesthesia by activating the at least one pulse generator to generate and deliver two or more electric fields separated in time and/or in a stimulation location to the subject, by measuring a response of the subject body to the two or more electric fields, and by determining a relation between a first measured body response to a first electric field delivery, and a second body response to a second electric field delivery.
Example 39. A system according to example 38, wherein the control circuitry activates the pulse generator to generate and deliver two consecutive electric fields with an interval between the two consecutive electric field which is higher than 180 microseconds.
Example 40. A system according to any one of examples 33 to 39, wherein an intensity of the generated electric field is in a range between 0.5 mA-40 mA.
Example 41. A system according to any one of examples 33 to 39, wherein an intensity of the generated electric field is up to 40 mA.
Example 42. A system according to any one of examples 33 to 41, wherein a frequency of the generated electric field is in a range between 0.1-4000 Hz.
Example 43. A system according to any one of examples 33 to 42, comprising at least one user interface operationally connected to the control circuitry and configured to generate and deliver at least one human detectable indication to a user of the system and/or to an expert according to the determined anesthesia effect.
Example 44. A system according to example 43, wherein the at least one human detectable indication comprises an alert signal, and wherein the control circuitry signals the user interface to generate the alert signal if the determined anesthesia effect comprises a determined anesthesia depth that is not according to a planned anesthesia depth or indication thereof stored in the memory.
Example 45. A system according to example 43, wherein the at least one human detectable indication comprises an alert signal, and wherein the control circuitry signals the user interface to generate the alert signal if the determined anesthesia effect comprises a determined axial distribution of the anesthesia effect that is not according to a planned axial distribution or an indication thereof stored in the memory.
Example 46. A system according to example 43, wherein the control circuitry signals the user interface to generate the at least one human detectable indication with instructions to modify at least one parameter of the anesthesia according to the determined anesthesia effect.
Example 47. A system according to example 46, wherein the at least one parameter of the anesthesia comprises at least one of, administration site of one or more anesthetic compounds, dosage of the one or more anesthetic compounds, infusion rate of the one or more anesthetic compounds, ratio between two or more anesthetic compounds, and/or type of one or more anesthetic compounds.
Example 48. A system according to any one of examples 43 to 47, wherein the human detectable indication comprises a graphical representation of a distribution of the anesthesia effect and/or a graphical representation of a depth of the anesthesia in one or more body regions.
Example 49. A system according to any one of examples 43 to 48, wherein the control circuitry generates a pharmacodynamic profile of one or more anesthetic compounds used for the anesthesia in the subject, based on the determined anesthesia effect and/or one or more subject-related indications stored in the memory.
Example 50. A system according to example 49, wherein the subject-related indications comprise one or more indications related to a clinical state of the subject, age, gender, BMI, medical history, and/or drug regime.
Example 51. A system according to any one of examples 49 or 50, wherein the control circuitry signals the user interface to generate a human detectable indication with instructions how to modify at least one parameter of the anesthesia according to the generated pharmacodynamic profile, wherein the at least one parameter of the anesthesia comprises at least one of type of anesthetic compounds, dose, infusion rate, and/or ratio between anesthetic compounds.
Example 52. A system according to any one of examples 33 to 51, comprising a communication circuitry operationally connected to the control circuitry and the memory; wherein the control circuitry signals the communication circuitry to transmit an indication to a remote device based on information stored in the memory.
Example 53. A system according to example 52 wherein the remote device comprises a remote computer, a remote display, a cloud storage, a remote server, a remote database.
Example 54. A system according to any one of examples 33 to 53, wherein the control circuitry repeats the activate the at least one stimulator, the receive the at least one signal, the measure a response and the determine an effect every time period of up to 1 minute, and at least 5 times during an overall time period that lasts at least 5 minutes.
Example 55. A system according to any one of examples 33 to 54, comprising an electrode patch having a surface configured to attach the electrode patch to a skin surface of the subject, wherein the electrode patch comprises the at least one stimulating electrode.
Example 56. A system according to example 55, wherein the at least one stimulating electrode comprises two or more stimulating arranged as an array in the electrode patch, and wherein each of the two or more stimulating electrodes in the array is separately electrically connected to the pulse generator.
Example 57. A system according to example 56, wherein a distance between two adjacent stimulating electrodes of the at least two stimulating electrodes is at least a distance between two adjacent dermatomes on a body of a subject or is at least a distance between two adjacent vertebra on a back of a subject.
Example 58. A system according to any one of examples 56 or 57, wherein the array comprises at least one alignment marking for aligning the array and/or at least one electrode of the array with an anesthetics injection site or with an anatomical feature of a subject body.
Example 59. A system according to any one of examples 55 to 58, wherein the electrode patch comprises or is electrically connected to the at least one sensing electrode.
Example 60. A system according to any one of examples 33 to 59, comprising at least one actuator operationally connected to the control circuitry, wherein the actuator is configured to control an infusion rate of one or more anesthetic compounds into the subject body, wherein the control circuitry adjusts the activation of the actuator according to the determined anesthesia effect.
Example 61. A system according to example 60, wherein the control circuitry signals the actuator to stop or to reduce rate flow of one or more anesthetic compounds into the subject body if the determined anesthesia effect indicates distribution of the anesthesia effect towards unwanted body regions.
Example 62. A system according to any one of examples 60 or 61, wherein the actuator comprises an infusion pump.
Example 63. A system according to any one of examples 33 to 62 wherein the anesthesia comprises regional anesthesia.
Example 64. A system according to any one of examples 33 to 63, wherein the control circuitry is configured to measure the subject body response by analyzing the received at least one signal using at least one machine learning algorithm stored in the memory, wherein the machine learning algorithm is configured to categorize the received at least one signal into at least two groups comprising a first group of signals indicating sensory information transmission, and a second group of signals indicating a block in sensory information transmission, and wherein the anesthesia effect is determined based on the analysis results.
Example 65. A method for determining a neural transmission related clinical state of a subject, comprising:
   stimulating a body of a subject at one or more stimulation sites;
   measuring a response of the subject to the stimulation, wherein the response passes through a nervous system of the subject;
   determining a clinical state and/or a stage of a clinical state of the subject based on the measured response, wherein the clinical state is related to neural transmission in the subject between two or more locations in a body of the subject.
Example 66. A method according to example 65, wherein the measuring comprises measuring at least one EMG signal by at least one sensing electrode positioned at one or more EMG measurements sites on the subject body, and wherein the determining comprises determining the clinical state and/or the stage of a clinical state based on the measured at least one EMG signal.
Example 67. A method according to example 66, wherein the one or more EMG sensing sites comprise at least one of facial muscle regions, back muscle regions, limb muscle regions and/or neck regions.
Example 68. A method according to any one of examples 65 to 67, wherein the measuring comprises measuring somatosensory evoked potentials (SSEP) by at least one sensing electrode positioned at one or more SSEP measurements sites.
Example 69. A method according to example 68, wherein the one or more SSEP measurement sites comprise one or more locations on a the subject body onto cortical and/or onto sub-cortical regions.
Example 70. A method according to any one of examples 68 or 69, wherein the one or more SSEP measurement sites comprise one or more locations at a nape of the subject, behind an ear helix of the subject between the ear and the nape, on the subject body above cervical locations, and/or on a back of the subject.
Example 71. A method according to any one of examples 65 to 70, wherein the clinical state comprises peripheral neuropathy wherein said one or more stimulation sites comprise at least one stimulation site positioned on a limb of said subject.
Example 72. A method according to example 71, wherein said stimulating comprises delivering an electric field to a stimulation site on a limb of the subject, wherein the measuring comprises measuring a response signal following the electric field delivery, and wherein the determining comprises determining the peripheral neuropathy and/or a stage of the peripheral neuropathy based on a relation between the measured response signal and at least one indication stored in a memory.
Example 73. A method according to example 71, wherein the stimulating comprises delivering a first electric field to a first stimulation site on a limb of the subject, and a second electric field to a second stimulation site on a limb of the subject, wherein the measuring comprises measuring a first response signal following delivery of the first electric field, and a second response signal following delivery of the second electric field, and wherein the determining comprises determining the peripheral neuropathy and/or a stage of the peripheral neuropathy based on a difference between the first response signal and the second response signal.
Example 74. A method for determining an effect of local anesthesia in a subject, comprising:
   administering one or more anesthetic compounds at one or more administration sites, wherein the one or more anesthetic compounds are suitable for locally anesthetizing a target body region in the subject;
   stimulating the target body region of the subject at one or more stimulation sites within the target body region;
   measuring a response of the subject to the stimulation, wherein the response passes through a nervous system of the subject;
   determining an effect of the local anesthesia on the target body region based on results of the measuring.

Below are some additional examples of some embodiments of the invention (some examples of the invention are described herein and an embodiment may include features from more than one example and/or fewer than all features of an example):
Example 1. A method for determining an effect of anesthesia in a subject, comprising:
   stimulating a body of a subject at one or more stimulation sites, wherein said subject is under regional anesthesia;
   measuring a response of said subject to said stimulation, wherein said response passes through a nervous system of said subject;
   determining an effect of said regional anesthesia on said subject body based on results of said measuring
Example 2. A method according to example 1, wherein said determining said regional anesthesia effect comprises determining axial distribution of said regional anesthesia indicating anesthesia height, and/or determining depth of said regional anesthesia at one or more target regions in said body associated with said one or more stimulation sites and/or at one or more target regions located at a distance from said one or more stimulation sites.
Example 3. A method according to any one of examples 1 or 2, wherein said determining said regional anesthesia effect comprises determining axial distribution of said regional anesthesia indicating anesthesia height, and/or determining depth of said regional anesthesia at one or more target regions in said body located at a distance from said one or more stimulation sites, or one or more target regions in said body located between two or more stimulation sites.
Example 4. A method according to any one of the previous examples, comprising repeating said stimulating at two or more axially spaced-apart stimulation sites, and wherein said determining comprises determining said regional anesthesia effect of one or more target body regions located between said two or more axially spaced-apart stimulation sites.
Example 5. A method according to example 1, comprising: generating a trend and/or a prediction of the regional anesthesia effect in said subject based on said determined effect and one or more indications stored in a memory.
Example 6. A method according to example 5, wherein said one or more stored indications comprise at least one of, previous measurements of the response of said subject or a population of individuals, medical history of said subject or a population of individuals, clinical state of said subject or a population of individuals, type and/or dose of anesthetic drugs delivered to the subject or to a population of individuals.
Example 7. A method according to any one of examples 5 or 6, comprises delivering a human detectable indication with information regarding said generated trend and/or said generated prediction.
Example 8. A method according to any one of the previous examples, comprising administering prior to said stimulating, one or more anesthetic drugs to regions surrounding nerves of the central nervous system, through one or more administration sites, to initiate said regional anesthesia.
Example 9. A method according to example 8, wherein said stimulating comprises stimulating said subject body before and during said anesthetizing, wherein said measuring comprises measuring a response of said subject before and during said anesthetizing, and wherein said determining comprises determining said effect based on a change in a body response to a stimulation measured during said anesthetizing, relative to a previously measured body response to a stimulation, and/or relative to an indication stored in a memory.
Example 10. A method according to any one of the previous examples, comprising repeating said stimulating, said measuring, and said determining by a device.
Example 11. A method according to any one of the previous examples, comprising: modifying at least one parameter of a stimulation delivered to said subject body during said stimulating, and/or at least one parameter of said measuring of said response, and/or at least one parameter of delivery of said regional anesthesia, according to said determined effect.
Example 12. A method according to example 11, wherein said modifying at least one parameter of delivery of said regional anesthesia comprises modifying at least one parameter of administering of one or more anesthetic drugs according to said determined effect.
Example 13. A method according to example 12, wherein said at least one administering parameter comprises anesthesia delivery rate of said one or more anesthetic drugs, dosage of said one or more anesthetic drugs, type and/or mixture ratio between said one or more anesthetic drugs, and/or an administration site of said one or more anesthetic drugs.
Example 14. A method according to any one of examples 12 or 13, wherein said modifying at least one parameter of said administering comprises stopping said administering.
Example 15. A method according to any one of examples 11 to 14, wherein said at least one stimulation parameter comprises at least one of, stimulation intensity, stimulation frequency, stimulation duration and/or stimulation location.
Example 16. A method according to any one of examples 11 to 15, wherein said at least one parameter of said measuring comprises at least one of, type of an electrode used for said measuring, location of said measuring, processing method or algorithm used for processing of signals received during said measuring.
Example 17. A method according to any one of the previous examples, wherein said measuring comprises measuring said response of said subject up to 300 milliseconds following said stimulation.
Example 18. A method according to any one of the previous examples, wherein said measuring comprises measuring at least one EMG signal by at least one sensing electrode positioned at one or more EMG measurements sites on said subject body, and wherein said determining comprises determining an effect of said regional anesthesia based on said measured at least one EMG signal.
Example 19. A method according to example 17, wherein said one or more EMG sensing sites comprise at least one of facial muscle regions, back muscle regions and/or neck regions.
Example 20. A method according to any one of the previous examples, wherein said measuring comprises measuring event-related potentials (ERP) by at least one sensing electrode positioned at one or more ERP measurements sites.
Example 21. A method according to example 20, wherein said one or more ERP measurement sites comprise one or more locations on a said subject body above cortical and/or sub-cortical regions.
Example 22. A method according to any one of examples 20 or 21, wherein said one or more ERP measurement sites comprise one or more locations at a nape of said subject, behind an ear of said subject, above a mastoid, behind an ear helix, on said subject body above cervical locations, and/or on a back of said subject.
Example 23. A method according to any one of examples 20 to 22, wherein said ERP comprises somatosensory evoked potentials (SSEP) or electroencephalography (EEG).
Example 24. A method according to any one of the previous examples, wherein said one or more stimulation sites comprise at least one stimulation site in one or more dermatomes located between S5 to T2 dermatomes.
Example 25. A method according to any one of the previous examples, wherein said stimulating comprises delivering an electric field to said subject body at said one or more stimulation sites by at least one stimulating electrode, and wherein said measuring comprises measuring said response of said subject to said delivered electric field by at least one sensing electrode.
Example 26. A method according to example 25, wherein said delivered electric field has an intensity value in a range between 0.5-40 mA.
Example 27. A method according to example 25, wherein an intensity of said delivered electric field is up to 40 mA.
Example 28. A method according to any one of examples 25 to 27, wherein said delivered electric field has a frequency value in a range between 1-4000 Hz.
Example 29. A method according to any one of the previous examples, comprising: detecting that said determined effect of said regional anesthesia is not according to a planned anesthesia effect; and generating an alert signal indicating a relation between said determined regional anesthesia effect and said planned anesthesia effect.
Example 30. A method according to example 29, wherein said detecting comprises detecting that a regional anesthesia depth determined based on said determined regional anesthesia effect is not according to a planned regional anesthesia depth, and wherein said generated alert signal indicates a relation between said determined regional anesthesia depth and said planned regional anesthesia depth.
Example 31. A method according to any one of examples 29 or 30, wherein said detecting comprises detecting that an axial distribution of said regional anesthesia, determined based on said determined regional anesthesia effect is not according to a planned regional anesthesia axial distribution, and wherein said generated alert signal indicates a relation between said determined axial distribution and said planned axial distribution of said regional anesthesia.
Example 32. A method according to any one of examples 29 to 31, wherein said detecting comprises detecting hemiparesis in said subject based on said determined regional anesthesia effect, and wherein said generated alert signal indicates said detected hemiparesis.
Example 33. A method according to any one of the previous examples, comprising receiving at least one signal indicating said response of said subject response to said stimulation, and wherein said measuring comprising analyzing said received at least one signal using one or more machine algorithms comprising at least one of, machine learning algorithms, algorithmic classifiers, classifying models, and wherein said determining comprises determining said regional anesthesia effect based on results of said analysis.
Example 34. A system for monitoring anesthesia effect on a body of a subject, comprising:
   at least one stimulator configured to deliver stimulation to at least one stimulation site on a subject body;
   at least one sensing electrode configured to sense muscle activity and/or neural activity in at least one sensing site on a subject body;
   memory;
   a control circuitry operationally connected to said at least one stimulator and said at least one sensing electrode;
   wherein said control circuitry is configured to:
   activate said at least one stimulator to deliver a stimulation to said subject body via said at least one stimulation site, according to stimulation parameters values stored in said memory, by said at least one stimulator;
   receive at least one signal from said at least one sensing electrode following said stimulation delivery;
   measure a response of said subject body to said stimulation based on said received signal; and determine an effect of anesthesia on said subject body based on said measured response, and at least one indication stored in said memory.
Example 35. A system according to example 34 wherein said anesthesia effect determined by said control circuitry comprises at least one of, axial distribution of an anesthesia effect in a subject body and/or depth of anesthesia at one or more target locations.
Example 36. A system according to example 35, wherein said at least one stimulator comprises at least one stimulating electrode shaped and sized to be positioned at said at least one stimulation site on a subject body, wherein said system further comprises at least one pulse generator functionally connected to said at least one stimulating electrode, and wherein said control circuitry is configured to:
   activate said pulse generator to generate and deliver an electric field to said at least one stimulating electrode, wherein said electric field is generated according to electric field parameter values stored in said memory;
   receive said at least one signal from said at least one sensing electrode following said electric field delivery;
   measure a response of said subject body to said delivered electric fields based on signals received from said at least one sensing electrode following said electric field delivery; and
   determine said effect of said anesthesia on said subject body based on said measured response and said at least one indication stored in said memory.
Example 37. A system according to example 36, wherein said at least one sensing electrode is an electrode configured to record at least one signal related to neural activity at said one or more sensing sites, and wherein said control circuitry is configured to measure ERP based on said neural activity related signal, and to determine an effect of anesthesia on said subject body based on said measured ERP.
Example 38. A system according to any one of examples 36 or 37, wherein said control circuitry determines an effect of said anesthesia on said subject body by determining a relation between said measured response and one or more indications stored in said memory.
Example 39. A system according to any one of examples 36 to 38, wherein said control circuitry determines an effect of said anesthesia by activating said at least one pulse generator to generate and deliver two or more electric fields separated in time and/or in a stimulation location to said subject, by measuring a response of said subject body to the two or more electric fields, and by determining a relation between a first measured body response to a first electric field delivery, and a second body response to a second electric field delivery.
Example 40. A system according to example 39, wherein said control circuitry activates said pulse generator to generate and deliver two consecutive electric fields with an interval between the two consecutive electric field which is higher than 180 milliseconds.
Example 41. A system according to any one of examples 36 to 40, wherein an intensity of said generated electric field is in a range between 0.5 mA-40 mA and/or wherein a frequency of said generated electric field is in a range between 0.1 Hz-4000 Hz.
Example 42. A system according to any one of examples 36 to 41, comprising at least one user interface operationally connected to said control circuitry and configured to generate and deliver at least one human detectable indication to a user of the system and/or to an expert according to the determined anesthesia effect.
Example 43. A system according to example 42, wherein said at least one human detectable indication comprises an alert signal, and wherein said control circuitry signals said user interface to generate said alert signal if said determined anesthesia effect comprises a determined anesthesia depth that is not according to a planned anesthesia depth or indication thereof stored in said memory.
Example 44. A system according to example 42, wherein said at least one human detectable indication comprises an alert signal, and wherein said control circuitry signals said user interface to generate said alert signal if said determined anesthesia effect comprises a determined axial distribution of said anesthesia effect that is not according to a planned axial distribution or an indication thereof stored in said memory.
Example 45. A system according to example 42, wherein said control circuitry signals said user interface to generate said at least one human detectable indication with instructions to modify at least one parameter of said anesthesia according to said determined anesthesia effect.
Example 46. A system according to example 45, wherein said at least one parameter of said anesthesia comprises at least one of, administration site of one or more anesthetic compounds, dosage of said one or more anesthetic compounds, infusion rate of said one or more anesthetic compounds, ratio between two or more anesthetic compounds, and/or type of one or more anesthetic compounds.
Example 47. A system according to any one of examples 42 to 46, wherein said human detectable indication comprises a graphical representation of a distribution of said anesthesia effect and/or a graphical representation of a depth of said anesthesia in one or more body regions.
Example 48. A system according to any one of examples 42 to 47, wherein said control circuitry generates a pharmacodynamic profile of one or more anesthetic compounds used for said anesthesia in said subject, a trend of said anesthesia effect and/or a prediction of said anesthesia effect, based on said determined anesthesia effect and/or one or more subject or population-related indications stored in said memory.
Example 49. A system according to example 48, wherein said subject or population-related indications comprise one or more indications related to a clinical state of said subject or a population of individuals comprising at least one of, age, gender, BMI, medical history, drug regime, previously used stimulation parameter values, previously measured body response, previously determined anesthesia effect.
Example 50. A system according to any one of examples 48 or 49, wherein said control circuitry signals said user interface to generate a human detectable indication with instructions how to modify at least one parameter of said anesthesia and/or said stimulation according to at least one of, said determined anesthesia effect, said generated trend, said prediction, and/or said generated pharmacodynamic profile.
Example 51. A system according to any one of examples 48 or 49, wherein said control circuitry is configured to automatically modify at least one parameter of said anesthesia and/or at least one parameter of said stimulation according to at least one of, said determined anesthesia effect, said generated trend, said prediction, and/or said generated pharmacodynamic profile.
Example 52. A system according to any one of examples 36 to 51, comprising at least one actuator operationally connected to said control circuitry, wherein said actuator is configured to control an infusion rate of one or more anesthetic compounds into said subject body, and wherein said control circuitry is configured to automatically modify said at least one parameter of said anesthesia by controlling an activation of said at least one actuator.
Example 53. A system according to any one of examples 36 to 51, comprising at least one actuator operationally connected to said control circuitry, wherein said actuator is configured to control an infusion rate of one or more anesthetic compounds into said subject body, wherein said control circuitry automatically adjusts the activation of said actuator according to said determined anesthesia effect.
Example 54. A system according to example 53, wherein said control circuitry signals said actuator to stop or to reduce rate flow of one or more anesthetic compounds into said subject body if the determined anesthesia effect indicates distribution of said anesthesia effect towards unwanted body regions.
Example 55. A system according to any one of examples 34 to 56, comprising a communication circuitry operationally connected to said control circuitry and said memory; wherein said control circuitry signals said communication circuitry to transmit an indication to a remote device based on information stored in said memory.
Example 56. A system according to example 55 wherein said remote device comprises a remote computer, a remote display, a cloud storage, a remote server, a remote database.
Example 57. A system according to any one of examples 36 to 56, comprising an electrode patch having a surface configured to attach said electrode patch to a skin surface of said subject, wherein said electrode patch comprises said at least one stimulating electrode.
Example 58. A system according to example 57, wherein said at least one stimulating electrode comprises two or more stimulating arranged as an array in said electrode patch, and wherein each of said two or more stimulating electrodes in said array is separately electrically connected to said pulse generator.
Example 59. A system according to example 58, wherein a distance between two adjacent stimulating electrodes of said at least two stimulating electrodes is at least a distance between two adjacent dermatomes on a body of a subject or is at least a distance between two adjacent vertebra on a back of a subject.
Example 60. A system according to any one of examples 34 to 59 wherein said anesthesia comprises regional anesthesia or local anesthesia.
Example 61. A system according to any one of examples 34 to 60, wherein said memory stores one or more indications, and at least one data processing tool, and wherein said control circuitry is configured to process said one or more stored indications using said at least one data processing tool, wherein said data processing tool comprises at least one of, an algorithm, an algorithmic classifier, a software, and a lookup table.
Example 62. A system according to example 61, wherein said memory stores a database with information comprising at least one of, said one or more indications, results of said processing performed by said control circuitry, said measurements of a response of said subject body, and said determined anesthesia effect.
Example 63. A system according to example 62, wherein said one or more indications comprise indications regarding at least one of, previously measured responses of a subject body, previously used stimulation parameters, doses of anesthetic drugs, medical or clinical procedures where anesthesia delivery was used, personal details of one or more subjects receiving anesthesia in which an anesthesia effect was determined, clinical history and/or medical history of said one or more subjects, drug regime of said one or more subjects, and changes in an effect of anesthesia in one or more subjects during different medical or clinical procedures.
Example 64. A system according to any one of c examples 62 or 63, wherein said control circuitry is configured to determine said effect of anesthesia by determining a relation between said measured response of said body to said stimulation and said information in said database.
Example 65. A system according to any one of examples 62 to 64, wherein said control circuitry is configured to generate a trend or a prediction of an effect of said anesthesia on said subject by determining a relation between said determined effect of said anesthesia on said subject body and said information in said database.
Example 66. An electrode patch, comprising:
   a flexible body, wherein said flexible body is configured to conform to anatomical curvature of a human back, comprising:
   a skin contacting surface configured to be placed in contact with a skin surface of said subject back;
   two or more adjacent spaced-apart stimulating electrodes configured to deliver an electric field to said back tissue via said skin surface, wherein a distance between said two or more adjacent spaced-apart electrodes is predetermined according to a distance between two adjacent dermatomes of an adult human;
   at least one sensing electrode configured to sense a physiological response of said subject body, wherein a distance between said at least one sensing electrode and at least one stimulating electrode of said two or more adjacent stimulating electrodes is at least 2.5 times larger than a distance between said two or more stimulating electrodes.
Example 67. An electrode patch according to example 66, wherein a distance between said two or more stimulating electrodes is within a range between 2 cm-10 cm.
Example 68. An electrode patch according to any one of examples 66 or 67, wherein said two or more stimulating electrodes comprise at least 3 axially distributed stimulating electrodes arranged in an array.
Example 69. A method for determining a neural transmission related clinical state of a subject, comprising:
   stimulating a body of a subject at one or more stimulation sites;
   measuring a response of said subject to said stimulation, wherein said response passes through a nervous system of said subject;
   determining a clinical state and/or a stage of a clinical state of said subject based on said measured response, wherein said clinical state is related to neural transmission in said subject between two or more locations in a body of said subject.
Example 70. A method for determining an effect of local anesthesia in a subject, comprising:
   administering one or more anesthetic compounds at one or more administration sites, wherein said one or more anesthetic compounds are suitable for locally anesthetizing a target body region in said subject;
   stimulating said target body region of said subject at one or more stimulation sites within said target body region;
   measuring a response of said subject to said stimulation, wherein said response passes through a nervous system of said subject;
   determining an effect of said local anesthesia on said target body region based on results of said measuring.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as determining an anesthesia effect or anesthesia effect distribution, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart of a general process for determining of anesthesia effect, according to some exemplary embodiments of the invention;

FIGS. 2A-2D are schematic illustrations showing changes of anesthesia effect height in a subject body over time, according to some exemplary embodiments of the invention;

FIGS. 3A-3D are schematic illustrations showing changes in anesthesia effect depth, according to some exemplary embodiments of the invention;

FIGS. 4A-4C are graphs showing changes in anesthesia effect in an anesthesia target site over time with respect to medical procedure duration, according to some exemplary embodiments of the invention;

FIG. 4D is a graph showing changes in an optimal dosage of anesthetics over time, according to some exemplary embodiments of the invention;

FIG. 5A is a block diagram of a system for determining an effect of anesthesia, according to some exemplary embodiments of the invention;

FIG. 5B is a block diagram of a system communicating with remote devices and/or remote user interfaces, according to some exemplary embodiments of the invention;

FIG. 5C is a schematic illustration of a local user interface (LUI) of a system, according to some exemplary embodiments of the invention;

FIG. 5D is a schematic illustration of a Multi-Patients Remote User Interface (MRU) of a system, according to some exemplary embodiments of the invention;

FIG. 6 is a flow chart of a process performed by a user of a system for determining an effect of anesthesia, according to some exemplary embodiments of the invention;

FIG. 7 is a flow chart of a process performed by a system for determining an effect of anesthesia, according to some exemplary embodiments of the invention;

FIG. 8A is a schematic illustration showing an exemplary arrangement of stimulating electrodes and at least one sensing electrode, according to some exemplary embodiments of the invention;

FIG. 8B is a schematic illustration showing an additional exemplary arrangement of stimulating electrodes and at least one sensing electrode, according to some exemplary embodiments of the invention;

FIGS. 9A-9Z are schematic illustrations showing different arrangements of sensing and/or stimulating electrodes in an array or an electrode patch, for example a skin patch, according to some exemplary embodiments of the invention;

FIGS. 10A-10B are schematic illustrations showing locations of sensing electrodes measuring EEG signals on a head of a subject, according to some exemplary embodiments of the invention;

FIG. 10C is a schematic illustration showing locations of sensing electrodes measuring EEG signals on a head of a subject, according to some exemplary embodiments of the invention;

FIGS. 11A-11E are schematic illustrations showing location of stimulating electrodes and at least one sensing electrode, optionally for detection of neuropathy in a diabetic organ, for example a diabetic leg according to some exemplary embodiments of the invention; and

FIG. 12 is a schematic illustration showing detection of a local anesthesia effect, according to some exemplary embodiments of the invention.

### DESCRIPTION OF SPECIFIC EMBODIMENTS OF THE INVENTION

The present invention, in some embodiments thereof, relates to monitoring anesthesia and, more particularly, but not exclusively, to monitoring neuraxial anesthesia.

A broad aspect of some embodiments relates to measuring a response of a tissue to stimulation as an indication to an activity of sensory nerves delivering sensory information from the tissue. In some embodiments, a change in the measured signal following stimulation, for example a degradation in one or more signal parameters, indicates a reduction in sensory information transmission from the simulated tissue.

According to some embodiments, a degradation in the measured signal following stimulation indicates an effect of anesthesia, for example local or regional anesthesia, for example neuraxial anesthesia, on the stimulated tissue, and/or a pathological condition for example neuropathy of the stimulated tissue. In some embodiments, the degradation in the measured signal is determined by determining a relation between the measured signal and a previously measured reference or baseline signal, or indications thereof.

According to some embodiments, the response of tissue to stimulation is based on signals received from one or more electrodes attached to a subject body. Alternatively or additionally, the response of the tissue to stimulation is based on an input received from the subject, for example manual input, optionally received by a user interface.

An aspect of some embodiments relates to determining an effect of anesthesia on a subject, for example local anesthesia and/or regional anesthesia, for example neuroaxial anesthesia, by detecting a response of a body of the subject to stimulation. Alternatively or additionally, the effect of anesthesia on the subject is determined by detecting a change in sensory neural transmission. As used herein the term subject may refer to a human subject or to a non-human animal subject. As used herein, the term neuroaxial anesthesia refers to an example of regional anesthesia. In some embodiments, the body response to the stimulation is mediated by a nervous system of the subject body. In some embodiments, at least one parameter of anesthesia delivery is modified, optionally during a delivery of one or more anesthesia agents, according to the determined effect. In some embodiments, the at least one parameter comprises type and number of anesthesia agents, dosage of anesthesia and/or administration duration.

According to some embodiments, the system and/or method described herein is used for detecting and monitoring regional anesthesia and/or local anesthesia. In some embodiments, the system used for the detection and/or monitoring is configured to provide an indication, optionally online, whether a specific region is under the effect of the anesthesia or not. In some embodiments, providing an indication online comprises providing an indication in a time delay of less than 5 minutes, for example less than 1 minute, less than 30 seconds, less than 10 seconds, less than 2 seconds, less than 1 second, or any intermediate, shorter or longer time period from measuring a response of the body to the anesthesia.

According to some embodiments, the response of the body is detected by monitoring neural activity, for example by monitoring neural transmission, neural signal propagation and/or changes thereof. In some embodiments, the neural activity is monitored using at least one electrode, for example an electrode configured to measure an Event-Related Potential (ERP), for example electroencephalogram (EEG), or Somatosensory evoked potentials (SSEP), for example dermatomal SSEP. Alternatively or additionally, the body response is detected by monitoring muscle activity and/or changes thereof. In some embodiments, the muscle activity is monitored using at least one electrode, for example an Electromyography (EMG) electrode. In some embodiments, the response of the body to the stimulation optionally involves neurons of the nervous system of the subject, for example neurons in the spinal cord and/or neurons in the brain.

According to some embodiments, the effect of anesthesia is determined by determining a relation between a detected or a measured body response of a subject and one or more stored indication and/or one or more stimulation parameters. In some embodiments, the one or more stored indication comprises an indication of a body response previously measured in the same subject. Alternatively and additionally, the one or more stored indication comprises at least one indication of one or more body responses measured from different subjects that are optionally stored in a database. In some embodiments, the one or more stimulation parameters comprise at least one of, stimulation intensity, stimulation duration and/or stimulation frequency.

According to some embodiments, the effect of anesthesia is determined by determining a relation between an expected body response to the stimulation parameter with the actual response.

According to some embodiments, the body response is optionally detected at a site located at a distance from a stimulation site. In some embodiments, the response detection site is optionally located at a distance larger than 5 cm from a stimulation site, for example at a distance larger than 12 cm, larger than 15 cm, larger than 20 cm, larger than 30 cm, larger than 40 cm or any intermediate, smaller or larger distance from the stimulation site.

According to some embodiments, stimulation is provided to the body before and/or during the detection of the body response. In some embodiments, the stimulation is provided continuously or intermittently to the body. In some embodiments, the stimulation comprises at least one of delivery of an electric field to the body, delivery of an electric current to the body, delivery of tactile stimulation, vibration, thermal stimulation for example by delivery of thermal energy to the body, optical stimulation, pressure stimulation, puncture of the body or any combination thereof. In some embodiments, the stimulation comprises a sensory response evoking stimulation.

According to some embodiments, determining the effect of anesthesia comprises determining a distribution of the anesthesia effect in the subject body, for example based on levels of a signal or changes thereof measured by the at least one electrode, for example the EEG electrode and/or an EMG electrode, and/or any combination thereof. In some embodiments, the measured signal levels or changes thereof indicates whether one or more specific areas in the body are under an effect of the anesthesia, and/or what is the level of the anesthesia effect in the one or more specific body areas, for example what is the axial level along the spinal cord of the anesthesia effect. In some embodiments, the one or more specific body areas comprise one or more specific dermatomes. As used herein, a dermatome is an area of skin that is mainly supplied by a single spinal nerve. Each of these spinal nerves relay sensation from a particular region of the skin to the brain. In some embodiments, determining the distribution of the anesthesia effect comprises determining the distribution of the anesthesia effect over time and/or relative to a target location, for example an anatomical region in the body, and/or one or more dermatomes.

According to some embodiments, an effect of the anesthesia on at least one target body area, for example a target region in a body, is estimated based on the determined distribution of the anesthesia effect. In some embodiments, estimating the anesthesia effect on the target body area optionally comprises estimating whether the anesthesia effect on the target body area for example a current anesthesia effect, is a desired effect, for example a preplanned effect. According to some embodiments, the preplanned effect is optionally an anesthesia effect on the target body area that is required for a medical procedure, for example a surgery, and/or treatment of a clinical condition. In some embodiments, the preplanned effect is optionally an anesthesia effect with parameter values that are within a desired range of values, for example values that are higher than a minimal desired anesthesia effect, and lower than a maximal desired anesthesia effect. In some embodiments, a minimal desired anesthesia effect is a physiological effect on one or more of muscle system, nervous system and blood system of a subject, for example at the target body area, which is higher than a predetermined minimal value. In some embodiments, a maximal desired anesthesia effect is a physiological effect on one or more of muscle system, nervous system and blood system of a subject, for example at the target body area, which is lower than a predetermined maximal value.

According to some embodiments, the predetermined minimal and/or maximal values are optionally determined prior to the delivery of anesthesia and/or during the delivery of anesthesia. Alternatively and/or additionally, the predetermined minimal and/or maximal values are optionally determined according to changes in a treatment plan and/or according to changes in a surgical operation plan.

According to some embodiments, the minimal and/or maximal values are personalized for a specific subject, for example based on at least one clinical parameter of the patient. In some embodiments, the at least one clinical parameter comprises age, gender, weight, height, and/or BMI. Additionally or alternatively, the minimal and/or maximal values are determined, for example personalized for a specific subject, based on information collected from multiple subject, optionally stored in a database.

According to some embodiments, the effect of anesthesia on a subject body prior to and/or during childbirth is determined. In some embodiments, the anesthesia is administered prior to and/or during childbirth, for example in order to reduce pain sensation by a woman giving birth. In some embodiments, anesthesia, for example neuroaxial anesthesia is administered via ta least one site, for example an infusion site or an injection site in a body of the subject. In some embodiments, the at least one site is an insertion site of a needle used to deliver at least one anesthetic agent to the body. Optionally, the needle is inserted via the insertion site into an inner space between L2 and L4 vertebral bodies, for example to deliver or infuse the at least one anesthetic agent. In some embodiments, a target site for delivery of the stimulation is located in one or more dermatomes located between T10 to L5 dermatomes, for example T10, T11, T12, L1, L2, L3, L4, and L5 dermatomes. In some embodiments, when measuring somatosensory evoked potentials, for example dermatomal somatosensory evoked potentials (D-SSEP), at least one sensing electrode, for example an EEG electrode, is positioned on a head of the subject receiving the anesthesia, for example at locations located above, for example onto, cortical or sub-cortical brain regions. Alternatively or additionally, the at least one electrode is positioned, for example attached to a skin surface, onto a nape of the subject, at cervical locations, and to a skin surface of the back along the spinal cord. Alternatively or additionally, when measuring EMG, at least one electrode, for example an EMG electrode is positioned at facial muscle locations, onto one or more back muscles and/or neck. In some embodiments, the EMG electrode is attached to a skin surface above one or more facial muscles, one or more back muscles, and/or to a skin surface of the neck.

According to some embodiments, the effect of post and perioperative anesthesia on a subject body is determined. In some embodiments, the site of anesthesia administration is determined according to the type of medical procedure, for example surgery. In some embodiments, the anesthesia is administered to at least one inner space between L5 to T8 vertebral bodies. In some embodiments, a stimulation target site is determined according to the type of surgery and/or the injection site. Optionally, the target site for delivery of the stimulation is located in one or more dermatomes located between S5 to T2 dermatomes, for example S5, S4, S3, S2, S1, L5, L4, L3, L2, L1, T12, T11, T10, T9, T8, T7, T6, T5, T4, T3, and T2 dermatomes. In some embodiments, when measuring ERP, for example somatosensory evoked potentials (SSEP), for example dermatomal somatosensory evoked potentials (D-SSEP), at least one sensing electrode, for example an EEG electrode, is positioned on a head of the subject receiving the anesthesia, for example at locations located above cortical or sub-cortical brain regions. Alternatively or additionally, the at least one electrode is positioned, for example attached to a skin surface, onto a nape of the subject, above cervical locations, above a mastoid, on a head behind an car, for example on a head behind an car helix, and to a skin surface of the back along the spinal cord. Alternatively or additionally, when measuring EMG, at least one electrode, for example an EMG electrode is positioned at facial muscle locations, onto one or more back muscles and/or neck. In some embodiments, the EMG electrode is attached to a skin surface above one or more facial muscles, one or more back muscles, and/or to a skin surface of the neck.

According to some exemplary embodiments, the effect of anesthesia administered prior to, during and/or post a clinical intervention, for example a caesarean section procedure, child birth, and an orthopedic surgery on a subject body, is determined. In some embodiments, anesthesia, for example neuroaxial anesthesia is administered to at least one inner space between L2 and L4 vertebral bodies. In some embodiments, a target site for delivery of the stimulation is located in one or more dermatomes located between T4 to L5 dermatomes, for example T10, T11, T12, L1, L2, L3, L4, and L5 dermatomes. In some embodiments, when measuring ERP, for example somatosensory evoked potentials, for example dermatomal somatosensory evoked potentials (D-SSEP), at least one sensing electrode, for example an EEG electrode, is positioned on a head of the subject receiving the anesthesia, for example at locations located above cortical or sub-cortical brain regions. Alternatively or additionally, the at least one electrode is positioned, for example attached to a skin surface, onto a nape of the subject, at cervical locations, and to a skin surface of the back along the spinal cord. Alternatively or additionally, when measuring EMG, at least one electrode, for example an EMG electrode is positioned at facial muscle locations, onto one or more back muscles and/or neck. In some embodiments, the EMG electrode is attached to a skin surface above one or more facial muscles, one or more back muscles, and/or to a skin surface of the neck.

According to some embodiments, the effect of anesthesia on a subject body when treating chronic pain is determined. In some embodiments, the anesthesia is administered to at least one inner space between L2 and L3 vertebral bodies. In some embodiments, the target site for delivery of the stimulation is determined according to the location of the pain and/or the injection site. Optionally, the target site for the delivery of stimulation is located in one or more dermatomes between S5 to T2 dermatomes, for example S5, S4, S3, S2, S1, L5, L4, L3, L2, L1 and T12 dermatomes. In some embodiments, when measuring ERP, for example somatosensory evoked potentials, for example dermatomal somatosensory evoked potentials (D-SSEP), at least one sensing electrode, for example an EEG electrode, is positioned on a head of the subject receiving the anesthesia, for example at locations located above cortical or sub-cortical brain regions. Alternatively or additionally, the at least one electrode is positioned, for example attached to a skin surface, onto a nape of the subject, at cervical locations, and to a skin surface of the back along the spinal cord. Alternatively or additionally, when measuring EMG, at least one electrode, for example an EMG electrode is positioned at facial muscle locations, onto one or more back muscles and/or neck. In some embodiments, the EMG electrode is attached to a skin surface above one or more facial muscles, one or more back muscles, and/or to a skin surface of the neck.

According to some embodiments, as described above, a stimulation is delivered to a tissue, for example in order to determine an anesthesia effect or a pathological state of the stimulated tissue. In some embodiments, the stimulation is delivered with parameter values, for example intensity, frequency and/or duration, which are sufficient to induce transmission of sensory signals from the stimulated tissue, for example neural transmission of sensory signals from the stimulated tissue. In some embodiments, at least one signal is recorded following the stimulation from a site which is different from the stimulation site, for example to determine an ability of sensory nerves in the tissue to generate and transmit the sensory signals from the stimulated tissue.

According to some embodiments, an anesthesia effect is determined according to a correlation between a response of the body to stimulation and one or more stimulation parameters.

According to some embodiments, the delivered stimulation comprises delivery of an electric field to at least one stimulation site with an intensity in a range of 0-40 mA, for example 0.5-40 mA, 0-10 mA, 5-20 mA, 15-40 mA or any intermediate, smaller or larger range of values. Optionally, stimulation is delivered with an intensity in a range of 2 to 9 mA, for example 2 to 5 mA, 5-8 mA or any intermediate, smaller or larger range of values. In some embodiments, when stimulating a non-anesthetized tissue, the electric field is delivered with a stimulation intensity in a range between 1-4 mA, which is sufficient to generate and deliver sensory neural signals from the tissue, for example to the brain. In some embodiments, when stimulating an anesthetized tissue, the electric field is delivered with an intensity value in a range between 5-8 mA, in order to induce the generation of sensory neural signals from the brain.

According to some exemplary embodiments, when measuring a signal, for example D-SSEP signals in cortical and/or in sub-cortical locations in response to stimulation, measurement is performed in up to 300 milliseconds (ms) after the end of the stimulation, for example measurement is performed in up to 200 milliseconds (ms) after the end of the stimulation, for example measurement is performed in up to 100 milliseconds (ms) after the end of the stimulation, for example measurement is performed in up to 50 milliseconds (ms) after the end of the stimulation, for example measurement is performed in up to 20 milliseconds (ms) after the end of the stimulation, for example measurement is performed in up to 15 milliseconds (ms) after the end of the stimulation or any intermediate, smaller or larger value. Alternatively, measurement is performed up to 300 ms after the initiation of the stimulation.

According to some embodiments, the anesthesia effect, for example regional anesthesia effect, on one or more target regions in the body. In some embodiments, the one or more target regions are target regions associated with one or more stimulation sites. Alternatively or additionally, the target regions are target regions located at a distance from the one or more stimulation sites or between two adjacent stimulation sites.

Without being bound by any theory or mechanism of action, an anesthetic drug affecting a tissue elevates a sensory threshold of the tissue, therefore a stronger stimulation is needed in order to generate and deliver sensory neural signals by the tissue. In some embodiments, an anesthesia effect on a tissue, for example an anesthesia depth and/or an anesthesia height is determined based on a change in stimulation needed to generate and deliver sensory neural signals from the tissue. Alternatively or additionally, the anesthesia effect on the tissue is determined by detecting a degradation in a measured signal, for example a reduction in a signal amplitude. Alternatively or additionally, the anesthesia effect on the tissue is determined by detecting changes in one or more parameters of the sense signal, for example, changes in signal shape and/or changes in signal duration and/or changes in relations between signal's peaks, and/or changes in signal threshold crossings, in a response to a stimulation, for example a stimulation delivered with similar stimulation parameters before and after anesthetics delivery.

According to some embodiments, when measuring neural signals, for example D-SSEP signals in response to stimulation, the delivered electric field stimulation signal is modulated such that a signal polarity is biphasic, inverse, normal and/or any combination thereof. In some embodiments, the signal amplitude, for example intensity is in a range of 0-40 mA, and the signal frequency is in a range of up to 4000 Hz.

According to some exemplary embodiments, an anesthesia effect for example anesthesia depth is determined by detecting transmission of sensory neural signals by specific neural fibers. In some embodiments, each neural fiber type delivers different type of sensory information, for example temperature sensation, sharp pain sensation (prick), blunt pain sensation, tactile sensation, motoric sensation, and Proprioception. In some embodiments, the different types of neuron fibers respond differently to different types of electrical stimulations. In some embodiments, different frequency ranges of the stimulating electric field induce generation and transmission of sensory neural signals through specific neural fibers. In some embodiments, a stimulating electric field with a frequency in a range of about 1800 Hz to about 4000 Hz, for example 1900 Hz to 2100 Hz, induce neural transmission of sensory signals via A-beta fibers. In some embodiments, a stimulating electric field with a frequency in a range of about 220 Hz to about 280 Hz, for example 240 Hz to 260 Hz, induce neural transmission of sensory signals via A-delta fibers. In some embodiments, a stimulating electric field with a frequency in a range of about 3 Hz to about 7 Hz, for example 4 Hz to 6 Hz, induce neural transmission of sensory signals via C fibers.

Optionally, a shape of a measured EEG and/or EMG signal indicates a depth of anesthesia.

According to some embodiments, determining an effect of anesthesia on a tissue comprises predicting of anesthesia effect on other tissues and over time. In some embodiments, the signals received from one or more sensing electrodes, following stimulation delivery to tissue, are analyzed for example to identify patterns in the received signals that can be correlated with the delivery of stimulation to the tissue. In some embodiments, the identified patterns are compared to previously identified patterns, optionally stored in a database, for example an external database, and/or in a memory of the system, or indications thereof. In some embodiments, one or more algorithms, for example machine learning algorithms are used to identify patterns or indications thereof which correlate with a predicted anesthesia effect on the stimulated tissue or on a different tissue of the body, are used for feature analysis of acquired detected signals, are used for prediction of anesthesia trend, and/or for a closed-loop of the system means of operation.

According to some embodiments, determining an effect of anesthesia during the delivery of anesthesia, for example by infusion of anesthetic agents, for example drugs, into the body, allows, for example to personalize the anesthesia delivery by controlling in real time the infusion of the anesthetic agents into the subject body. In some embodiments, the system for determining an anesthesia effect, for example a control unit of the system, is coupled to an actuator, for example a pump or a syringe that actively infuses the anesthetic agents into the subject body. In some embodiments, a control circuitry of the system is configured to control the activation of the actuator based on the determined anesthesia effect, optionally in a closed-loop process, for example to allow on one hand effective and desired anesthesia effect and on the other hand to minimize side effects.

Potential advantages of controlling on-line an infusion process of anesthetic agents based on a determined anesthesia effect, may include personalized dosing of anesthetic agents, improvement of a childbirth experience by reducing side effects, and/or automatically identification at high level of accuracy whether a sensation has been sensed by the anesthetized subject or not.

Additional potential advantage of an automatic system for determining an anesthesia effect, thereby monitoring anesthesia effect in one or more patients, may include a closed-loop process without involvement of an expert, personalized treatment, remote monitoring of the anesthesia effect and/or anesthesia process progression, display anesthesia status during childbirth or any other medical or clinical procedure, and/or multi-patient monitoring.

An aspect of some embodiments relates to collecting and processing data related to a response of subjects to anesthesia protocols. In some embodiments, the data comprises electrode measurements or indications thereof of a body response to anesthesia protocols provided to each subject. In some embodiments, the data is collected into a database. Optionally processing of the collected data is performed in the database, by one or more algorithms, for example machine learning algorithms, artificial intelligence models, statistical evaluation, and regression models etc. In some embodiments, the collected and/or processed data is used to update existing anesthesia protocols or parameters thereof and/or to generate new anesthesia protocols for specific therapeutic applications. Alternatively or additionally, the collected and/or processed data is used to personalize an anesthesia protocol for a specific subject, optionally for a specific therapeutic application.

According to some embodiments, the collected data includes personal and/or clinical data from each subject receiving anesthesia, for example age, gender, height, weight, BMI, percentage of fat tissue in the body, medical history, list of drugs administered to the subject, and/information regarding a surgical procedure performed on the subject. Optionally, the collected data includes at least one physiological parameter measured before, during, and/or after anesthesia administration, for example, heart rate, and/or blood pressure. Alternatively or additionally, the collected data comprises a dosage, amount, type and/or combination of anesthetic compounds used in the anesthesia procedure and/or information with regard to neural transmission in the subject.

An aspect of some embodiments relates to determining of a clinical state and/or a stage of a clinical state, by measuring a body response to a stimulation. In some embodiments, the response of the subject passes or is mediated by the nervous system of the subject. In some embodiments, measuring of a subject response comprises measuring at least one ERP, for example measuring at least one EMG signal and/or at least one SSEP signal following stimulation delivery. In some embodiments, the clinical state and/or a stage of the clinical state is determined based on a determined relation between the measured subject response and one or more indications stored in a memory. In some embodiments, the stored one or more indications comprise indications of one or more measured responses from the same subject, and/or or indications of one or more measured responses from different subjects.

According to some embodiments, the clinical state and/or a stage of the clinical state is determined based on a relation between a measured response of an anesthetized body tissue compared to a measured response an un-anesthetized body tissue. Alternatively or additionally, the clinical state and/or a stage of the clinical state is determined based on a relation between measurements of a body response at different time points. Alternatively or additionally, the clinical state and/or a stage of the clinical state is determined based on a relation between a measured subject response of a body tissue and a stored pharmacodynamics profile.

Optionally, the response of the subject is measured after each stimulation of two or more consecutive stimulations, for example stimulations at different stimulation sites. In some embodiments, the clinical state and/or a stage of the clinical state is determined based on a change between a response measured after a first stimulation and a response measured after a second stimulation. In some embodiments, the response of the subject is measured by measuring ERP, for example EMG and/or SSEP.

According to some embodiments, the clinical state comprises peripheral neuropathy, for example peripheral neuropathy in a diabetic organ, for example a diabetic leg. In some embodiments, at least one stimulation is delivered at one or more stimulation locations along a leg. In some embodiments, a relation is determined between at least one signal measured following the at least one stimulation and at least one indication stored in a memory. In some embodiments, a reduction or blockage in neural transmission from the one or more stimulation locations is determined based on the determined relation. Alternatively, two or more stimulations are delivered, each at a different stimulation location along a leg of the subject. In some embodiments, a reduction in one or more parameters of a signal measured following stimulation at a first stimulation site, compared to at least one different signal measured following stimulation at a second stimulation site on a leg, indicates a reduction or blockage in neural transmission in the between the first stimulation site and a measurement site where the signal was measured.

According to some embodiments, the system described herein monitors an anesthesia effect on a subject by delivering a stimulation to a subject while the subject is anesthetized, and detecting a response of the subject to the stimulation. In some embodiments, the system determines the anesthesia effect continuously, while the subject receives at least one anesthetic agent, for example drug. In some embodiments, the system provides at least 5 indications within a time period of at least 5 minutes, regarding the anesthesia effect. In some embodiments, the subject receiving local anesthesia and/or regional anesthesia. In some embodiments, the stimulation is delivered by at least one stimulating electrode or by a plurality of stimulating electrodes, optionally arranged in an array. In some embodiments, the response of the subject to the stimulation is detected by at least one electrode contacting the body of the subject and/or by receiving input from the subject using for example a user interface of the system.

According to some embodiments, the system monitors the anesthesia effect using one or more algorithms, for example algorithmic classifiers, stored in a memory associated with the system, for example a memory of a remote device or a memory of the system. In some embodiments, the system uses the one or more algorithms to determine an anesthesia effect of the subject at a specific time, to generate a prediction regarding the anesthesia effect on the subject in the future, generate a trend of the anesthesia effect over time, optionally a predicted trend, and/or generate a pharmacodynamic profile of one or more anesthetic agents, for example drugs, for the specific patient.

According to some embodiments, the system uses the one or more data processing tools, for example algorithms, machine learning algorithms, algorithmic classifiers, statistical tools, artificial intelligence tools, and/or regression models, for monitoring of the anesthesia effect over time, optionally in a closed loop process.

According to some embodiments, the closed loop process includes at least one of, modifying at least one parameter of the stimulation, modifying at least one parameter of the anesthetic agents administration, modifying at least one parameter of the anesthesia effect monitoring, delivering of human detectable indications regarding the anesthesia effect in a subject and optionally a predicted anesthesia effect in the subject, delivering alerts if the anesthesia effect monitoring indicates or predicts an effect which is lower than a target, for example a desired, anesthesia effect.

According to some embodiments, the closed loop process comprises replacing at least one processing tool with a different processing tool based on the signals received from the at least one sensing electrode and/or information stored in the memory. In some embodiments, the information stored in the memory comprises at least one of, clinical data of the subject, medical history of the subject, information regarding the medical procedure, information regarding childbirth, previously received data from at least one sensing electrode, previously generated predictions and/or trends, parameter values of a planned anesthesia effect optionally per subject and/or per clinical procedure for example medical procedure or childbirth, age of the subject, sex of the subject, drugs received by the subject, information regarding previous anesthesia monitoring procedures or regarding previous anesthesia procedures in the subject. In some embodiments, the information stored in the memory is subject specific and/or population specific,

According to some embodiments, the system automatically modifies, for example in a closed loop process, at least one parameter of the monitoring process and/or at least one parameter of the anesthesia delivered to the subject and/or at least one stimulation parameter. In some embodiments, the system automatically modifies the monitoring process parameter and/or the anesthesia parameter and/or the stimulation parameter based on a current determined anesthesia effect, and/or based on an anesthesia effect trend over time and/or based on a prediction of the anesthesia effect.

In some embodiments, the monitoring process parameter comprises at least one of, type of a sensing electrode, number of sensing electrodes, position of sensing electrodes, type of algorithm used for processing signals received from the sensing electrode, type of algorithm used for monitoring anesthesia effect, frequency of generating and delivering or updating indications regarding anesthesia effect in a patient, and type of human detectable indication.

In some embodiments, the stimulation parameter comprises at least one of, stimulation intensity, stimulation duration, stimulation frequency, number of stimulation electrodes, and position of stimulation electrodes.

In some embodiments, the anesthesia parameter comprises at least one of, location for administering at least one anesthetic agent, type of anesthetic agent, number of anesthetic agents, dose of at least one anesthetic agent, duration of the administering of the anesthetic agent.

According to some embodiments, the system is used to generate a database which includes information collected from a plurality of subjects, for example patients that used the anesthesia effect monitoring system. In some embodiments, the database is stored in a memory associated with the system, for example a memory of the system or a memory of a remote device. In some embodiments, the system, for example a control circuitry of the system uses the information stored in the database in order to, determine an effect of the anesthesia on a specific subject, and/or generate a trend or a prediction of an anesthesia effect on the specific subject.

The database includes information that is processed, for example to optimize at least one algorithm that is used for anesthesia effect monitoring, classifying and/or anesthesia effect prediction. In some embodiments, the database is generated based on input received from the anesthesia effect monitoring system, input received from an additional system and/or input received from a subject, for example an expert, a physician, a nurse, a caregiver or a patient.

According to some embodiments, the database includes information, for example subject-specific information regarding at least one of, administration method of at least one anesthetic agent, type of anesthetic agent, dosage of the anesthetic agent, physiological parameter values of a subject receiving the anesthetic drug, for example a woman undergoing childbirth, BMI scores of the subject, age of subject, whether this is a first childbirth of the subject, comorbidity of the subject with the anesthetic agent, background diseases of the subject, neuronal injury of the subject, neuronal injury in a planned site for the anesthetic administration, whether this is a first time of the subject in receiving regional anesthesia, for example epidural anesthesia, any known reported side effects of the anesthetic agent in the subject, any general side effects, medical history, background diseases, age, alcohol consumption, drug consumption, caffeine consumption, food supplements consumption, drugs consumption, and type and/or dose of pain killers received by the subject prior to receiving regional anesthesia.

According to some embodiments, the information in the database is processed using one or more algorithms and/or statistical tools, in order to classify and/or categorize the information per specific populations of subjects.

An aspect of some embodiments relates to an electrode patch configured to be attached to a skin surface of a body tissue, having two or more spaced-apart stimulating electrodes configured to deliver an electric field to the body tissue. In some embodiments, a distance between the two or more spaced-apart stimulating electrodes is predetermined according to a distance between two adjacent dermatomes of a human subject, for example an adult human subject or according to a distance between two adjacent dermatomes of a child. In some embodiments, the electrode patch comprises at least one sensing electrode, configured to record a signal from the subject body, for example a response of the subject body to the delivered electric field.

According to some exemplary embodiments, a distance between the two or more stimulating electrodes is in a range between 2 cm to 15 cm, for example 2 cm to 5 cm, 3 cm to 7 cm, 1.5 cm to 4 cm or any intermediate, shorter or longer distance. In some embodiments, a shortest distance between the at least one sensing electrode to at least one stimulating electrode of the two or more stimulating electrodes is at least 2 times larger, for example at least 2.5 times, at least 3 times, at least 3.5 times, at least 5 times or any intermediate, smaller or larger value, than a shortest distance between the two or more stimulating electrodes. In some embodiments, the shortest distance between the at least one sensing electrode and at least one stimulating electrode of the two or more stimulating electrodes is in a range between 10 cm to 150 cm, for example in a range between 10 cm to 60 cm, in a range between 20 cm to 50 cm, in a range between 25 cm to 70 cm or any intermediate, sorter or larger distance.

According to some embodiments, the two or more stimulating electrodes comprise 3 or more stimulating electrodes, for example 4, 5, 6, 7, 8, 9, 10 or any larger number of stimulating electrodes, optionally arranged as an array. In some embodiments, the 3 or more stimulating electrodes are axially distributed and spaced-apart from each other in the electrode patch, for example in a patch body. In some embodiments, the patch body is flexible, to conform to an anatomy of a back of a subject. In some embodiments, the patch, for example the patch body has a skin contacting surface configured to be attached to a skin surface of the subject body, for example to a skin surface of the back of the subject. Optionally, the patch is elastic. Optionally, the patch comprises at least one stimulating portion comprises the two or more stimulating electrodes and at least one sensing portion comprising the at least one sensing electrode.

According to some embodiments, the at least one stimulating portion comprises two or more spaced apart stimulating portions, having an opening therebetween. In some embodiments, the opening, for example a void or a window, is larger than an injection site of anesthetic agents in the subject body. In some embodiments, stimulating electrodes of a first stimulating portion are aligned relatively to stimulating electrodes of a second stimulating portion.

According to some embodiments, the anesthesia effect monitoring and/or the anesthesia effect determined by the methods and the systems described herein is performed in a subject that is awake, for example in a subject that is not under general anesthesia.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Process for Determining of Anesthesia Effect

According to some exemplary embodiments, an effect of anesthesia, for example regional anesthesia, local anesthesia and/or neuroaxial anesthesia is determined using a system. In some embodiments, the anesthesia effect is determined in a subject that is sedated or anesthetized. The subject is optionally awake. In some embodiments, the anesthesia effect is determined by receiving information from a body of the subject, for example from at least one electrode connected to the body of the subject, and optionally processing and/or analyzing the received information. The system is configured to provide an indication, for example a human detectable indication, whether a specific region of the body is anesthetized or not. The system is configured to classify an anesthesia effect by determining a relation between a stimulation delivered to a tissue and a response measured from the tissue, following the stimulation. The stimulation is delivered repeatedly within a time interval determined by at least one algorithm or at least one protocol stored in a memory of the system. The system determines which stimulating electrode or a plurality of electrodes to use every stimulation cycle.

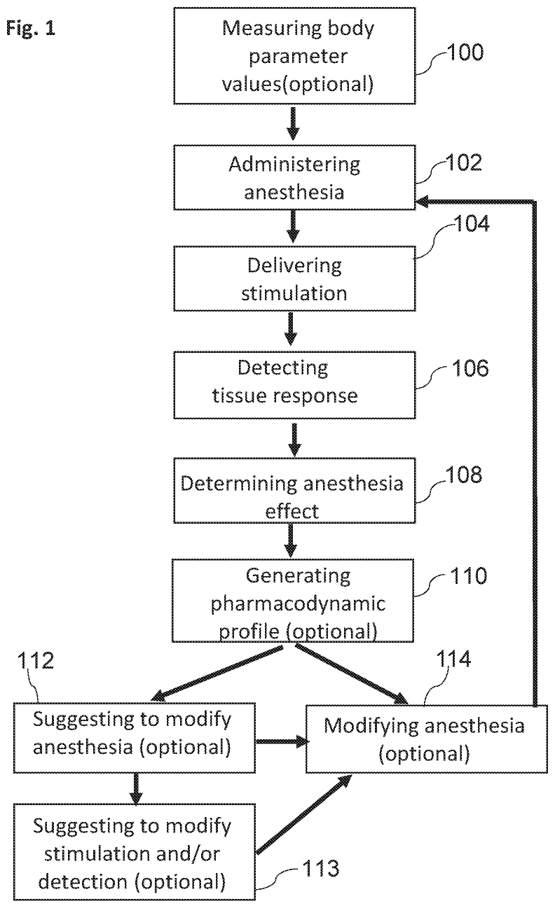

Reference is now made to FIG. 1 depicting a general process for determining of anesthesia effect on a body of a subject, according to some exemplary embodiments of the invention. In some embodiments, the determining of anesthesia effect is performed in an operating room, in a treatment room or at the subject home.

According to some exemplary embodiments, values of one or more body parameters are optionally measured at block 100. In some embodiments, the one or more body parameter values are measured by at least one electrode attached to a surface of the subject skin or inserted into a body tissue. In some embodiments, the at least one electrode is positioned at a target location on the body. In some embodiments, the at least one electrode comprises an EEG and/or an EMG electrode. In some embodiments, the one or more body parameter values measured at block 100 are used as baseline or reference values for future measurements by the at least one electrode and/or at least one different electrode.

According to some exemplary embodiments, the measurement values measured at block 100 are baseline values measured from a tissue prior to administering anesthesia, for example to indicate measured electrical noise values of the tissue prior to anesthesia administration and/or measured electrical noise values due to electromagnetic waves in the vicinity of the subject. Alternatively or optionally, the measurement values are from tissue which is not anesthetized. According to some exemplary embodiments, anesthesia, for example neuroaxial anesthesia and/or regional anesthesia, is administered to a body of the subject, at block 102. In some embodiments, the anesthesia is administered before, during and/or after a medical procedure, for example a surgical procedure. Alternatively or additionally, the anesthesia is administered to treat a clinical condition for example to treat childbirth pain, to treat postoperative pain, to treat operative pain for example pain during caesarian section, orthopedic surgery or other surgical procedures, to treat chronic pain. Alternatively, the anesthesia comprises local and/or regional anesthesia delivered to anesthetized a specific anatomical region, for example a specific limb, or a section of a limb.

According to some exemplary embodiments, anesthesia is administered at block 102 by optionally administering, for example infusing and/or injecting, one or more anesthetizing agents into the subject body through one or more body entry sites, for example one or more infusion or injection sites. In some embodiments, the anesthesia is administered into an epidural space between two vertebral bodies of a spinal cord. In some embodiments, the one or more anesthetizing agents comprise local anesthetics, for example Bupivacaine and Lidocaine, Opioids for example Morphine, and Fentanyl, Clonidine, Epinephrine or any combination thereof. In some embodiments, the one or more anesthetizing agents are administered according to a at least one administering parameter comprising dose of the one or more agents, ratio between two or more anesthetizing agents, administering duration, and/or type of the one or more agents.

According to some exemplary embodiments, the at least one anesthesia administration parameter is predetermined prior to and according to a planned surgical procedure, for example according to a Cesarean section, or according to an abdomen surgery, orthopedic surgery or other surgical procedures. Alternatively, or additionally, the at least one anesthesia administration parameter is predetermined prior to cervical effacement and/or opening of the cervix and/or prior to movement of a baby through a birth canal. In some embodiments, the at least one anesthesia administration parameter is predetermined according to a clinical and/or a physiological state of a subject, for example according to height, weight, BMI, age, gender, medical history and/or medications taken by the subject.

According to some exemplary embodiments, the anesthesia is administered into the body of the subject continuously or intermittently. In some embodiments, the anesthesia is administered in a fixed dosage or in different dosages that vary throughout the anesthesia administration period.

According to some exemplary embodiments, stimulation is delivered to the body of the subject, at block 104. In some embodiments, the stimulation is delivered with parameter values, for example intensity, duration and/or frequency that are sufficient to evoke a response, for example a sensory response of the body. In some embodiments, the stimulation comprises an electric stimulation, a thermal stimulation, a pressure stimulation, a tactile stimulation, a visual stimulation, an audio stimulation or any combination thereof.

According to some exemplary embodiments, the stimulation is delivered continuously and/or intermittently. In some embodiments, the stimulation is delivered as repetitive pulses or a repetitive sequence of pulses. Optionally, the delivered stimulation is modulated, for example the number of stimulation pulses is modified, at least one stimulation parameter is modified, time between stimulation pulses is modified, optionally according to measurements from the body of the subject and/or according to indication stored in a memory of the system.

According to some exemplary embodiments, the stimulation, for example an electric stimulation is delivered to one or more target sites, for example one or more stimulation sites, of the body. In some embodiments, the one or more target sites are located at one or more dermatomes. Optionally, the electric stimulation s delivered to a skin surface of the one or more dermatomes. In some embodiments, the one or more target sites comprise a plurality of target sites axially distributed along a dermatomes axis. Optionally, each target site is located at a different dermatome. Alternatively, two or more target sites are located at the same dermatome.

According to some exemplary embodiments, the electric stimulation is delivered at block 104 to the one or more target sites with at least one parameter selected to evoke an ERP, for example a sensory response, for example a somatosensory response. In some embodiments, the at least one parameter of the electric stimulation comprises intensity, frequency, and/or duration of the electric stimulation.

According to some exemplary embodiments, the electric stimulation is delivered to the at least one target site with an intensity in a range between 0 milliampere (mA) to 40 mA, for example in a range between 0-10 mA, 0-5 mA, 5-20 mA, 10-30 mA, 20-40 mA, or any intermediate, smaller or larger range of values. In some embodiments, the electric stimulation is delivered in a range between 2-10 mA, for example 2-8 mA, 5-10 mA, 2-5 mA or any intermediate, smaller or larger range of values, which is sufficient to induce a somatosensory response in a subject.

According to some exemplary embodiments, the electric stimulation is delivered with a frequency in a range of 1-4000 Hz, for example 1-100 Hz, 50-500 Hz, 100-1000 Hz, 100-2000 Hz, 100-3000 Hz, 500-1000 Hz, 500-2000 Hz, 500-3000 Hz, 1000-2000 Hz, 1000-3000 Hz. 2000-3000 Hz. 3000-4000 Hz or any intermediate, smaller or larger range of values. In some embodiments, each electric stimulation is delivered for a duration in a range of 0.1-10 milliseconds (ms), for example 1-5 ms, 2-6 ms, 5-10 ms or any intermediate, smaller or larger range of values.

According to some exemplary embodiments, each stimulation pulse is delivered with a duration in a range of 0-1000 microseconds, for example 0-500 microseconds, 300-600 microseconds, 400-1000 microseconds, or any intermediate, smaller or larger value.

According to some exemplary embodiments, an interval between two consecutive stimulation pulses is higher than a synaptic fatigue duration, for example higher than 180 milliseconds.

According to some exemplary embodiments, a response of a body tissue is detected at block 106. In some embodiments, the body tissue response is detected by measurement of the tissue response at block 106. In some embodiments, the tissue response is measured using one or more sensors, for example one or more electrodes configured to sense at least one parameter of the tissue, for example tissue movement, tissue temperature, electrical conductivity of the tissue, electrical signals generated by the tissue, electrical signals transmitted by the tissue, electrical signals received by the tissue, and/or concentration of chemical compounds in the tissue.

Alternatively or additionally, the response of the body tissue is detected based on input received from the subject, optionally manually using a user interface. Optionally, the input is received from the subject and is entered to the system by a different subject, for example by a caregiver, an expert or a nurse.

According to some exemplary embodiments, the tissue response is measured by at least one sensor configured to measure Electromyography (EMG). In some embodiments, the at least one EMG sensor measures muscle response or electrical activity in response to a nerve's stimulation of the muscle. Alternatively or additionally, the tissue response is measured by at least one sensor configured to measure an ERP, for example somatosensory evoked potentials (SSEPs) or dermatomal SSEPs (D-SSEP). In some embodiments, measuring an ERP, for example, measuring D-SSEP allows, for example to examine ERPs, for example SSEPs from individual dermatomes, which optionally correspond with specific spinal segments.

According to some exemplary embodiments, the tissue response is detected, for example measured, following and/or during the stimulation delivered at block 104.

According to some exemplary embodiments, the tissue response is detected at block 106 by determining a relation between measurements performed at block 106 and reference measurements or baseline measurements optionally performed at block 100. Alternatively or additionally, the tissue response is detected by determining a relation measurements performed at block 106 or indications thereof, and one or more indications stored in a memory, for example a memory of a remote device or a memory of an anesthesia monitoring system. Optionally, the parameters measured at block 106 and in block 100 are the same parameters. Optionally, the parameter values measured at block 106 are compared to the parameter values measured at block 100 in order to determine the relation between the two measurements. In some embodiments, the reference measurements comprise a reference scale or reference values or indications thereof, which are optionally based on information collected from a plurality of subjects. In some embodiments, the relation between measurements performed at block 106 and stored indications and/or additional measurements is determined using one or more algorithms, for example machine learning algorithms.

According to some exemplary embodiments, at least one parameter of the anesthesia effect, for example anesthesia effect distribution, is determined at block 108. In some embodiments, the anesthesia effect is determined based on the detected tissue response, for example measured tissue response. Alternatively or additionally, the at least one parameter of the anesthesia effect is determined based on the determined relation between the measurements performed at block 106 and the reference values or baseline values measured at block 100.

According to some exemplary embodiments, measuring of the tissue response allows for example to determine the effect of anesthesia on a target tissue. For example to determine if the effect of anesthesia on the target tissue is according to a treatment plan, for example a personalized treatment plan.

According to some exemplary embodiments, determining if the effect of anesthesia on the target tissue is according to a treatment plan comprises determining if the effect of anesthesia is according to a predetermined treatment plan, determining if the effect of anesthesia is a desired effect on a selected target tissue, for example at a specific time point.

According to some exemplary embodiments, the anesthesia effect is determined in order to determine the anesthesia effect on the selected target tissue prior to and/or during a treatment process and/or a surgical procedure. In some embodiments, the anesthesia effect on a target tissue is determined by measuring response of a tissue in at least one measurement site, to stimulation transmitted in at least one stimulation site.

According to some exemplary embodiments, the anesthesia effect is determined, for example, in order to determine the effect of anesthesia on one or more dermatomes, for example to determine a depth of anesthesia in the one or more dermatomes.

According to some exemplary embodiments, a pharmacodynamic profile is optionally generated at block 110. In some embodiments, the pharmacodynamics profile is generated based on the effect of anesthesia determined at block 108 and at least one anesthesia parameter, for example anesthesia dosage, anesthesia administration regime, anesthesia administration location, type of bioactive compounds used for anesthesia, combination of the bioactive compounds, and/or side effects of the anesthesia administration. In some embodiments, the generated pharmacodynamics profile is personalized for a specific subject. Optionally, the pharmacodynamics profile is generated in a local and/or in a remote device, for example a remote storage device, or a cloud. In some embodiments, the pharmacodynamic profile includes information regarding one or more dermatomes affected by the anesthesia, and/or the depth of anesthesia at the one or more dermatomes.

According to some exemplary embodiments, a suggestion to modify at least one parameter of the anesthesia is optionally generated at block 112. In some embodiments, the suggestion is generated by an anesthesia monitoring device. Optionally, the suggestion to modify the at least one parameter of the anesthesia is transmitted to a different device, for example a device located in a treatment room, a device located at an operating room, or to a device used to monitor a clinical state of one or more patients.

According to some exemplary embodiments, the suggestion to modify the at least one anesthesia parameter is generated based on the determined distribution of the anesthesia effect and/or based on the anesthesia effect on a target tissue. Alternatively or additionally, a suggestion to modify at least one parameter of the anesthesia is generated based on the tissue response detected and/or measured at block 106. Alternatively or additionally, a suggestion to modify at least one parameter of the anesthesia is generated based on the subject response, for example based on input received from the subject. Alternatively or additionally, a suggestion to modify the at least one anesthesia parameter is generated based on the pharmacodynamics profile generated at block 110.

According to some exemplary embodiments, the suggestion to modify the at least one anesthesia parameter comprises suggestion to stop anesthesia administration to the body, modify anesthesia dosage, modify a ratio between two or more bioactive compounds, for example anesthetic drugs, in the anesthesia and/or add or remove at least one bioactive compound from the anesthesia.

According to some exemplary embodiments, a suggestion to modify at least one parameter of the stimulation and/or of the detection of the tissue response is optionally generated at block 112. In some embodiments, the suggestion is generated by an anesthesia monitoring device. Optionally, the suggestion to modify at least one parameter of the stimulation and/or of the detection of the tissue response is transmitted to a different device, for example a device located in a treatment room, a device located at an operating room, or to a device used to monitor a clinical state of one or more patients.

According to some exemplary embodiments, the suggestion to modify the at least one parameter of the stimulation and/or of the detection of the tissue response is generated based on the determined distribution of the anesthesia effect and/or based on the anesthesia effect on a target tissue. Alternatively or additionally, a suggestion to modify the at least one parameter of the stimulation and/or of the detection of the tissue response is generated based on the tissue response detected and/or measured at block 106. Alternatively or additionally, the suggestion to modify the at least one parameter of the stimulation and/or of the detection of the tissue response is generated based on the subject response, for example based on input received from the subject. Alternatively or additionally, the suggestion to modify the at least one parameter of the stimulation and/or of the detection of the tissue response is generated based on the pharmacodynamics profile generated at block 110.

According to some exemplary embodiments, a suggestion to modify at least one parameter of the stimulation comprises a suggestion to modify at least one of, intensity, frequency, duration and location of the stimulation. In some embodiments, a suggestion to modify at least one parameter of the detection comprises a suggestion to modify at least one of, a position of at least one electrode, type of a measured signal and type of an input device for receiving input from the subject.

According to some exemplary embodiments, the generated suggestion is delivered, for example by the anesthesia monitoring device to at least one of a patient, and/or to a user of the anesthesia monitoring device, for example an anesthetist, a physician, a surgeon, or a caregiver. In some embodiments, the generated suggestion is delivered as a human detectable indication, for example an audio and/or a visual indication that is detectable by a human. Optionally, the generated suggestion is displayed on a screen, or is transmitted and displayed by a remote device, for example a device located outside a room of the monitored subject. In some embodiments, the generated suggestion is displayed next to the subject, for example next to the subject bed. Alternatively or additionally, the generated suggestion is displayed by a remote device, for example a computer, a handheld device and/or a display next to anesthesia information received from one or more patients.

According to some exemplary embodiments, the at least one parameter of anesthesia is optionally modified at block 114. In some embodiments, the at least one anesthesia parameter is optionally modified by a user of the anesthesia monitoring device, for example an anesthetist. Alternatively, the at least one anesthesia parameter is optionally modified automatically by the anesthesia monitoring device. In some embodiments, an indication, for example human detectable indication is generated and delivered once the at least one anesthesia parameter is optionally modified by the device. In some embodiments, the indication is delivered to a user of the device. Alternatively or additionally, the indication is delivered to a remote device located outside a room of the monitored subject, or to a user of a remote device.

According to some exemplary embodiments, the anesthesia effect is continuously monitored, for example following stimulation delivery at block 104. In some embodiments, the anesthesia effect is monitored in order to maintain a subject within a desired range of anesthesia effect, where the anesthesia affects desired tissues and/or organs of a body, while maintaining anesthesia in a level which is sufficient not to produce side effects or that the anesthesia level produces tolerable side effects in the subject.

According to some exemplary embodiments, an anesthesia effect for example anesthesia depth is determined by detecting transmission of sensory neural signals by specific neural fibers, for example following the stimulation. In some embodiments, different frequency ranges of the stimulating electric field induce generation and transmission of sensory neural signals through specific neural fibers. In some embodiments, a stimulating electric field with a frequency in a range of about 1800 Hz to about 4000 Hz, for example 1900 Hz to 2100 Hz, induce neural transmission of sensory signals via A-beta fibers. In some embodiments, a stimulating electric field with a frequency in a range of about 220 Hz to about 280 Hz, for example 240 Hz to 260 Hz, induce neural transmission of sensory signals via A-delta fibers. In some embodiments, a stimulating electric field with a frequency in a range of about 3 Hz to about 7 Hz, for example 4 Hz to 6 Hz, induce neural transmission of sensory signals via C fibers.

According to some exemplary embodiments, in order to determine an anesthesia depth, a stimulation, for example an electric field, is delivered with a frequency of about 5 Hz, at block 104. In some embodiments, if an ERP signal, for example a D-SSEP signal is received in the brain, for example by recording a D-SSEP signal from cortical or sub-cortical locations at block 106, then a system monitoring anesthesia detects that a c-fiber transmitting thermal sensation is not anesthetized, and therefore optionally a depth of anesthesia is not sufficient.

According to some exemplary embodiments, in order to determine an effect on A-delta fibers, which optionally deliver pinprick sensation, a stimulation, for example an electric field, is delivered with a frequency of about 250 Hz, at block 104.

According to some exemplary embodiments, in order to determine an effect on A-beta fibers, which optionally deliver touch sensation, a stimulation, for example an electric field, is delivered with a frequency higher than 1000 Hz, at block 104.

According to some exemplary embodiments, an anesthesia effect, for example an anesthesia effect distribution and/or anesthesia effect depth is determined by determining a relation between a signal recorded following delivery of stimulation, and at least one stored indication. Optionally, the anesthesia effect is determined by determining a relation between an indication of a signal recorded following delivery of stimulation stored in a memory, and at least one stored indication. In some embodiments, the at least one stored indication comprises at least one indication of a previously measured signal, measured from the same subject, or at least one indication of one or more previously measured signals, measured from at least one different subject. In some embodiments, the at least one stored indication and/or the indication of a signal recorded following delivery of stimulation is stored in a database, a cloud storage device, a server, or any remote device used for storage and/or for processing of data.

According to some exemplary embodiments, an anesthesia effect for example anesthesia effect distribution is determined by detecting changes in signals recorded following delivery of stimulation at two different stimulation locations, and/or at a similar stimulation location at different time points. In some embodiments, changes between a first signal recorded following a first stimulation and a second signal recorded following a second stimulation, indicate a distribution, for example axial distribution of the anesthesia effect on the tissue.

According to some exemplary embodiments, stimulation at block 104, detecting tissue response at block 106 and determining an anesthesia effect is repeated, every predetermined or varying time period of up to 2 minutes, for example up to 1 minute, up to 30 seconds, up to 10 seconds, up to 1 second or any intermediate, smaller or larger time duration between two repeated stimulations. In some embodiments, stimulation at block 104, detecting tissue response at block 106 and determining an anesthesia effect is repeated within an overall time period of at least 2 minutes, for example at least 5 minutes, at least 10 minutes or any intermediate smaller or larger value.

### Exemplary Anesthesia Effect

According to some exemplary embodiments, a subject, for example human subject is planned to undergo a treatment, for example a surgical procedure. In some embodiments, during the treatment, a target site needs to be anesthetized, for example to reduce or to block sensation at the target site. Alternatively or additionally, anesthetizing the target site reduces or blocks delivery of sensory information from the target site to the brain and/or to other regions or neuronal networks in the body. In some embodiments, sensation and sensory information comprises pain sensation and pain sensation information, respectively.

According to some exemplary embodiments, the anesthesia effect is based on axial distribution of the anesthesia along a longitudinal axis of the body, which optionally determines a height of a nerve block, for example a sensory nerve block. Alternatively or additionally, the anesthesia effect is based on anesthesia depth which determines the extent of nerve blockage and/or the extent of reduction and/or blockage of sensory information at different locations of the body. In some embodiments, the device and methods described herein are used to monitor the anesthesia effect or changes thereof over time, for example anesthesia height and/or anesthesia depth, optionally in relation to a pre-planned region. In some embodiments, monitoring the anesthesia effect allows, for example, to minimize or to avoid a risk of developing side effect of the anesthesia which are optionally associated with anesthesia height which is higher or lower than a desired anesthesia axial height, and/or anesthesia depth which is larger than a desired anesthesia depth.

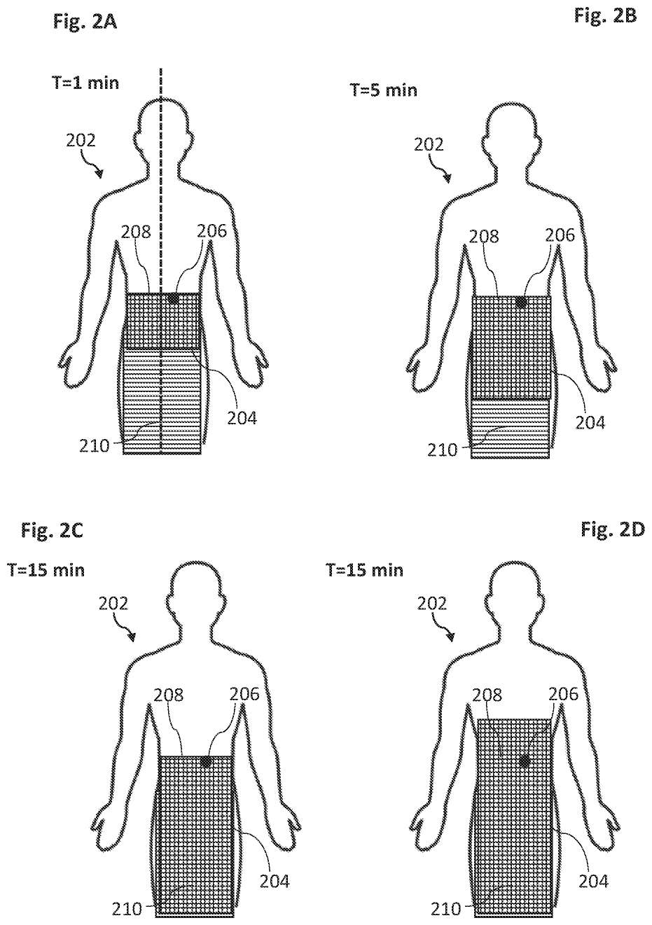

Reference is now made to FIGS. 2A-2C depicting changes in the axial distribution of anesthesia in a body of a human subject, according to some exemplary embodiments of the invention.

According to some exemplary embodiments, subject 202 is anesthetized, for example with one or more anesthetic compounds suitable for regional anesthesia, in order to reduce sensation and/or sensation information delivery within an affected region 204. In some embodiments, the affected region is selected according to a planned treatment or a planned procedure, for example a planned surgical procedure, and/or childbirth. Optionally, the affected region is selected while delivering a baby during a childbirth.

According to some exemplary embodiments, anesthesia, for example one or more anesthetic compounds are introduced into the body of the subject 202, for example via one or more body entry sites, for example at an injection site 206. In some embodiments, the anesthesia is introduced into the body at a predetermined rate of administration and/or at a predetermined dose of the one or more anesthetic compounds. In some embodiments, the injection site 206 is located on the back of the subject 202. In some embodiments, the injection site location, the predetermined dose and/or the predetermined administration rate is selected according to at least one of the treatment and/or procedure duration, a clinical condition of the subject 202 and the type and/or characteristics of the treatment and/or procedure.

According to some exemplary embodiments, for example as shown in FIG. 2A, one or more anesthetic compounds are administered to a subject body via at least one injection site 206, for example an administration site or an infusion site. In some embodiments, administering the one or more anesthetic compounds leads to a regional anesthesia effect between the injection site 206 and the feet of the subject.

According to some exemplary embodiments, for example as shown in FIGS. 2A-2C, the regional anesthesia affects body regions between the injection site 206 and the feet of the subject, for example regions below the injection site, a depth 204 of the regional anesthesia changes with time.

According to some exemplary embodiments, for example as shown in FIG. 2D, the anesthesia effect expands with time, to an axial distribution level 210 which is higher than a target anesthesia height, and is located between the injection site 206 and a head of the subject. In some embodiments, the axial distribution level is higher, for example proximal relative to the injection site 206. In some embodiments, a higher anesthesia level leads to blockage of nerves innervating the respiratory system, for example nerves innervating muscles of the respiratory system, which may lead to at least one of a respiratory failure, low blood pressure, dizziness, fainting. In some embodiments, in FIG. 2D, the anesthesia affects regions above and below injection site 206.

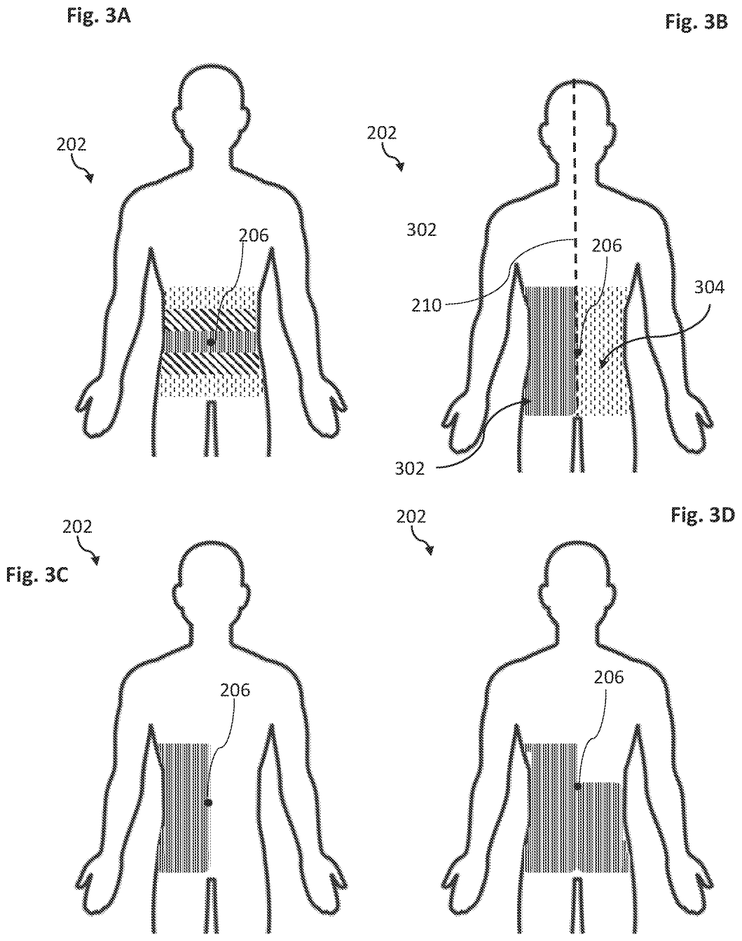

Reference is now made to FIGS. 3A-3D depicting variations in anesthesia depth between different regions of the body, according to some exemplary embodiments of the invention.

According to some exemplary embodiments, for example as shown in FIG. 3A, the anesthesia depth is uneven, and is higher in areas closer to the anesthesia administration site 206, relative to areas that are located at a distance from the site 206.

According to some exemplary embodiments, for example as shown in FIG. 3B, the anesthesia depth level is sufficient in a first lateral part of a human body relative to a lower non-sufficient anesthesia depth level in a second lateral part of the human body. In some embodiments, for example as shown in FIG. 3C, a lateral distribution of the anesthesia effect, for example an anesthesia depth, is unilateral, and there is not anesthesia effect in one side of the body.

According to some exemplary embodiments, for example as shown in FIG. 3D, there is a difference in anesthesia height between different sides of the body.

### Exemplary Monitoring of Local Anesthesia Effect

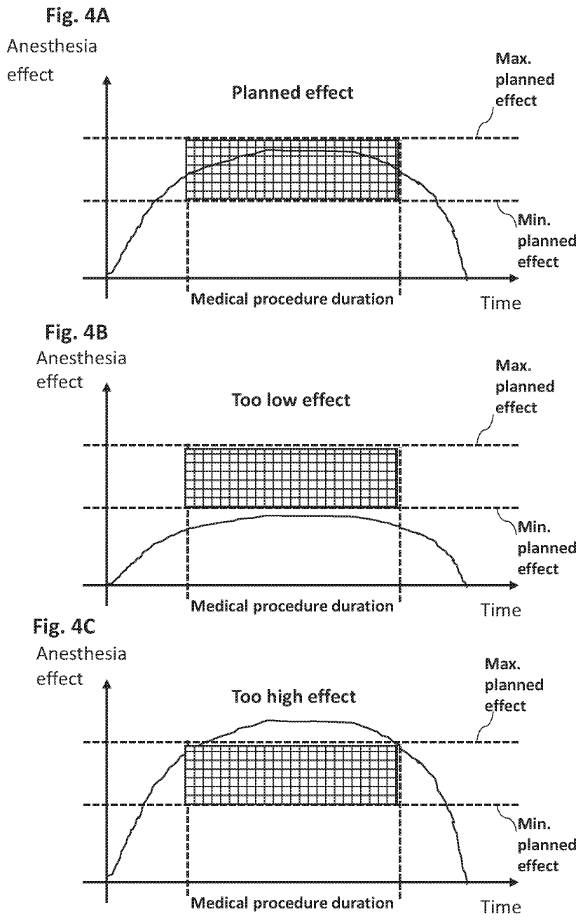

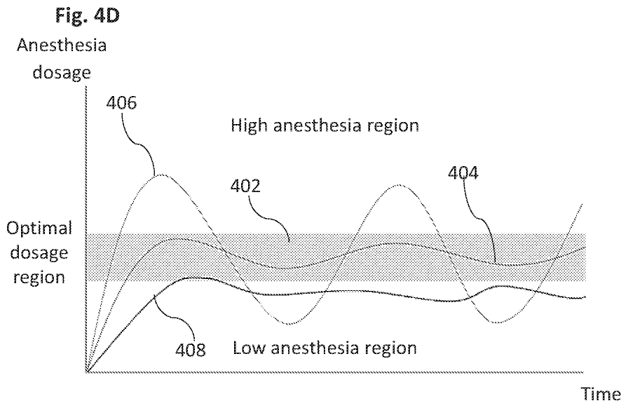

According to some exemplary embodiments, an effect of anesthesia, for example neuraxial anesthesia, is monitored at a measurement site. In some embodiments, the measurement site comprises a body region which is related to a treatment and/or to a medical procedure, for example a body region that is affected during the treatment and/or the medical procedure. Alternatively or additionally, the measurement site is a location in the body, where the measured anesthesia indicates an effect of anesthesia on a target site, for example a remote target site that is affected by a medical treatment and/or by a medical procedure. In some embodiments, monitoring local effect of anesthesia at the measurement site is important, for example to make sure that the anesthesia effect is a planned effect, for example as expected according to a predetermined plan, and that the local effect is not higher relative to the predetermined plan. Reference is now made to FIGS. 4A-4C depicting monitoring a local anesthesia effect, according to some exemplary embodiments of the invention.

According to some exemplary embodiments, an effect of anesthesia on a target site is monitored. In some embodiments, the target site comprises a body region that is optionally affected by and/or during a medical procedure, or a body region that is related to the medical procedure. In some embodiments, anesthesia administration is optionally controlled, for example, in order to achieve and maintain a planned anesthesia effect at the target site, for example during a medical procedure, and/or while delivering a baby during childbirth. In some embodiments, for example as shown in FIG. 4A, a planned anesthesia effect is an anesthesia effect that is between a minimal level of anesthesia effect and a maximal level of anesthesia effect.

According to some exemplary embodiments, the minimal and/or the maximal levels of anesthesia effect are predetermined, for example are determined prior to the beginning of a medical procedure or prior to the delivery of anesthesia. In some embodiments, the minimal and/or the maximal levels of anesthesia effect are optionally modified during the medical procedure and/or when the medical procedure changes, for example to treat a different tissues of the body.

According to some exemplary embodiments, for example as shown in FIG. 4B, an anesthesia effect which is lower than the minimal planned effect, is not a sufficient anesthesia effect for a selected medical procedure. In some embodiments, if the anesthesia effect is not a sufficient effect, then optionally, sensation level at a target site and/or delivery of sensory information from a target site is higher than a maximal level. In some embodiments, if a system for delivery of anesthesia, for example a system for delivery of neuraxial anesthesia, detects that a measured anesthesia effect at a target site and/or at a measurement site is lower than a planned effect then at least one parameter of anesthesia administration is modified. In some embodiments, if the system detects that a measured anesthesia effect at a target site and/or at a measurement site is lower than a planned effect then the anesthesia administration rate is increased and/or a dose of the administered anesthesia is increased.

According to some exemplary embodiments, for example as shown in FIG. 4C, an anesthesia effect which is higher than a maximal planned effect is an over effect, which optionally affects unwanted organs and/or tissue. In some embodiments, an over effect optionally leads to side effects, for example loss of sensation and/or paralysis of body parts that are not related to the medical procedure. In some embodiments, if system for delivery of anesthesia detects that an anesthesia effect at a target site or at a measurement site is an over effect, at least one parameter of the anesthesia administration is modified. In some embodiments, if an over effect is detected, the anesthesia administration rate and/or the anesthesia dose is reduced. Alternatively, if an over effect is detected, the anesthesia administration is stopped and/or an additional supporting treatment is provided such as: oxygen administration, vasoactive drugs administration, for example to raise blood pressure.

According to some exemplary embodiments, the system and/or method described herein are used to monitor the effect of anesthesia, for example neuraxial anesthesia, relative to at least one parameter of the anesthesia administration, for example dosage and/or rate of administration. In some embodiments, monitoring the effect of anesthesia allows to maintain the at least one parameter of anesthesia administration within a range that allows optimal anesthesia effect.

According to some exemplary embodiments, for example as shown in FIG. 4D, a dosage of anesthesia administered to the body, as represented by line 404, is maintained within an optimal dosage region 402, over time, for example during a surgical procedure or during a treatment. In some embodiments, since the anesthesia is administered continuously or intermittently over time, a dosage of the anesthesia fluctuates over time. In some embodiments, the method and device described herein are used to maintain the anesthesia dosage including the fluctuations of the anesthesia dosage within the optimal dosage region 402. Alternatively, the method and the device described herein are used to provide information to a physician with regard to anesthesia effect. Optionally, the physician decided whether or not to change the anesthesia administration based on the provided information

In some embodiments, changes in dosage levels to dosage levels higher than an optimal range of dosage levels, as represented by line 406, for example to dosage levels within a high anesthesia region, may lead to unwanted side effects which comprise decrease blood pressure, distribution of the anesthesia effect to regions higher than the administration site, neural blockage of the chest region, etc.

In some embodiments, changes in dosage levels or dosage levels lower than an optimal range of dosage levels, as represented by line 408, may lead to insufficient neural blockage and pain sensation by the subject.

### Exemplary System

According to some exemplary embodiments, a system for delivery of anesthesia, for example neuraxial anesthesia is configured to receive information indicating an effect of the anesthesia. In some embodiments, the received information optionally indicates the anesthesia effect at one or more measurement sites. In some embodiments, based on the anesthesia effect indication, the system determines a distribution of the anesthesia effect in the body, for example the anesthesia height and/or the depth of the anesthesia and/or a location of an anesthetized region. Alternatively or additionally, based on the anesthesia effect indication, the system optionally determines an effect of the anesthesia on a target site, for example a body region or tissues that are related to and/or that are affected by a medical procedure, for example a treatment, a surgical procedure and/or while delivering a baby during a childbirth. Alternatively or additionally, based on the anesthesia effect indication, the system optionally modifies or recommends to modify one or more parameters of the anesthesia administration, for example anesthesia delivery, for example delivery rate, anesthesia dosage, injection site, type of anesthetizing compounds, and/or ratio between anesthetizing compounds.

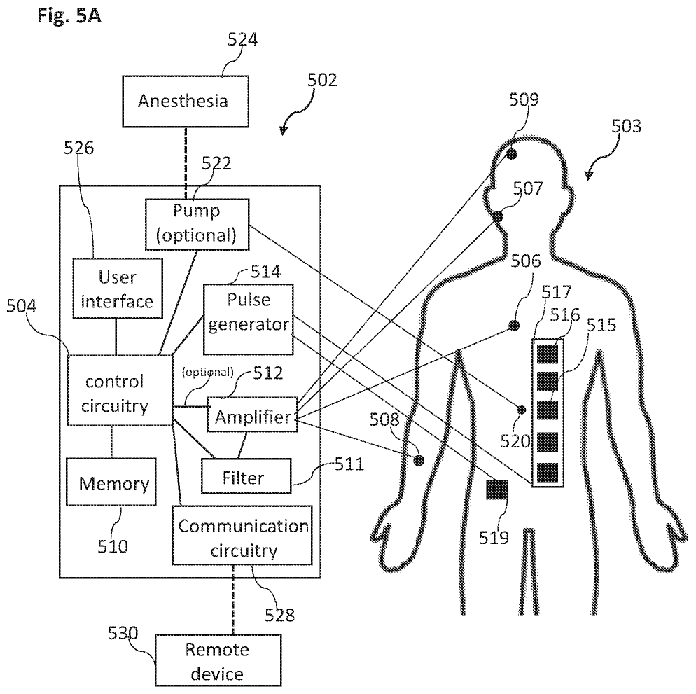

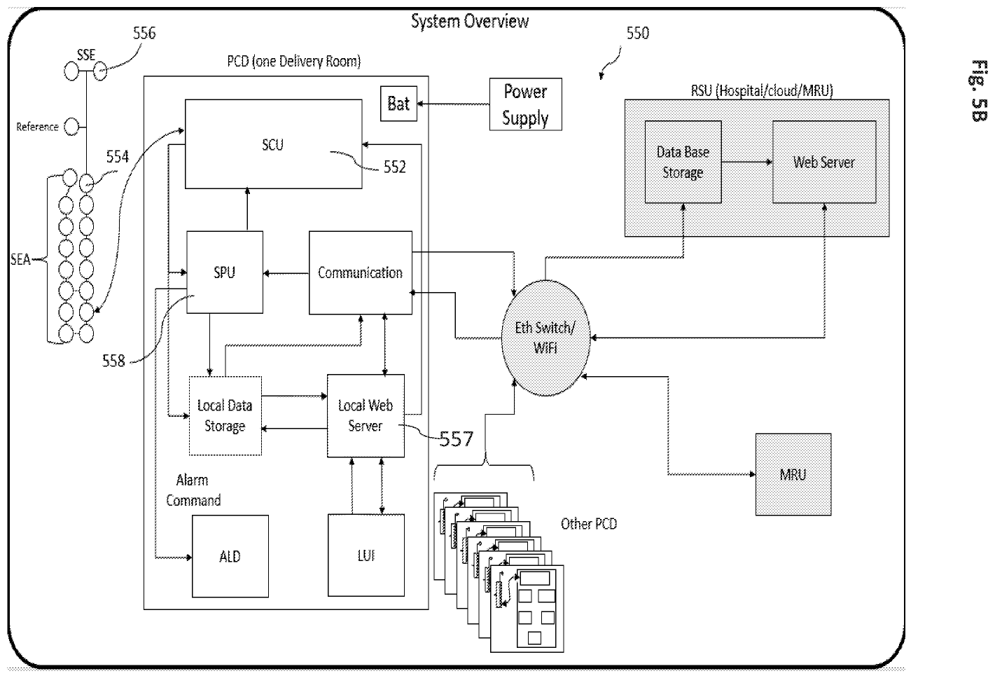

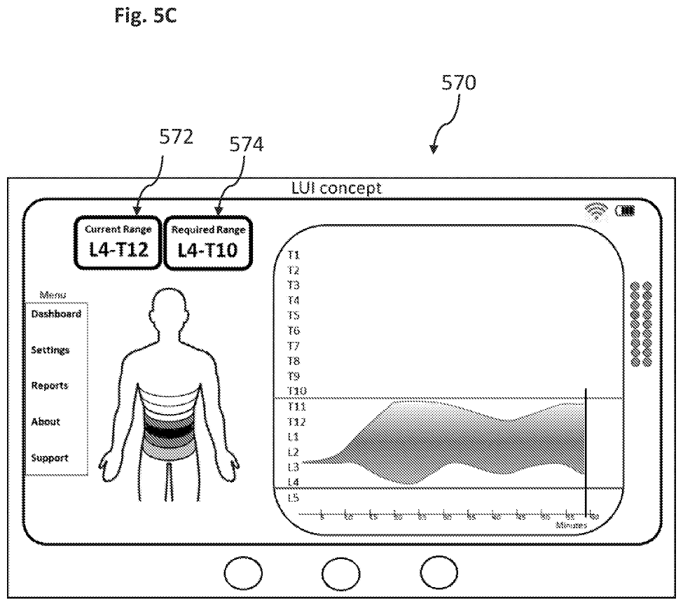

Reference is now made to FIG. 5A depicting a system for monitoring an anesthesia effect, according to some exemplary embodiments of the invention.

According to some exemplary embodiments, a system for anesthesia effect monitoring, for example system 502 comprises a control circuitry 504 and one or more sensors, for example at least one sensing electrode. In some embodiments, the at least one sensing electrode comprises sensing electrodes 506, 507, 508 and 509. In some embodiments, the sensing electrodes 506 and 508 are configured to sense and deliver signals to the control circuitry. In some embodiments, the at least one sensing electrode comprises at least one of a movement sensor, a temperature sensor, and a sensor for sensing at least one electrical parameter of the tissue, for example electrical conductivity, electric potentials, and/or impedance. In some embodiments, the at least one sensing electrode is introduced into the body, for example through an anatomical opening of the body, or through a surgical opening formed in the body. Optionally, the at least one sensing electrode is introduced through the skin surface into the body, for example into a muscle. Alternatively or additionally, the at least one sensing electrode is positioned on the skin, for example attached to the skin surface.

In some embodiments, the at least one electrode, for example electrodes 506 and/or 508 comprises an EMG electrode configured to record electrical activity produced by muscles. In some embodiments, the EMG electrode is attached to a skin surface at a specific measurement site, for example above a muscle. In some embodiments, the EMG electrode is attached to a skin surface of a back, abdomen, chest, limb, face, and/or neck. In some embodiments, the EMG electrode measures a signal in a range for example up to 200 micro volts (mV), for example up to 100 micro volts (mV), for example up to 20 micro volts (mV), for example up to 10 micro volts (mV), for example between 20-3000 micro volts (mV), for example 20-1000 mV. 100-1000 mV, 500-2000 mV, 1000-3000 mV or any intermediate, smaller or larger range of values. In some embodiments, the EMG electrode measures a signal in a range between 1-10 mV, for example a signal in a range between 1-5 mV, a signal in a range between 3-7 mV, a signal in a range between 4-10 mV, or any intermediate, smaller or larger range of values.

According to some exemplary embodiments, when measuring signals, for example ERP, signals in cortical and/or in sub-cortical locations in response to stimulation, measurement is performed in up to 300 milliseconds (ms) after the end of the stimulation, for example measurement is performed in up to 200 milliseconds (ms) after the end of the stimulation, for example measurement is performed in up to 100 milliseconds (ms) after the end of the stimulation, for example measurement is performed in up to 50 milliseconds (ms) after the end of the stimulation, for example measurement is performed in up to 20 milliseconds (ms) after the end of the stimulation, for example measurement is performed in up to 15 milliseconds (ms) after the end of the stimulation or any intermediate, smaller or larger value. In some embodiments, the measured signals comprise at least one of, electric signals, potentials. Neural activity signals, and SSEP, for example D-SSEP signals.

According to some exemplary embodiments, ERP signals, for example SSEP signals are measured after 0 milliseconds, after at least one 1 millisecond, after at least 5 milliseconds, after 10 milliseconds, after 15 milliseconds or any intermediate, smaller or larger value following the delivery of stimulation, for example following an initiation of the delivery or the end of the delivery of the stimulation.

In some embodiments, the at least one electrode, for example electrode 509 comprises an EEG electrode configured to record electrical signals from the brain indicating brain activity. In some embodiments, the EEG electrode is positioned on a scalp of the subject. In some embodiments, the at least one electrode, for example an EEG electrode is configured to sense and record ERPs, for example somatosensory evoked potentials (SSEPs), from brain locations and/or from different nerves. Alternatively or additionally, the at least one electrode is positioned and configured to record neural conduction in nerves directed to and/or from the brain. In some embodiments, the at least one sensing electrode is positioned in at least one of, a body region of the subject, a leg, arm, chest, back, abdomen, a scalp of a subject, face, forehead, neck, nape, and/or on a head of the subject above a hairline or below a hairline. In some embodiments, the at least one sensing electrode measure at least one signal in a range of 1-20 micro-Volts, for example 1-10 micro-Volts, 3-6 micro-Volts, 5-15 micro-Volts, 10-20 micro-Volts or any intermediate, smaller or larger range of values. The at least one sensing electrode is positioned on a head, on a scalp, behind one or both cars, and/or on the nape, and/or the back. In some embodiments, the at least one sensing electrode records one or more electrical signals from cortical and/or subcortical regions. Alternatively or additionally, the at least one electrode records neuronal activity and/or neural conductivity outside the brain, for example outside the central nervous system (CNS).

According to some exemplary embodiments, the at least one sensing electrode, for example sensing electrode 506 is attached to a muscle of a subject, for example to a surface of the skin above a muscle. In some embodiments, the at least one sensing electrode comprises an electromyography (EMG) electrode. In some embodiments, the at least one sensing electrode is configured to sense muscle contraction, for example an involuntary muscle contraction, in response to a stimulation delivered through at least one of stimulating electrode, for example a stimulator. In some embodiments, the at least one sensing electrode is attached to the back skin above the Trapezius muscle, for example between the Scapulae. Alternatively or additionally, the at least one electrode is attached to the skin surface of the face, nape on a scalp of a subject and/or to the subject back. In some embodiments, the at least one sensing electrode comprises a dry electrode or a wet electrode.

In some embodiments, at least one additional sensing electrode is configured to sense and deliver information regarding contraction of at least one additional muscle, for example to allow noise reduction by the control circuitry 504. Alternatively or additionally, noise reduction from the signal received from the at least one sensing electrode will be performed using one or more algorithms stored in the memory 510, optionally by modulating the signal. In some embodiments, when measuring EMG, noise reduction is performed by measuring EMG signals from at least one additional reference electrode. Alternatively or additionally, when measuring EEG signals, noise reduction is performed by measuring EEG signals from at least one additional reference electrode.

According to some exemplary embodiments, the one or more sensors, comprises at least one sensor configured to be placed in contact with the subject body, for example the at least one sensing electrode. Alternatively, the one or more sensors comprises at least one sensor positioned at a distance from the body of the subject, for example a thermal sensor. In some embodiments, the thermal sensor comprises a thermal camera. In some embodiments, the one or more sensors is configured to be positioned at a distance larger than 10 cm, for example at a distance larger than 20 cm, larger than 30 cm or any intermediate, shorter or larger distance from the body of the subject. In some embodiments, the one or more sensors, for example the thermal camera is positioned at a distance between 5 cm and 5 meters from the subject, for example at a distance between 5 cm and 1 meter, at a distance between 20 cm and 1.5 meters or any intermediate, smaller or larger range of distances from the subject body.

According to some exemplary embodiments, the control circuitry 504 is configured, for example programmed, to determine an anesthesia effect and/or to determine a distribution of the anesthesia effect, based on the signals received from the sensing electrodes 506 and 508. Alternatively or additionally, the control circuitry 504 is configured, for example programmed, to determine an anesthesia effect and/or to determine a distribution of the anesthesia effect, based on the detected signals and a-prior stored data. In some embodiments, the control circuitry is configured to determine the anesthesia effect using one or more algorithms, lookup tables, and/or indications stored in a memory, for example memory 510.

According to some exemplary embodiments, the memory of the system is part of the remote device 530, which is in communication with the control circuitry 504. In some embodiments, the remote device comprises a server, for example a local server of a hospital or a medical facility, or a cloud-based server. In some embodiments, at least some of the information transferred between the control circuitry 504 and the remote device 530 is encrypted for example using an Advanced Encryption Standard (AES) algorithm. In some embodiments, at least some of the information stored in the remote device 530 is encrypted for example using an Advanced Encryption Standard (AES) algorithm.

According to some exemplary embodiments, the control circuitry 504 is configured to differentiate between a tissue that is under an effect of administered anesthesia and a tissue that is not affected by the administered anesthesia, optionally using an algorithm, for example an algorithmic classifier. Alternatively or additionally, the control circuitry 504 is configured to differentiate between tissues with different levels of anesthesia depth optionally using an algorithm, for example an algorithmic classifier. In some embodiments, the control circuitry 504 differentiates between the different tissues based on differences in signals recorded from each tissue in response to one or more stimulations, optionally using an algorithm. In some embodiments, the algorithm, for example an algorithmic classifier is stored in memory 510 and/or in a memory of the remote device 530. Optionally, the remote device is used to at least one of, differentiate between a tissue that is under an effect of administered anesthesia and a tissue that is not affected by the administered anesthesia, to differentiate between tissues with different levels of anesthesia depth.

According to some exemplary embodiments, the system 502, for example the control circuitry 504, is configured to modify at least one parameter of the stimulation based on the differentiation between the different tissues, for example based on tissue classification, and/or based on a clinical scenario, for example a planned procedure.

According to some exemplary embodiments, the memory 510 stores two or more algorithms, for example two or more algorithmic classifiers, each including a different classifying model. In some embodiments, the system 502, for example the control circuitry 504 uses a specific algorithmic classifier for processing the signals received from the subject and/or stored data, and can optionally shift to a different algorithmic classifier stored in the memory 510. It should be understood that the processing described herein performed by the control circuitry 504 can be alternatively or additionally performed by the remote device, for example remote device 530, using data stored in the memory 510 or in the remote device 530.

According to some exemplary embodiments, the system 502 comprises at least one amplifier, for example amplifier 512. In some embodiments, the amplifier 512 is configured to amplify signals, for example electric signals received from the sensing electrodes 506 and 508. In some embodiments, the amplifier 512 comprises a differential amplifier, configured to generate a differential signal from signals received from two or more sources, for example from two or more sensing electrodes. Potential advantage of using a differential signal may be to reduce noise from the received signals, for example prior to processing and/or analysis performed by the control circuitry 504. Alternatively or additionally, the amplifier 512 comprises a Low Noise Amplifier, configured to amplify the desired signal while decaying the thermal noise and other Interferences.

According to some exemplary embodiments, the system 502 comprises at least one filter, for example filter 511 configured to filter a signal received by the amplifier and/or a signal received from at least one sensing electrode. In some embodiments, the filter 511 comprises a low pass filter, a high pass filter, a band pass filter, a Kalman filter, a Notch filter and/or a surface acoustic wave (SAW) filter.

According to some exemplary embodiments, the filter 511 is configured to filter a signal received from at least one sensing electrode to receive a signal within a frequency range of 1-4000 Hz, for example within a range of 1-2000 Hz, 1000-3000 Hz, 2000-4000 Hz or any intermediate, smaller or larger range of values. Alternatively, the filter 511 is configured to filter signals to receive a signal within a frequency range of 1-4000 Hz, for example within a range of 1-2000 Hz, 1000-3000 Hz, 2000-4000 Hz or any intermediate, smaller or larger range of values. Optionally, the filter 511 filters signals using a Notch filter.

According to some exemplary embodiments, the system 502 comprises at least one pulse generator, for example pulse generator 514. In some embodiments, the pulse generator 514 is configured to generate pulses of energy, for example electric field pulses, optionally in response to signals received from the control circuitry 504. In some embodiments, the pulse generator 514 is connectable to one or more electrodes placed in contact with a body of the subject 503. In some embodiments, the pulse generator 514 generates electric field pulses and deliver the pulses to the subject body through the one or more electrodes.

According to some exemplary embodiments, the control circuitry 504 is programmed to signal, for example to activate, the pulse generator 514 to generate the electric field pulses, optionally at least one electric field pulse, based on indications stored in the memory 510. In some embodiments, the control circuitry 504 is programmed to signal the pulse generator 514 to generate the electric field pulses, optionally at least one electric field pulse, in a timed relationship, for example prior and/or during with receiving the signals from the at least one sensing electrode, for example sensing electrodes 506 and 508. Alternatively, or additionally, the control circuitry 504 is programmed to receive signals from the at least one sensing electrode, for example sensing electrodes 506 and 508, in a timed relationship, for example during and/or following the generation of at least one electric field pulse by the pulse generator 514.

According to some exemplary embodiments, the pulse generator 514 comprises an electric pulse generator. In some embodiments, the pulse generator 514 is configured to generate and deliver pulses, for example electric field pulses, in fixed or varying intervals, for example every at least 180 milliseconds, for example every at least 5 seconds, for example every at least 30 seconds, every at least 1 minute, every at least 5 minutes, every at least 10 minutes, every at least 15 minutes or every any intermediate, smaller or larger time period. In some embodiments, the pulse generator is configured to deliver the pulses, for example the electric field pulses in intervals, for a time period of one or more hours, or one or more days.

According to some exemplary embodiments, the pulse generator 514 is configured to generate an electric field in frequencies of at least 0.1 Hz, for example at least 10 Hz, at least 50 Hz, at least 100 Hz, at least 250 Hz, at least 2000 Hz or any intermediate, smaller or larger frequency.

According to some exemplary embodiments, the pulse generator 514 is configured to generate pulses of an electric field with intensity values sufficient to evoke a sensory response, but are lower than a pain sensation threshold in a subject. In some embodiments, the intensity values are in a range between 0.5-40 mA, for example 0.5-10 mA, 5-20 mA, 10-30 mA, 20-40 mA or any intermediate, smaller or larger range of values. In some embodiments, the intensity values are in a range of 0.5-20 mA, for example 2.5-10 mA, 2.5-8 mA, 2.5-5 mA or any intermediate, smaller or larger range of values. In some embodiments, the intensity values are up to 40 mA, for example up to 30 mA, up to 10 mA or any intermediate, smaller or larger value.

In some embodiments, the pulse generator is configured to generate electric field pulses with parameter values, for example frequency, current and/or timing values, that are sufficient to activate neural circuits that are similar to neural circuits activated by thermal stimulation, contact stimulation, pinch and/or puncturing stimulation in the subject.

In some embodiments, the pulse generator is configured to generate electric field pulses continuously, intermittently, in a repetitive stimulation pattern, or randomly. In some embodiments, a delay time window between consecutive stimulations is fixed or varies. In some embodiments, the stimulation pulses are delivered in frequencies in a range between 0.1 Hz and 10 Hz, for example 1-5 Hz, 1-8 Hz, 3-8 Hz, 5-10 Hz or any intermediate, smaller or larger range of values.

In some embodiments, the stimulation pulses are delivered in frequencies in a range between 0.1 Hz-2000 Hz, for example 1-5 Hz, 1-8 Hz, 3-8 Hz, 5-10 Hz, 10-50 Hz, 100-250 Hz, 1800-2500 Hz or any intermediate, smaller or larger range of values.

According to some exemplary embodiments, the system 502 comprises one or stimulators optionally arranged in an array, for example stimulating electrodes 515 and 516, optionally arranged in an array 517, for example an axial array. In some embodiments, the stimulating electrodes 516 are connected, for example electrically connected to the pulse generator 514. In some embodiments, the stimulating electrodes 516 or the array 517 are attached to the subject body, optionally to a back of the subject body. In some embodiments, the stimulating electrodes 516 array is attached, optionally using adhesive, to the skin of the subject body. In some embodiments, the stimulating electrodes 516 or array 517 are optionally attached to the skin of the subject back. Alternatively, the stimulating electrodes 516 or array 517 are attached to at least one of a chest, abdomen, limb or any body part of the subject body.

According to some exemplary embodiments, the one or more stimulating electrodes, for example stimulating electrodes 516 are attached to the body of the subject at a predetermined distance from a target anesthesia site surrounding an injection or an infusion site 520, or a site that needs to be anesthetized. Alternatively, the one or more stimulating electrodes, for example stimulating electrodes 516 are attached to the body of the subject at the target site. In some embodiments, the one or more stimulating electrodes comprise at least one electrode, for example, electrode 519, which is optionally attached to different parts of the body and is used, for example, when determining an effect of local anesthesia or peripheral anesthesia.

According to some exemplary embodiments, the one or more stimulating electrodes comprises at least one stimulating electrode, for example at least 2 stimulating at least 5 stimulating electrodes, at least 7 stimulating electrodes, at least 9 stimulating electrodes or any intermediate, smaller or larger number of electrodes. In some embodiments, the stimulating electrodes are arranged in at least one array, attached to a back of the subject or to any part of the body. In some embodiments, the at least one array is an axial array attached to a back of a subject along a longitudinal axis of the body and/or along an axis that passes through to or more dermatomes. In some embodiments, a central electrode, for example electrode 515 in the axial array is positioned at a same height on the back as an anesthesia infusion site 520. As used herein, the term central electrode refers to any electrode that is not positioned at an end of the electrode array. Alternatively, any electrode of the array can be positioned at the same height as the infusion site. In some embodiments, the electrodes array is attached to the back of a subject near a midline, of the subject back which is parallel to a longitudinal axis of the body, for example at a distance of up to 3 cm, up to 10 cm, up to 15 cm or any intermediate, smaller or larger distance from the spinal cord of the subject.

According to some exemplary embodiments, the electrodes array is flat, for example planar. Additionally, the electrodes array is thin, for example has a thickness of up to 30 mm, for example up to 20 mm, up to 10 mm, for example up to 5 mm, for example up to 1 mm or any intermediate, smaller or larger thickness. In some embodiments, the at least one stimulating electrode is shaped and sized to deliver a stimulation with a current density in a range between 0.1-20 microampere per square mm (μA/mm²), for example in a range between 1-10 (μA/mm²), for example in a range between 10-20 (μA/mm²), for example in a range between 5-9.5 (μA/mm²), for example in a range between 10-13.8 (μA/mm²), or any intermediate, smaller or larger range of values.

In some embodiments, the array of electrodes is attached to the back of the subject by an adhesive. In some embodiments, each of the electrodes of the electrode array is separately electrically connected to the system 502, for example to the pulse generator 514.

According to some exemplary embodiments, the control circuitry 504 signals the pulse generator 514 to generate and deliver one or more pulses of energy, for example electric energy, to one or more electrodes of the stimulating electrodes 516 array. In some embodiments, the pulse generator 514 delivers energy pulses to one or more electrodes in the array in a predetermined sequence, for example in a sequence stored in the memory. In some embodiments, the pulse generator 514 delivers energy pulses first to at least one electrode located proximal, for example closer to at least one anesthesia infusion site, for example injection site 520, and then to at least one more distal electrode located at a distance from the injection site 520. In some embodiments, the control circuitry 504 determines the anesthesia effect, for example anesthesia depth and/or height based on signals from the at least one sensing electrode, for example sensing electrodes 506 and 508 received after each stimulation or after one or more stimulations. In some embodiments, the control circuitry 504 is operable to receive signals from at least one sensing electrode which are associated with the delivered stimulation signals. In some embodiments, the control circuitry 504 is operable to modify at least one parameter of the stimulation according to the determined effect.

According to some exemplary embodiments, the system 502 comprises at least one pump, for example pump 522. Alternatively, the system comprises at least one actuator, controlled by the control circuitry 504. In some embodiments, the actuator is functionally connected to at least one external pump, and allows to control the activation of an external pump based on signals received from the control circuitry 504.

According to some exemplary embodiments, the pump is configured to advance at least one anesthetic at a selected rate, form an anesthetic containing chamber 524 to at least one infusion site, for example infusion site 520. In some embodiments, the pump 522 advances the at least one anesthetic to the infusion site 520 within at least one tube coupled to the pump 522. In some embodiments, the pump 522 advances the at least one anesthetic based on signals received from control circuitry 504, and/or according to indications, for example indications of one or more treatment protocols stored in the memory 510.

According to some exemplary embodiments, the control circuitry 504 controls the administration rate of the at least one anesthetic, by optionally controlling the operation of the pump 522. In some embodiments, the control circuitry 504 controls the operation of the pump 522, according to the determined anesthesia effect, for example according to the anesthesia height and/or according to the anesthesia depth. In some embodiments, if the anesthesia effect is optionally higher than a predetermined value, then the control circuitry 504 signals the pump 522 to decrease a rate of anesthetic administration, for example to decrease a rate of anesthetic advancement from the chamber 524 to the infusion site 522. Alternatively, the control circuitry 504 signals the pump 522 to stop the anesthetic administration.

According to some exemplary embodiments, if the anesthesia effect is optionally lower than a predetermined value, then the control circuitry 504 signals the pump 522 to increase a rate of anesthesia administration, for example to increase a rate of anesthesia advancement from the chamber 524 to the infusion site 522. Alternatively or additionally, the control circuitry signals a pulse generator to modify at least one parameter of the stimulation signal.

According to some exemplary embodiments, the predetermined value is a dynamic value that changes based on at least one of the signals received from the at least one sensing electrode, measured signals that indicate a clinical state of the subject, medical procedure, type of treatment, and/or drug regime of the subject.

According to some exemplary embodiments, the system 502 comprises at least one user interface, for example user interface 526. In some embodiments, the user interface 526 is configured to generate a human detectable indication, for example an audio and/or a visual indication that can be detected by a human subject. In some embodiments, the control circuitry 504 signals the user interface 526 to generate the human detectable indication if the rate of anesthesia administration is changed, optionally if an operation of the pump 522 is modified. Alternatively or additionally, the control circuitry 504 signals the user interface 526 to generate the human detectable indication with information regarding to the determined anesthesia effect and/or with information regarding the determined distribution of the anesthesia effect. In some embodiments, the user interface 526 is configured to deliver one or more indications to a user with recommendations to at least one of, modify the anesthesia dose and/or composition, to modify the anesthesia administration rate, to stop anesthesia administration, to modify an operation of at least one pump controlling the anesthesia administration, to change an anesthesia infusion site, to modify at least one parameter of a treatment and/or a medical procedure, to modify a position of the at least one sensing electrode, to modify a position of at least one stimulating electrode and to modify a position of an anesthesia infusion site. Optionally, the user interface 526 delivers the one or more indications to the user based on the determined anesthesia effect and/or the determined anesthesia effect distribution.

According to some exemplary embodiments, the user interface 526, optionally comprising a display, is configured to display data regarding more than on patient. In some embodiments, the user interface 526 comprises an alarm management scheme configured to generate and deliver alarm indications, for example according to a severity level of each patient.

According to some exemplary embodiments, the user interface 526 is configured to receive input from a user of the system 502. In some embodiments, the received user input comprises at least one of a change in anesthesia administration, a change in the operation of the pump 522, a change in a setup of the system 502, a change in a method for determining the anesthesia effect and/or the anesthesia effect distribution.

According to some exemplary embodiments, a user of the system, for example a physician will insert information into the system, optionally using the user interface 526, that includes at least one of, demographic details of the subject, for example age, BMI, weight, height, gender, a location of the infusion site 520 into which an epidural catheter is inserted, an insertion depth of the epidural catheter, location of at least one sensory electrode, location of at least one stimulation electrode. In some embodiments, location of at least one stimulating electrode and/or a location of at least one sensory electrode optionally comprises a height of the location on a back of a subject, for example a height relative to a reference location or relative to spinal cord spines or vertebrae.

According to some exemplary embodiments, a display of the user interface 526 will display to a user of the system 502 at least one of a desired anesthesia effect distribution, a desired height of anesthesia, a desired depth of anesthesia, current anesthesia height, current anesthesia depth, rate of anesthesia administration, total amount of anesthesia administered to the subject, anesthesia distribution, current state of anesthesia, axial height of anesthesia, location of at least one stimulation electrode, and/or location of at least one sensing electrode. In some embodiments, the system will display to a user the amount of drug, for example the amount of anesthesia, that needs to be added to the subject for different medical procedures and/or when changing a medical procedure. In some embodiments, the user interface 526 will allow a user to accept suggestions of the system to modify anesthesia administration, or to override the system suggestions and to manually operate the pump or an actuator controlling anesthesia administration.

According to some exemplary embodiments, the user interface 526 comprises a display. Alternatively or additionally, the user interface 526 comprises at least one button and/or at least one keyboard.

According to some exemplary embodiments, the system 502, for example control circuitry 504 is configured to detect that a determined effect of anesthesia is not according to a planned anesthesia effect, for example by determining a relation between the determined anesthesia effect and a planned anesthesia effect or indications thereof stored in the memory 510. Optionally, the control circuitry calculates a relation between the determined anesthesia effect and the planned anesthesia effect or indications thereof. In some embodiments, the control circuitry 504 signals the user interface 526 to generate a human detectable indication, for example an alert signal, indicating the determined relation.

According to some exemplary embodiments, the control circuitry 504 is configured to detect that a determined depth of anesthesia is not according to a planned anesthesia depth, and to signal the user interface 526 to generate an alert signal indicating the relation between the determined anesthesia depth and the planned anesthesia depth.

According to some exemplary embodiments, the control circuitry 504 is configured to detect that a distribution, for example axial distribution, of an anesthesia effect is not according to a planned axial distribution, and to signal the user interface 526 to generate and alert signal indication the relation between the determined axial distribution and the planned axial distribution.

According to some exemplary embodiments, the control circuitry 504 is configured to signal said user interface 526 to generate an alarm indication in different predetermined scenarios, for example if the anesthesia effect distribution is slower that a target distribution time and/or has a distribution range that is smaller than a target, for example a desired, distribution range, optionally allowing to identify a mispositioned catheter used for the introduction of anesthetic agents into the body.

According to some exemplary embodiments, the control circuitry 504 is configured to signal said user interface 526 to generate an alarm indication if the determined anesthesia effect indicates that the anesthesia effect is about to reach a level which is lower than a predetermined value, or has reached a level which is lower than the predetermined value.

According to some exemplary embodiments, the control circuitry 504 is configured to signal said user interface 526 to generate an alarm indication if the determined anesthesia effect indicates that the anesthesia effect is about to reach a level which is higher than a predetermined value, or has reached a level which is higher than the predetermined value.

According to some exemplary embodiments, the control circuitry 504 is configured to signal said user interface 526 to generate an alarm indication if the determined anesthesia effect indicates a unilateral anesthesia, for example a unilateral epidural anesthesia.

According to some exemplary embodiments, the control circuitry 504 is configured to signal said user interface 526 to generate an alarm indication if the level of administered anesthesia agent has reached an upper dose limit.

According to some exemplary embodiments, the system 502 comprises at least one communication circuitry, for example communication circuitry 528. In some embodiments, the communication circuitry 528 is configured to transmit and/or to receive signals from at least one device, for example a computer, located in the same room as the system 502. Alternatively or additionally, the communication circuitry 528 is configured to transmit and/or to receive signals from at least one remote device 530, for example a remote computer, a remote storage device, a cloud memory, a remote medical device and/or a server. In some embodiments, the remote device is a device located outside the room in which the system 502 is located.

According to some exemplary embodiments, the at least one communication circuitry communicates the at least one remote device 530 or with any device, for example a medical device using at least one standard protocol. In some embodiments, the at least one standard protocol comprises CEN ISO/IEEE 11073, and/or TCPIP or other standard or proprietary communication protocols. In some embodiments, communication was performed using at least one communication profile for example, Device Enterprise Communication (DEC), Alarm Communication Management (ACM), DEC Subscribe to Patient Data (SPD), and Point-of-care infusion verification (PIV) profile or other standard or proprietary profiles.

According to some exemplary embodiments, the remote device 530 stores at least one of a software program, a lookup table and an algorithm, for determining the anesthesia effect and/or the anesthesia effect distribution. In some embodiments, the control circuitry 504 transmits signals received from at least one sensing electrode, for example signals received from sensing electrodes 506 and 508, to the remote device 530 using the communication circuitry 528. In some embodiments, the control circuitry 504 receives one or more indications regarding the anesthesia effect from the remote device 530, optionally via the communication circuitry 528. In some embodiments, the remote device 530 is optionally used for at least one of, storing the signals received from the at least one sensing electrode, processing the signals received from the at least one sensing electrode, and determining the anesthesia effect and/or the anesthesia effect distribution. Optionally, the remote device classifies patients based on the results of the signal analysis, and optionally generates clusters of patients based on the signal analysis results. Optionally, the remote device uses at least one machine learning algorithm for the processing and/or analysis of the received signal.

According to some exemplary embodiments, the remote device 530 performs processing and/or analysis of a signal received from the at least one sensing electrode for example, the remote device determines a relation between a stimulation signal delivered to a tissue and the received signal, identifies different patterns in the received signal, filters and/or modulates the signal. Optionally, the remote device uses indications and data from other external databases when processing and/or analyzing the signal.

According to some exemplary embodiments, the communication circuitry 528 transmits and/or receives wireless signals from a device, for example the remote device 530.

According to some exemplary embodiments, one or more indications of at least one of, the effect of a specific anesthesia, a pharmacokinetic profile of a specific anesthesia, a pharmacodynamics profile of a specific anesthesia is stored in the remote device 530 and/or in the memory 510. Optionally, the one or more indications are stored as a database. In some embodiments, the database links at least one of, a clinical condition of a subject, a medical treatment, a medical procedure, a treatment protocol, an anesthesia dose, and an anesthesia administration rate, with the one or more indications. In some embodiments, the database stores information derived from patients receiving anesthesia that are optionally monitored using the system described herein, for example system 502. In some embodiments, the database includes information regarding the anesthesia each patient received, stimulation parameters used by the system for each patient, anesthesia effect distribution and/or other information regarding the anesthesia effect in each patient, for example as detected by the system, modifications of the anesthesia and/or modification of stimulations as performed or suggested by the system for each patient. In some embodiments, the database is used for at least one of, recording anesthesia parameter values, optimization of one or more algorithms for future patients, research regarding an effect, dosage, and/or type of anesthetic agents, when applied for regional or local anesthesia. Optionally, the remote device 530 comprises a user interface, for example, a display, configured to display the one or more indications and/or any other information received from the system to a user of the system.

According to some exemplary embodiments, the system 502 is electrically connected to an external power source. Alternatively, the system comprises at least one internal power source, for example at least one rechargeable or replaceable battery. In some embodiments, the power source complies with EN 60601-1-2:2020, IEC 61010-1:2017 and/or IEC 60601-1-8:2006/AMD2:2020 or other relevant standards.

According to some exemplary embodiments, the memory 510 comprises at least one algorithm that is used by the control circuitry to determine the current anesthesia effect, for example a depth of the anesthesia and/or a distribution of the anesthesia effect, and a relation between the current anesthesia effect and a planned, for example a desired anesthesia effect. In some embodiments, the control circuitry 504 uses the at least one algorithm after processing of the signals received from the one or more sensors, for example to remove noise.

According to some exemplary embodiments, the control circuitry 504, optionally using at least one algorithm stored in the memory 510 collects signals from the one or more sensors during a time period of at least one hour, for example at least one day or any intermediate, shorter or longer time period, and optionally generates a pharmacodynamics profile for the specific subject. In some embodiments, the control circuitry 504 collects signals before, during and/or following anesthetic administration. In some embodiments, the control circuitry generates a pharmacodynamics profile of the subject using information regarding the anesthesia administration, for example rate of administration and overall anesthesia administered to the subject.

According to some exemplary embodiments, based on the generated pharmacodynamic profile of the subject, it is possible to estimate the amount of anesthesia and/or an administration rate of anesthesia needed for different medical procedures.

According to some exemplary embodiments, the system 502 is configured to continuously monitor a neuraxial anesthesia effect by repeating stimulations through the one or more stimulating electrodes 516 and sensing the tissue response to the stimulations using the at least one sensing electrode. In some embodiments, the system 502 monitors the anesthesia effect, for example anesthesia height and/or anesthesia depth as a function of dosage of the anesthetic agents.

According to some exemplary embodiments, the system 502 is configured to process the data received from the electrodes and/or from the patient, optionally together with data stored in the memory of the system. In some embodiments, the system 502 generates an anesthesia progression trend for the progression of the anesthesia in a specific subject, based on the processed data. Optionally, the system 502 generates an anesthesia profile for the specific subject which includes information regarding the body response of the subject to one or more anesthetic agents, based on the processed data. Optionally, the system 502 is configured to generate predictions for a specific subject or a group of subjects based on the processed data.

According to some exemplary embodiments, the system 502 is configured to use the processed data, during the anesthesia process optionally in a closed-loop process, for automatically changing or suggesting to change at least one of, one or more stimulation parameters, a location of one or more stimulation electrode on a subject body, a location of one or more detecting electrodes, and at least one parameter of the anesthesia provided to the subject. In some embodiments, the one or more stimulation parameters comprise at least one of, stimulation intensity, stimulation duration, stimulation frequency, stimulation location and number of stimulation locations. In some embodiments, the at least one anesthesia parameter comprises at least one of, type of anesthesia agent, dosage, anesthesia delivery locations.

According to some exemplary embodiments, the at least one stimulating electrode and/or the at least one sensing electrode of the system 502 communicate wirelessly with the control circuitry 504, optionally via the communication circuitry 528. Optionally, the at least one stimulating electrode and the at least one sensing electrode communicate with each other, for example wirelessly. In some embodiments, each of the at least one stimulating electrode and/or the at least one sensing electrode include a power source and a wireless communication circuitry.

According to some exemplary embodiments, any wireless communication between the system 502 and a remote device, for example 530, and/or with wireless electrodes, for example wireless stimulation electrode or wireless sensing electrode, is performed using Bluetooth, Wi-Fi, infra-red or any other type of wireless communication.

According to some exemplary embodiments, when using EMG electrodes as sensing electrodes, the stimulation signal, for example electric field generated by the system has a waveform or polarity which is Biphasic, Inverse, Normal and/or any combination thereof. In some embodiments, the electric field is delivered as a train of pulses, for example as a train of 2-30 pulses, for example a train of 10-20 pulses or any intermediate, smaller or larger number of pulses per train. In some embodiments, the stimulation signal is delivered in a train rate of 1-1000 trains/second.

In some embodiments, a duration of each pulse is in a range of 100-2000 microseconds, for example 700-1200 microseconds, 800-1000 microseconds, or any intermediate, smaller or larger range of values. In some embodiments, the stimulation intensity, for example an intensity of the electric field is up to 10 mA, for example up to 15 mA, up to 8 mA or any intermediate, smaller or larger value. In some embodiments, the stimulation is delivered with a stimulation rate in a range of a 0.1-10 Hz, for example 0.1-3 Hz, 1-5 Hz, 3-10 Hz or any intermediate, smaller or larger range of values.

According to some exemplary embodiments, when measuring EMG signals, the filter 511 filters the received signal to generate signals in a range between 10-4000 Hz, and in a range between 20-200 Hz, optionally using a Notch filter. In some embodiments, the Notch filter filters the received signal within 50 Hz or 60 Hz.

According to some exemplary embodiments, when measuring EMG signals, the stimulation electrodes are positioned at dorsal locations, on the right/left side of the back, up to about 5 cm lateral to the midline. Each stimulation location comprises 2 electrodes. In some embodiments, the 2 electrodes for each location may be located horizontally to each other, to enable stimulation of same dermatome and minimizing the possibility of collecting potentials elicited at adjacent dermatomes.

Alternatively or additionally, the stimulating electrodes are located at spinal levels (the numbers of vertebrae are reference locations): T6-L4, for example when an epidural injection is at about L1/2.

According to some exemplary embodiments, when measuring neural activity, the at least one sensing electrode is positioned at cortical and/or sub-cortical location. Alternatively or additionally, when measuring EMG, the at least one EMG electrode is positioned on a face of a subject.

According to some exemplary embodiments, the control circuitry 504 is configured to measure a subject body response by analyzing at least one signal received from at least one sensing electrode using at least one algorithm, for example a machine learning algorithm stored in the memory 510. In some embodiments, the machine learning algorithm, for example an algorithmic classifier, is configured to categorize portions of the at least one received signal into at least two groups comprising a first group of signal portions indicating a positive transmission of sensory information, and a second group of signals indicating a block in transmission of sensory information. In some embodiments, the control circuitry 504 determines the anesthesia effect based or using the analysis results.

According to some exemplary embodiments, the system 502, for example the control circuitry 504 is configured to process the signal received for the at least one sensing electrode, for example by filtering the received signal using a band pass filter and/or a notch filter, optionally stored in the memory of the system, for example memory 510. Optionally, the control circuitry 504 applies at least one signal amplifier stored in the memory on the received signal, for example a low noise amplifier (LNA), and/or a power amplifier (PA). In some embodiments, the LNA comprises at least one of, Analogy Designed LNA, Digitally Designed LNA, and Adaptive LNA. Optionally, the control circuitry 504 applies at least one signal modulation technique on the received signal, for example Pulse Width Modulation, Pulse Duration Modulation, Frequency Modulation, and Phase Modulation. Reference is now made to FIG. 5B depicting a system for monitoring anesthesia effect, according to some exemplary embodiments of the invention.

According to some exemplary embodiments, the system, for example system 502 or system 550, is used by and is in communication with a user, for example a patient, that undergoes medical procedures which require continuous anesthetics injection into the epidural space. In some embodiments, this segment of users views parameters by a designated Patient Control Device (PCD). Alternatively or additionally, a user of the system comprises at least one of an anesthesiologist, professional and/or clinicians. In some embodiments, this segment of users controls the system, and/or view parameters by a designated Patient Control Device (PCD) and/or Multi-patients Remote User Interface (MRU), for example as shown in FIG. 5D. Alternatively or additionally, a user of the system comprises a system technician. In some embodiments, this segment of users includes all the relevant professionals which are qualified to operate the system, provide technical solutions and backups. These users will have an access to all of the components.

According to some exemplary embodiments, the system measures a tissue response to stimulation, for example to characterize a clinical state of a subject and/or to adjust one or more parameters of a treatment to a clinical state of the subject.

According to some exemplary embodiments, the system is used for determining appropriate epidural catheter insertion. In some embodiments, an anesthesiologist inserts the epidural catheter into the epidural space and initially determines the amount and rate of the drug flow. In some embodiments, within few minutes the system will identify reduction in sensorial activity which indicates a normal epidural anesthesia. In some embodiments, this indication will enable the anesthesiologist to assume a successful catheter insertion and expect normal anesthesia expansion.

According to some exemplary embodiments, the system is used for identifying misplaced epidural catheter insertion. In some embodiments, the Anesthesiologist inserts the epidural catheter and initially determines the amount and rate of the drug flow. In some embodiments, within few minutes the system identifies that a change in sensorial activity is not according to a predetermined reduction or an expected reduction, which indicates a potential problem in the epidural anesthesia. In some embodiments, this indication will enable the Anesthesiologist to assume a misplaced catheter insertion and optionally to repeat the catheter insertion procedure.

According to some exemplary embodiments, the system is used to monitor appropriate dose administration. In some embodiments, few minutes, for example 15-30 minutes after commencing drug administration the system indicates a current anesthesia effect, that can optionally be used to determine a gap between the current anesthesia effect and a desired anesthesia effect and/or optimal anesthesia conditions. Additionally, the patient does not report excessive pain and no alerts or notifications are displayed on the Local User Interface (LUI), for example as shown in FIG. 5C and optionally displayed on the Multi-Patients Remote User Interface (MRU), for example as shown in FIG. 5D. In some embodiments, the system keeps monitoring the depth and axial height of the anesthesia and suggests the anesthesiologist if and how much to change the administered dose in order to keep the anesthesia at an optimal level.

According to some exemplary embodiments, the system is used for detecting that an anesthetized area is too small. In some embodiments, upon normal delivery progress, the anesthesiologist may be required to anesthetize a wider area of the patient body in order to ensure the normal progress of the delivery. The system suggests appropriate amount of drug (bolus) to be manually appended by the anesthesiologist (Or to be automatically appended by the delivery system after confirmation of the Anesthesiologist) according to the situation.

According to some exemplary embodiments, the system is used for detecting that an anesthesia depth is too low. In some embodiments, while monitoring anesthesia depth over time the system detects that although the anesthesia covers a desirable area, its depth is too low which in turn may cause pain to the patient. In some embodiments, the system is configured to deliver an alert signal, for example to display a warning indication, and optionally suggests the anesthesiologist to increase the drug dose concentration or dosage (i.e., from 1% to 2%) in order to gain/regain optimal anesthesia depth.

According to some exemplary embodiments, the system is used to detect that an anesthetized are is too large, which may lead to an overdose condition. In some embodiments, while monitoring an anesthesia effect following administration of an anesthetic drug, the system detects that the anesthetized area is too large, for example larger than a predetermined area. In some embodiments, when the anesthetized area is tool large, undesirable side effects such as motor weakness or hypotension can occur, which in turn may cause the patient not to be able to take part in a medical procedure, for example a delivery of a child. In some embodiments, the system is configured to display an alarm indication suggesting the anesthesiologist to take immediate supporting actions to mitigate the emerging symptoms and/or reduce the drug flow in order to reduce the area under anesthesia.

According to some exemplary embodiments, the system is used in order to detect that a depth of anesthesia is too deep. In some embodiments, at any time after commencing drug administration the system detects that the anesthetized level (depth) is too high even though the anesthetized area is as desired and as defined by the anesthesiologist. This situation may cause undesirable side effects such as Motor Block. The system will display a warning indication suggesting the Anesthesiologist to take immediate supporting actions to mitigate the emerging symptoms and reduce the drug dosage or concentration (i.e., from 2% to 1%) in order to gain/regain optimal anesthesia level.

According to some exemplary embodiments, the system is used for detecting Hemiparesis, an anesthesia of only one side of the body. In some embodiments, 0.5-30 minutes following commencing drug administration onward, the system detects that only one side of the body is anesthetized, optionally caused by a reduced effect of the anesthesia which in turn may cause pain. In some embodiments, the system detects Hemiparesis based on measurements and/or stimulations performed in both sides of the body, for example on both sides of the spinal cord. In some embodiments, the system is configured to provide an alert signal, for example to display a warning and optionally to suggest the anesthesiologist to increase the drug dosage and/or drug flow (according to the situation), to optionally increase the anesthetized area or deepen the level of anesthesia or to change the location of the epidural catheter.

According to some exemplary embodiments, the system is used for detecting early recovery from anesthesia, for example as described above with respect to detecting a lower dose of anesthesia.

According to some exemplary embodiments, the system is used in order to preplan an anesthesia protocol for different emergency conditions. In some embodiments, during a neuraxial anesthesia procedure, the patient may enter into an emergency condition forcing an emergency clinical intervention such as Caesarean Section or instrumented delivery (vacuumed). In some embodiments, the system suggests an appropriate additional dosage (top-up) of anesthesia which is required as preparation for the upcoming procedure. In some embodiments, the system suggests how to modify an existing anesthesia protocol to make it suitable for a new medical procedure, based the monitoring of the anesthesia effect and/or based on a generated pharmacodynamics profile for the specific patient. Optionally, the suggested dosage may be different for each type of emergency.

According to some exemplary embodiments, a clinician remotely views specific patient's information in a similar way as it is displayed on the Local User Interface (LUI).

According to some exemplary embodiments, the system is used to monitor an anesthesia effect or a stat of anesthesia in a plurality of patient, for example using Multi-Patient Remote User Interface (MRU) shown in FIG. 5D. In some embodiments, the system provides a dashboard view on the Multi-Patient Remote User Interface (MRU). In some embodiments, the dashboard view is configured to allow visualizing at a glance if there is a situation requiring Anesthesiologist intervention.

According to some exemplary embodiments, the system is used to collect, store and/or analyze data. In some embodiments, the system stores anesthesia-related data for a specific patient, for example to allow retrieval of the data in the future. Alternatively, or additionally, the system is used to collect, and analyze data from plurality of subject in order to generate a database, and/or to generate improved and/or personalized anesthesia administration protocols.

According to some exemplary embodiments, an anesthesia monitoring system, for example system 502 or system 550 is configured to produce topical electric stimulations. In some embodiments, the electric stimulations are provided with parameter values, for example intensity, frequency and pulse width, that are higher than a sensation threshold value of a non-anesthetized body area. In some embodiments, in case the area is anesthetized this threshold is assumed to be higher than the delivered stimulation, therefore there will be no signal delivery by the nerve from the location of the stimulation up to the brain. Alternatively, in case the stimulated area is non-anesthetized, the signal is delivered to the brain which in turn initiates a sensation signal and/or pain-associated physiologic indications.

According to some exemplary embodiments, the system is configured to detect the presence or the absence of neural signals, for example either D-SSEP and/or EMG signals. In some embodiments, a controller 552 (SCU) that is wired to an array of stimulating electrodes 554 (SEA) activates the electrodes at various axial heights according to a pre-planned protocol. In some embodiments, the controller 552 is part of the control circuitry 504 shown in FIG. 5A. In some embodiments, the protocol is a set of operational parameters such as neural signal type (D-SSEP/EMG), sequence of activation, signal shape, signal duration, signal intensity, signal frequency etc.

According to some exemplary embodiments, the controller 552 also controls one or more sensing electrode 556 (SSE) which are operable to collect the signal as it is received at a subcortical location (rear part of the head, nape, back, cervical region and/or shoulder) and/or cortical locations of the patient. In some embodiments, the collected signal is analyzed by a processing unit 558 (SPU). In some embodiments, the SPU 558 is part of the control circuitry 504 shown in FIG. 5A. In some embodiments, the process, in turn, utilizes a detection algorithm, stored in a memory of the system, containing model coefficients over the stream of data. In some embodiments, the algorithm detects the presence, the deviation and/or the absence of the signal from the sensing location/s in correlation with the stimulating signal/s, which allows optionally estimation of an implication of the anesthetic drug.

According to some exemplary embodiments, the SPU 558 is configured to generate the pharmacodynamic profile described with regard to the control circuitry 504 shown in FIG. 5A. In some embodiments, the SPU 558 generates the pharmacodynamics profile of one or more anesthetic compounds used for an anesthesia in a subject, based on a determined anesthesia effect and/or one or more subject-related indications stored in a memory, for example memory 510 shown in FIG. 5A. In some embodiments, the subject-related indications comprise one or more indications related to a clinical state of the subject, age, gender, BMI, medical history, and/or drug regime.

According to some embodiments, the stream of data is collected from one or more patients to generate the model coefficients. In some embodiments, the model coefficients are generated using offline data processing, using data from one or more patients. Optionally or alternatively, the data processing is performed before using the system 550 on a new patient.

In some embodiments, the stream of data is collected from one or more patients and stored on an external server for example, a cloud or a remote server, for generating the model coefficients. In some embodiments, the model coefficients are extracted from the external server and use as an input for the processing unit 558 (SPU).

According to some embodiments, the stream of data is collected from one or more patients to generate the pharmacodynamics profile. In some embodiments, the pharmacodynamics profile is generated using offline data processing, using data from one or more patients. Optionally or alternatively, the data processing is performed before using the system 550 on a new patient.

In some embodiments, the stream of data is collected from one or more patients and stored on an external server for example, a cloud or a remote server, for generating the pharmacodynamics profile.

According to some exemplary embodiments, at any given time during neuraxial anesthesia the system may automatically or semi-automatically, for example when there is a need to receive a user approval, change the operation parameters of the signal stimulation. In some embodiments, the parameters under such automatic or semi-automatic control comprise at least one of signal intensity, signal duration, stimulation location (electrode) and signal spreading. In some embodiments, a decision to change the operation parameters are based on normal or abnormal spreading of the anesthesia throughout the patient body. In some embodiments, some situations may call for lowering the signal spreading to case patient's fatigue, other situation may call for lowering the signal intensity in case the signal is easily detected etc.

According to some exemplary embodiments, at any time, the anesthesiologist is altering the drug amount or the dose concentration or the dosage he will be prompt to insert the information of the change (i.e. add 2 ml of drug, or change the concentration from 1% to 2%), via a user interface of the system, for example via a clinical input screen.

According to some exemplary embodiments, at some points of the clinical procedure the patient provides human feedback of his/her experience i.e., in pain, feels no pain, feels cold or feels pin prick. In some embodiments, the feedback of the patient is provided via the user interface of the system, for example via a patient feedback interface optionally comprising a screen.

According to some exemplary embodiments, the model coefficients are obtained by training sets of signal data that are collected offline at the development phase of the algorithm. In some embodiments, the training sets are run through a Machine Learning set of algorithms that derives the model coefficients required for the on-the-spot signal detection.

According to some exemplary embodiments, the system 502 is configured to generate one or more types of alert signals, for example a first type of alert signals indicates insufficient distribution of anesthesia, a different type of an alert signal indicates anesthesia distribution that exceeds a predetermined anesthesia height, a different type of an alert signal indicates asymmetric distribution of anesthesia. In some embodiments, the system 502 delivers the alert signal via the user interface 526 and/or by transmitting the generated alert signal to the remote device 530 using the communication circuitry 528.

### Exemplary User Interface

Reference is now made to FIG. 5C depicting a single patient user interface, for example a local user interface (LUI), according to some exemplary embodiments of the invention.

According to some exemplary embodiments, a user interface, for example a LUI 570 delivers one or more indications, for example one or more visual indications, to a user of the system. In some embodiments, a control circuitry, for example control circuitry 504 signals the user interface to generate the one or more indications. Alternatively, a local web server, for example local web server 557 signals the user interface, for example LUI 570 to generate the one or more indications. In some embodiments, the one or more visual indications comprise indications related to axial distribution of anesthesia, for example anesthesia height. In some embodiments, the one or more indication comprises indications regarding a current anesthesia height and/or a desired anesthesia height. In some embodiments, the one or more visual indications comprise indications regarding changes in anesthesia effect over time, optionally compared to a desired, for example a target anesthesia effect. In some embodiments, the one or more indications comprise indications regarding at least one of, stimulation parameters, impedance, electric density per electrode, a stimulation location by at least one stimulating electrode and/or indications regarding a sensing location by at least one sensing electrode. Optionally, the LUI is configured to provide the one or more indications per a single patient, for example during a medical procedure. In some embodiments, the one or more indications comprise an alert signal, indicating for example when the determined anesthesia effect is not within a desired range of anesthesia effect values.

Reference is now made to FIG. 5D depicting a multi-patients user interface, for example a Multi-patients Remote User Interface (MRU), according to some exemplary embodiments of the invention.

According to some exemplary embodiments, the MRU 578 delivers one or more indications, for example one or more visual indications, to a user of the system. In some embodiments, the one or more indications comprise indications regarding a status of anesthesia and/or an anesthesia effect in two or more patients, optionally simultaneously, from a single point of view. In some embodiments, anesthesia effect, for example anesthesia height and/or anesthesia depth per each patient is presented to the user of the system, for example to an expert or a caregiver monitoring the anesthesia effect in the patients.

In some embodiments, the one or more indications regarding the anesthesia effect and/or status per patient in the single user interface and/or the multi-patient user interface, is updated every time period which is shorter than 1 minute, for example every 30 seconds, every 10 seconds, every 5 seconds, every 1 second, every 0.5 second or any intermediate, smaller or larger value.

According to some exemplary embodiments, when delivering indications regarding a plurality of subjects, the MRU 578 generates and delivers alarm indications according to predefined severity scenarios, stored in the system memory.

### Exemplary System and Algorithm

According to some exemplary embodiments, a system, for example system 502 shown in FIG. 5A, or the system shown in FIG. 5B, or part of a system, includes a memory with at least one algorithm and/or a set of instructions stored in the memory. In some embodiments, the algorithm and/or the instructions are used during the operations and activities of the system, or a part of the system. In some embodiments, the system is configured to automatically deliver a stimulation to an anesthetized subject, and to measure a response of the subject to the delivered stimulation. In some embodiments, the system determines an effect of the delivered anesthesia based on at least one of, anesthesia parameters, stimulation parameters and/or the measured response of the subject. Additionally, the system determines an effect of the delivered anesthesia by determining a relation between the at least one of, anesthesia parameters, stimulation parameters and/or the measured response of the subject, and one or more indications stored in the memory. In some embodiments, the one or more indications comprise at least one of, previously used stimulation parameters, previously measured response of the subject to stimulation, previously used anesthesia parameters, at least one anesthesia protocol, a pharmacodynamics profile and/or at least one prediction.

According to some exemplary embodiments, a detected response of the subject to stimulation is compared to a predicted response of the subject, for example in order to determine the anesthesia effect on the subject.

According to some exemplary embodiments, the system is configured to determine an anesthesia depth, for example whether a specific tissue or tissue region is under a deep anesthesia effect and/or whether a tissue or a region is under a light anesthesia effect, thereby classifying the effect of the anesthesia on the tissue or region, by determining a relation between values of the stimulation parameters used for stimulation, for example frequency, intensity and duration, and the measured response of the subject. In some embodiments, the algorithm of the system receives the responses of the subject to stimulation, for example measured responses or input from the subject, and is configured to classify the responses as a “detected response”, where the stimulation was delivered to a region that is not anesthetized, or as a “non-detected response”, where the stimulation is provided to an anesthetized region. Optionally, the algorithm is configured to generate 3 or more classifications according to different threshold levels of the measured response, for classifying the response of the tissue.

According to some exemplary embodiments, the algorithm and/or the set of instructions determine at least one parameter of stimulation sequence delivered to the subject body, for example to determine one or more parameters of the stimulation, and optionally which electrode of a plurality of stimulation electrodes to use and/or when to use the electrode for delivery of the stimulation. In some embodiments, the algorithm and/or the set of instructions include information whether to use a single stimulating electrode, a specific set of two or more stimulation electrodes or a set of stimulation electrodes and/or whether to use all the stimulation electrodes. In some embodiments, the algorithm and/or the set of instructions determine the at least one stimulation parameter according to a state, for example a clinical state and/or history of a subject. In some embodiments, the system, for example a control circuitry of the system, generates and delivers the stimulation using the algorithm and/or the set of instructions stored in the memory.

According to some exemplary embodiments, the system uses the algorithm in order to generate a pharmacodynamics profile of the subject, for example based on detected responses of the subject to a plurality of delivered stimulations. In some embodiments, the algorithm is used to update the pharmacodynamics profile based on additional detected responses and/or based on additional data received or measured form the subject.

According to some exemplary embodiments, the system, optionally using the algorithm and/or the set of instructions, is configured to generate a prediction and/or to generate an alert indication before the anesthesia effect drops below a predetermined value or a predetermined threshold, and/or before the anesthesia effect is higher than a predetermined value or a predetermined threshold, which may lead to unwanted side effect, according to the pharmacodynamic profile.

According to some exemplary embodiments, the system, optionally using the algorithm and/or the set of instructions, is configured to provide suggestions to a physician or an expert controlling anesthesia, regarding a dose or to suggest an increase of dose, of at least one anesthetic drug that needs to be provided to a subject in order to reach a desired anesthesia effect. For example, the system is configured to provide suggestions to the physician or expert regarding a dose or to suggest an increase in a dose, of at least one anesthetic drug delivered to a female subject undergoing childbirth when there is a need to move the female subject to a surgery room, and therefore a deeper anesthesia effect is needed.

Additionally or alternatively, the system, optionally using the algorithm and/or the set of instructions, is configured to provide suggestions to a physician or an expert controlling anesthesia with information regarding the time after stopping the delivery of anesthesia that is needed in order to reduce the anesthesia effect to minimum or below a predetermined value.

### Exemplary Detailed Process for Anesthesia Delivery

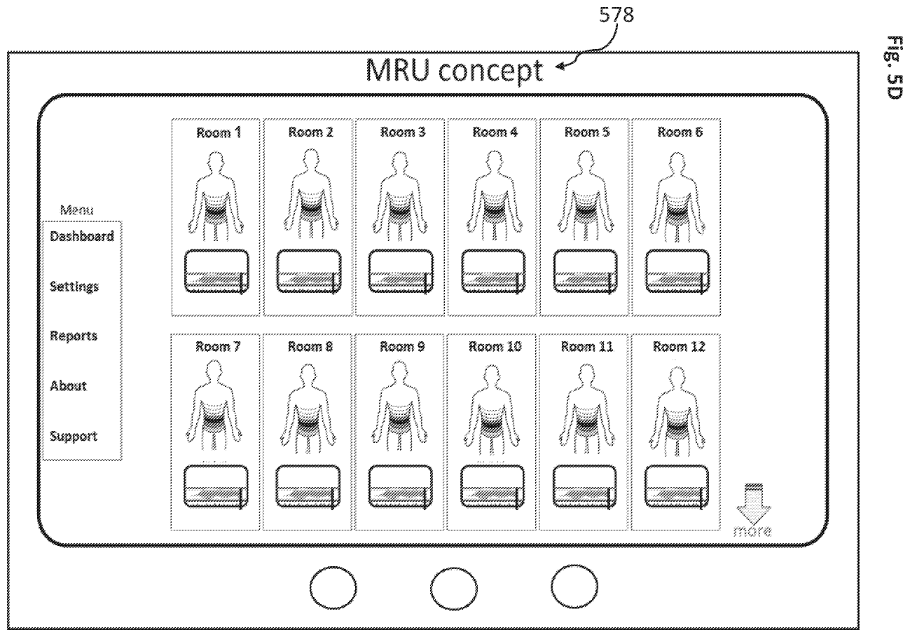

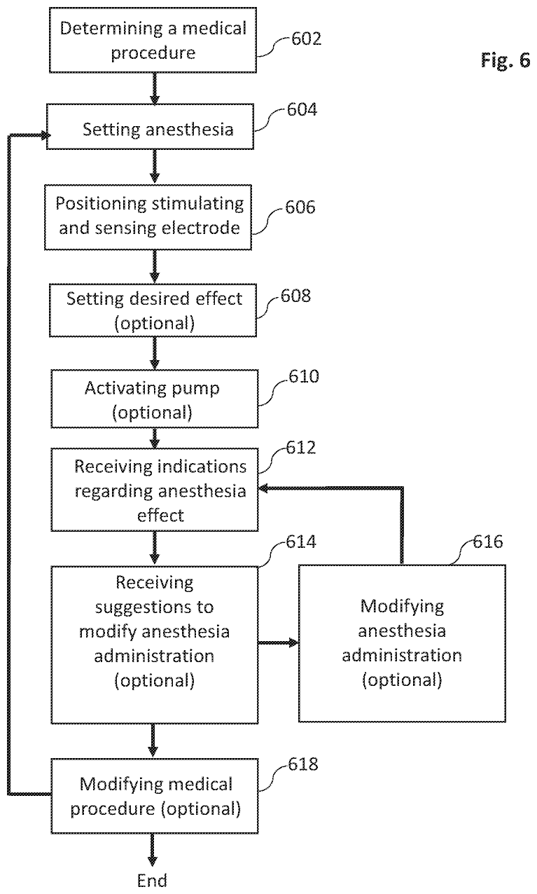

According to some exemplary embodiments, at least one parameter related to anesthesia effect, for example anesthesia height or anesthesia depth, is determined prior to and/or during a medical procedure, for example a treatment. In some embodiments, the anesthesia effect is optionally determined in order, for example, to make sure that an effect of anesthesia on the tissue is according to a predetermined, desired, target effect. In some embodiments, an anesthesia target site is a location in the body, for example a body region, where an effect of anesthesia is required, optionally in order to initiate and/or during a medical procedure. Reference is now made to FIG. 6, describing a process for determining an effect of anesthesia by a user of the system, according to some exemplary embodiments of the invention.

According to some exemplary embodiments, a medical procedure, for example a treatment, is determined at block 602. In some embodiments, a medical procedure for example a childbirth, is planned at block 602. In some embodiments, the medical procedure plan comprises one or more of, determining if anesthesia is required, determining a desired effect of anesthesia on a target site, determining type and composition of anesthesia, determining anesthesia dose, and determining anesthesia infusion site.

According to some exemplary embodiments, at least one parameter of the anesthesia is set at block 604. In some embodiments, the at least one parameter comprises type of anesthesia, anesthesia composition, anesthesia dose, anesthesia administration rate and/or the anesthesia infusion site. In some embodiments, a user of the system inserts the at least one anesthesia parameter into a memory of the system, for example memory 510 shown in FIG. 5A via the user interface 526. Optionally, the system provides recommended settings of the at least one anesthesia parameter. Optionally, the recommended settings of the at least one anesthesia are provided by the system according to at least one of, the determined medical procedure, clinical state of the subject, medical history of the subject, and personal characteristics of the subject, for example age and/or gender.

According to some exemplary embodiments, at least one stimulating electrode and at least one sensor, for example a sensing electrode are positioned at block 606. In some embodiments, the at least one stimulating electrode, for example stimulating electrodes 516 shown in FIG. 5A, and the at least one sensing electrode, for example sensing electrodes 506, 507, 508 and 509 are positioned according to the medical procedure plan determined at block 602. Alternatively or additionally, the at least one sensing electrode and/or the at least one stimulating electrode is positioned according to a location of the target site and/or according to the location of neurons or at least one neural network innervating the target site.

According to some exemplary embodiments, the at least one stimulating electrode and/or the at least one sensor is optionally positioned relative to a body of the subject, for example on a body of the subject or close to the subject body. Alternatively, at least one stimulating source and/or the at least one sensor is positioned at a distance from the subject body, for example at a distance in a range of 10 cm to 3 meters, at a distance in a range of up to 15 cm form the subject body, at a distance in a range of 20 cm to 2 meters from the subject body or in any intermediate, shorter or larger distance from the subject body.

According to some exemplary embodiments, prior to positioning, a number of stimulating electrodes and/or a number of sensors, for example sensing electrodes is selected. In some embodiments, the number of stimulating electrodes and/or the number of sensors is selected according to the medical procedure plan and/or an expected anesthesia effect distribution.

According to some exemplary embodiments, the system for anesthesia effect monitoring, for example system 502 shown in FIG. 5A, delivers instructions to a user with regard to at least one of, the treatment plan, number of stimulating electrodes and/or sensors and a recommended position for the stimulating electrodes and/or sensors.

According to some exemplary embodiments, a desired anesthesia effect is optionally set, at block 608. In some embodiments, a desired anesthesia effect on at least one target site, for example a target site surrounding the injection site, is optionally set. In some embodiments, the desired anesthesia effect, is optionally set at block 608. Alternatively or additionally, the desired anesthesia effect on a target site during a medical procedure, for example as shown in FIGS. 4A-4C is optionally set at block 608.

According to some exemplary embodiments, at least one actuator, for example a pump 522, shown in FIG. 5A, is optionally activated at block 610. In some embodiments, the at least one actuator is activated according to indications of settings stored in the memory of the system, for example memory 510 shown in FIG. 5A. In some embodiments, the at least one actuator is activated when receiving an input signal from a user of the system, optionally using the user interface 526.

According to some exemplary embodiments, the user, for example a physician, a patient, a nurse, a supervisor, or a technician, receives indications regarding the anesthesia effect, at block 612. In some embodiments, the system delivers the indications to the user using the user interface 526. In some embodiments, the deceived indications include information regarding a current distribution of the anesthesia effect, optionally with respect to the target site. Alternatively or additionally, the received indications include information regarding an expected anesthesia effect after one or more selected time periods. Alternatively or additionally, the received indications include information regarding the level of anesthesia effect at one or more measurements sites in the body and/or at the target site. In some embodiments, the user receives indications regarding the anesthesia effect at block 612 from the system. Alternatively, the user receives indications regarding the anesthesia effect at block 612 from a device in communication with the system, for example a remote device, for example a remote server, a remote computer, a remote cloud storage, that is in communication with the system, for example as described in FIG. 5A. Alternatively, the system generates the indication regarding the anesthesia effect as part of an internal feedback process. In some embodiments, the indication is stored in a memory of the system.

According to some exemplary embodiments, the system generates an indication with information whether or not a specific body region is under anesthesia effect and/or the level of anesthesia effect in a specific body region, based for example, on a determined relation between the stimulation, for example stimulation intensity, delivered to the subject by the system and a signal received by at least one sensing electrode. In some embodiments, the system determines the relation using one or more algorithms, for example algorithmic classifiers, optionally applied on the received signal and/or any other data stored in a memory of the system, for example data stored in a remote device, or a database, in communication with the system.

According to some exemplary embodiments, the indications received by the user at block 612 comprise at least one alert signal. In some embodiments, the system generates the at least one alert signal when a measured anesthesia effect is not according to a a desired effect, for example as defined at block 608. Alternatively or additionally, the at least one alert signal indicates a change in a clinical state of a subject being monitored, for example an undesired change in maternal vital signs or fetal heart rate. In some embodiments, the at least one signal is generated using the user interface, for example user interface 526 shown in FIG. 5A, or for example by a remote device receiving a signal from the system. Optionally, the at least one alarm signal is transmitted to the remote device, for example to an external medical care system using ACM (Alarm Communication Management) protocol.

According to some exemplary embodiments, the user optionally receives suggestions to modify anesthesia administration at block 614. In some embodiments, if the anesthesia effect is lower than a predetermined value or lower than expected, the user receives at least one suggestion to increase anesthesia dose, increase anesthesia administration rate, and/or modify anesthesia composition or ratio between bioactive compounds in the anesthesia. In some embodiments, if the anesthesia effect is higher than a predetermined value or higher than expected, the user receives at least one suggestion to reduce administration dose, reduce anesthesia administration rate and/or to stop anesthesia administration. In some embodiments, the user optionally receives the at least one suggestion to modify anesthesia administration at block 614, from the system, for example using the user interface. Alternatively, the user optionally receives the at least one suggestion from a device in communication with the system, for example a remote device. Alternatively, the system automatically modifies anesthesia administration or parameters thereof, for example based on the indication generated at block 612.

According to some exemplary embodiments, the system may issue a human detectable signal, for example an alarm. The alarm may be issued as a result of deviation between a desired anesthesia effect and a current anesthesia effect.

In some embodiments, the alarm may be transmitted to external medical care system using an ACM (Alarm Communication Management) protocol.

According to some exemplary embodiments, anesthesia administration is optionally modified at block 616. In some embodiments, the anesthesia administration is optionally modified according to the at least one suggestion received at block 614. In some embodiments, the anesthesia administration is optionally modified by reprogramming the system based on an input received from the system user.

According to some exemplary embodiments, a medical procedure is optionally modified at block 618. In some embodiments, the medical procedure is modified, for example due to complications and/or due to changes in a clinical state of the subject. In some embodiments, when a medical procedure is optionally modified, then one or more settings of the system, for example number and position of at least one stimulating electrode, one or more stimulation parameters, and/or at least one sensor are also optionally modified. Additionally or alternatively, when a medical procedure is optionally modified, then at least one parameter related to the anesthesia, for example to the anesthesia administrations and/or a desired effect of the anesthesia, for example as described at blocks 606 and 608 is optionally modified.

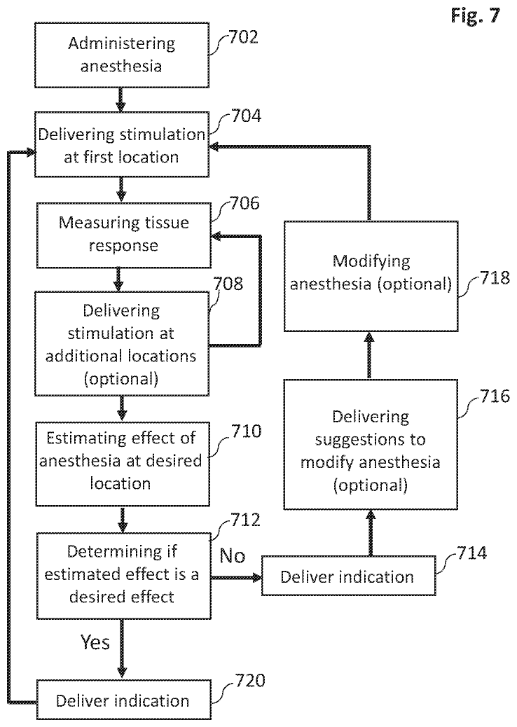

Reference is now made to FIG. 7 depicting a process performed by a system for delivery of anesthesia, according to some exemplary embodiments of the invention.

According to some exemplary embodiments, the system, for example system 502 shown in FIG. 5A, administers anesthesia at block 702. In some embodiments, the system administers the anesthesia according to one or more indications stored in a memory of the system, for example memory 510 shown in FIG. 5A. Alternatively or additionally, the system administers the anesthesia according to input received from a user, for example using the user interface 526, and/or physiological information measured form the patient.

According to some exemplary embodiments, the system delivers stimulation, for example electric stimulation optionally by a delivery of an electric field, sensory stimulation, thermal stimulation, pressure stimulation, and/or tactile stimulation at block 704. In some embodiments, the stimulation is delivered by applying vacuum, pinch, vibration, pressure, humidity, touch and/or pain on the patient. In some embodiments, the system delivers the stimulation according to parameter values stored in the memory, for example memory 510. In some embodiments, the sensory stimulation comprises pain stimulation higher than a pain threshold, or stimulation below the pain threshold. In some embodiments, the system delivers the stimulation through one or more stimulating electrodes, for example stimulating electrodes 516. In some embodiments, the one or more stimulating electrodes are optionally attached to a body of the subject. Alternatively one or more stimulating sources are positioned at a distance from the subject body, for example at a distance larger than 5 cm, for example larger than 10 cm, larger than 15 cm, larger than 20 cm from the subject body, or at a distance of up to 15 cm from the body.

In some embodiments, the one or more stimulating electrodes are attached to a back of the subject. In some embodiments, the one or more stimulating electrodes comprise a plurality of electrodes axially arranged along a longitudinal axis of the body on the back of the patient. In some embodiments, the electrodes are arranged on both sides of the spinal cord, or on a single side of the spinal cord. In some embodiments, the electrodes are arranged according to a dermatomes arrangement of the body, such that each electrode is attached and stimulates a different dermatome. Alternatively or additionally, one or more stimulating electrodes are positioned on an abdomen, chest and/or at least one limb of the patient. Alternatively or additionally, the one or more stimulating electrodes are located on a waist line of the patient. Alternatively, the one or more stimulating electrodes are randomly distributed on the patient body.

According to some exemplary embodiments, the stimulation, is delivered to the subject body at a first location. In some embodiments, the first location is a location in which at least one stimulation electrode is positioned. In some embodiments, the first location is selected based to a distance from an anesthesia infusion site and/or based on a distance of the first location from a target site.

According to some exemplary embodiments, the system delivers the stimulation at block 704 with parameter values selected to induce a response, for example a sensory response in the subject. In some embodiments, the parameter values of the stimulation are selected in order to induce a response reaction, for example a sensory response in the subject that is mediated by one or more neurons in the spinal cord and/or in the brain of the subject. Alternatively or additionally, the parameter values of the stimulation are selected in order to induce a muscle response, for example changes in muscle activity or changes in the electrical activity of the muscle. In some embodiments, the delivered stimulation comprises delivery of an electric field to the body of the subject, for example at the first location.

According to some exemplary embodiments, stimulation is delivered in a sequence or simultaneously from two or more stimulating electrodes located at different distances from an anesthesia infusion site. In some embodiments, stimulation is first delivered at a first location which is close to the anesthesia infusion site, and later at one or more additional distal locations, for example locations that are more distant from the anesthesia infusion site compared to the first location.

According to some exemplary embodiments, the system measures the tissue response, at block 706. In some embodiments, the system measures the tissue response following the stimulation delivered at block 704. In some embodiments, the tissue response is measured at a second location located at a distance from the first location to which stimulation was delivered. In some embodiments, the tissue response is measured by one or more sensors, for example one or more sensing electrodes 506 and/or 508 shown in FIG. 5A. In some embodiments, the one or more sensors are attached to the body of the subject or are located within the subject body. Alternatively, the one or more sensors, for example thermal sensors, are positioned at a distance from the subject body. In some embodiments, the one or more sensors comprise electrodes that record neural activity, for example brain activity by measuring an ERP for example SSEP, before, during and after the delivery of stimulation. In some embodiments, the recorded neural activity, for example brain activity is indicative to a tissue response to the delivered stimulation. Alternatively or additionally, the one or more sensors comprise electrodes that record muscle activity or electrical activity of a muscle, for example EMG electrode.

According to some exemplary embodiments, stimulation is optionally delivered in a sequence or simultaneously at additional locations, at block 708. In some embodiments, the stimulation is delivered from two or more stimulating electrodes, optionally arranged as an array of stimulating electrodes. In some embodiments, each of the stimulating electrodes is located at a different distance from an anesthesia infusion site, and/or at a different dermatome and/or at a different side of the spinal cord or both sides of the spinal cord. In some embodiments, a response of the tissue to the multiple stimulations is measured during and/or following the stimulations. In some embodiments, the tissue response, for example an evoked response, is measured at a single measurement site or at two or more measurement sites. In some embodiments, a tissue response is measured after each stimulation. Alternatively, the tissue response is measured after one or more selected stimulation of two or more stimulations.

According to some exemplary embodiments, an effect of anesthesia is estimated at block 710. In some embodiments, the anesthesia effect comprises anesthesia effect distribution. In some embodiments, the anesthesia effect is estimated by the system, for example by a control circuitry of the system. In some embodiments, the anesthesia effect is estimated based on signals received from the one or more sensors. In some embodiments, the anesthesia effect is estimated by processing of the received signals, and measuring the anesthesia effect using one or more algorithms and/or lookup tables stored in the memory of the system. In some embodiments, the one or more algorithms and/or lookup tables optionally describe a relation between signals received from the one or more sensors or processed signals, and anesthesia effect values, or they can be used to calculate the relation. In some embodiments, measuring or determining of the anesthesia effect is by determining a relation between signals detected from the subject and one or more stimulation parameters.

According to some exemplary embodiments, the system a relation between estimated and desired effect, at block 712. In some embodiments, the system determines if the estimated effect is a planned effect, for example by determining a relation between the estimated effect, and indications stored in the memory of the system. In some embodiments, the system determines if the estimated effect is a planned effect, for example using one or more algorithms and/or lookup tables stored in the memory of the system. In some embodiments, the one or more algorithms and/or lookup tables optionally describe a relation between estimated effect, for example values of a measured effect, and a planned effect, for example a desired effect. Alternatively, the one or more algorithms and/or look-up tables are optionally used to calculate the relation.

According to some exemplary embodiments, estimating the anesthesia effect and/or determining if an estimated effect is a desired effect is performed in a device that is in communication with the system, for example in a remote device. In some embodiments, the device receives signals from the system and estimates the anesthesia effect and/or determines if the estimated effect is a desired effect using one or more algorithm and/or lookup tables stored in the device. In some embodiments, the device uses the conclusions of the estimated anesthesia effect and/or the determining if the estimated effect is a desired effect to update one or more models or algorithms stored, and/or to generate a database. In some embodiments, the device transmits indications of the conclusions to the system. Alternatively, the device transmits indications of the conclusions to a different device, for example to a remote device. In some embodiments, the indications comprise at least one alarm indication. In some embodiments, the remote device comprises a cellular or a mobile device of an expert or a user of the system, or a medical device at the point of care or any different medical device, optionally using standard medical protocols.

According to some exemplary embodiments, if the estimated effect is not a desired effect, then an indication is delivered at block 714. Optionally, the indication is an alert signal. In some embodiments, the indication comprises a human detectable indication delivered to a user of the system, optionally by the user interface. Alternatively or additionally, an indication is transmitted to a device, for example a remote device in communication with the system.

According to some exemplary embodiments, one or more suggestion to modify the anesthesia is optionally generated and delivered at block 716. In some embodiments, the one or more suggestion is delivered by a user interface of the system. In some embodiments, the one or more suggestion comprises the one or more suggestion received at block 614 described in FIG. 6.

According to some exemplary embodiments, the system optionally automatically modifies the anesthesia at block 718. In some embodiments, if the estimated effect is not a target effect, for example a desired effect, the system optionally automatically modifies a rate of anesthesia administrations, and/or administers one or more different anesthetic agents. Alternatively, if the estimated effect is not a desired effect, for example if the estimated effect is higher than a desired effect, then the system optionally automatically stops anesthesia administration.

According to some exemplary embodiments, if the estimated anesthesia effect is a desired effect, then the system generates and delivers a human detectable indication to a user of the system. Alternatively or additionally, if the estimated anesthesia effect is a desired effect, then the system transmits an indication to a device that is in communication with the system, for example a remote device. Optionally, the remote device comprises a cellular or a mobile device.

### Exemplary Use of the System During Childbirth

According to some exemplary embodiments, the system, for example system 502 is used when delivering epidural anesthesia, for example during childbirth, during and/or post a surgical procedure, and/or when treating chronic pain.

According to some exemplary embodiments, when a pregnant patient arrives at a hospital for a childbirth, she is connected to epidural anesthesia. In some embodiments, during and/or following the connection of the patient to the epidural anesthesia, she is also connected to the system, for example system 502. In some embodiments, an expert, for example an anesthesiologist attaches at least one array of stimulating electrodes, and at least one sensory electrodes to the patient.

According to some exemplary embodiments, the expert then inserts the patient information into the system. In some embodiments, the expert sets a desired anesthesia distribution to reach for example, a height of the tenth thoracic vertebra (T10) of the spinal cord. In some embodiments, the expert sets an anesthesia administration rate.

According to some exemplary embodiments, while anesthesia is administered, the system monitors the effect of the anesthesia and/or the anesthesia effect distribution, for example by delivering stimulation to the subject, and detecting a signal form the subject body indicating a response of the subject body to the delivered stimulation. In some embodiments, based on the anesthesia effect monitoring and the anesthesia administration parameters, the system determines a pharmacodynamic profile of the patient, and optionally modifies at least one parameter of the anesthesia administration, and/or one or more of the stimulation parameters accordingly. Alternatively, the system optionally delivers one or more suggestions to the user of the system, for example the expert, to modify the anesthesia administration. In some embodiments, the anesthesia administration is optionally modified in order to reach a clinical state in which pain is reduced to a desired level without or with minor, for example tolerable side effects such as tinnitus, metallic taste, numbness in the fingers, motor block, etc.

According to some exemplary embodiments, the user of the system monitors the clinical state of the patient from outside the patient room, for example using a remote device in communication with the system.

According to some exemplary embodiments, the clinical state of the patient and/or of an embryo changes, and there is a need to perform a surgical procedure. In some embodiments, the system delivers a suggestion to the user how to modify the anesthesia administration in view of the surgical procedure. In some embodiments, the system suggests how much of anesthesia to add in order to increase the anesthesia effect distribution, for example from T10 to T4, that is needed for the surgical procedure. In some embodiments, the system suggestions are based on the pharmacodynamic profile of the anesthesia generated for the specific patient.

A potential advantage of generating a personalized pharmacodynamic profile of anesthesia for a patient, is that a more accurate amount of anesthesia can be added to the patient without a risk of developing side effects, for example a low blood pressure due to anesthesia overdose. An additional potential advantage of generating a personalized pharmacodynamics profile of anesthesia for the patient, may be to allow generating a trend or a prediction of a future anesthesia effect in the patient.

### Exemplary Electrodes Position and Arrangement

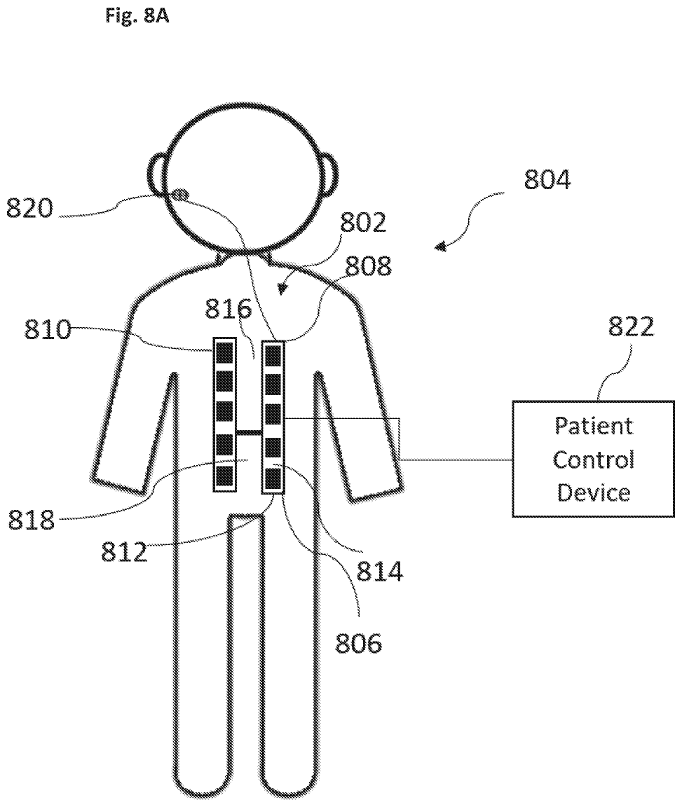

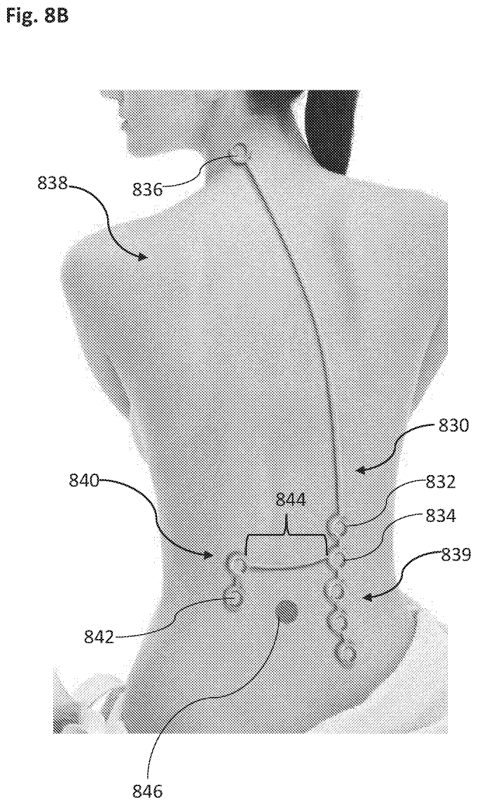

Reference is now made to FIGS. 8A and 8B depicting exemplary arrangements of sensing and stimulating electrodes connected to a control unit of a system for monitoring an anesthesia effect, according to some exemplary embodiments of the invention. In some embodiments, the sensing and the stimulating electrodes are arranged in an electrode patch. In some embodiments, the electrode patch is a disposable electrode patch connectable to the system for monitoring anesthesia, for example anesthesia depth and/or anesthesia height.

According to some exemplary embodiments, the system comprises one or more stimulators, for example one or more stimulating electrodes or stimulation sources. In some embodiments, the one or more stimulating electrodes are attached to a back 802 of a subject 804. In some embodiments, the one or more stimulating electrodes comprise a plurality of stimulating electrodes arranged in an array 806. In some embodiments, the array is shaped as a linear strip of electrodes, or as a panel of horizontally and vertically arranged electrodes. In some embodiments, for example as shown in FIG. 8A, the array comprises two spaced apart arrays, a first array 808 and a second array 810, each is shaped as a strip of electrodes, for example electrodes 812 and 814. In some embodiments, each of the arrays is axially positioned on the back 802 along the spinal cord of the subject, in a different side of the spinal cord 816.

According to some exemplary embodiments, an axial distance between two adjacent electrodes in the array is according to a distance between two adjacent dermatomes, for example to allow position of each electrode of the strip at a different dermatome. In some embodiments, a minimal distance between each array of stimulating electrodes, and an injection site for neuraxial anesthesia.

According to some exemplary embodiments, at least one sensing electrode, for example sensing electrode 820 is attached to a skin surface of the subject, for example behind the car, at a nape region, back, head, and forehead. In some embodiments, each of the stimulating electrodes is separately electrically connected to a control unit of the system, for example to a patient control device. In some embodiments, each of the stimulating electrodes is connected to the control unit by wires or wirelessly. In some embodiments, each array is connected to the control unit by a single cable, a plurality of wires or a single bundle of wires to the control unit 822. Optionally, when using two or more arrays of electrodes, the arrays are interconnected, for example by at least one wire. Optionally, the at least one sensing electrode is connected to one or both of the arrays and/or to the control unit 822.

According to some exemplary embodiments, for example as shown in FIG. 8B, an electrode patch 830 comprises at least one stimulating portion comprises at least one, for example two or more stimulating electrodes, for example stimulating electrodes 832 and 834. In some embodiments, an axial distance between two adjacent electrodes in the stimulating portion of the patch is predetermined according to a distance between two adjacent dermatomes, for example to allow positioning of each electrode of the stimulating portion at a different dermatome. In some embodiments, the electrode patch comprises at least one sensing electrode, for example sensing electrode 836. In some embodiments, the sensing electrode 836 is shaped and sized to be positioned on a head of the subject and/or at a nape region on the back. In some embodiments, a surface of the patch 83, for example a skin contacting surface is configured to be attached to the back of the subject, for example by an adhesive layer, for example glue. In some embodiments, the at least one sensing electrode 836 is coupled to at least one stimulation portion and/or to at least one stimulating portion of the patch.

According to some exemplary embodiments, the electrode patch 830 comprises at least one additional stimulating portion, for example stimulating portion 840, comprising at least one stimulating electrode 842. In some embodiments, a distance 844 between a first stimulating portion 839 and at least one additional stimulating portion 840 is within a range between 5 cm-30 cm, for example between 5 cm-15 cm, between 10 cm-20 cm or any intermediate, shorter or larger distance. In some embodiments, the first stimulating portion 839 and the additional stimulating portion 840 are attached to the back 838 at opposite sides of an anesthesia injection site 846.

According to some exemplary embodiments, each of the stimulating portions include axially separated two or more stimulating electrodes. For example as described with respect to FIG. 8A. A potential advantage of having two stimulating portions configured to stimulate regions of the back at opposite sides of the injection site, may be to allow detection of hemiparesis, a unilateral anesthesia effect on a single side of the body. In hemiparesis, an effect of the subject to a first stimulation of a first side of the body and to a second stimulation of a second side of the body is different, indicating uneven distribution of the anesthesia effect between the two sides of the body.

According to some exemplary embodiments, the electrode patch comprising at least one stimulating electrode and/or at least one sensing electrode, is electrically coupled to a control unit or to the system via a first connector of the patch that is configured to be coupled to a second connector of the control unit or the system. In some embodiments, the first connector has a geometrical shape that fits, for example compliments a geometrical shape of the second connector. In some embodiments, both connectors have a complementing geometrical shape, for example to allow easy connection and/or selectively connection between the electrode patch and the system or control unit of the system.

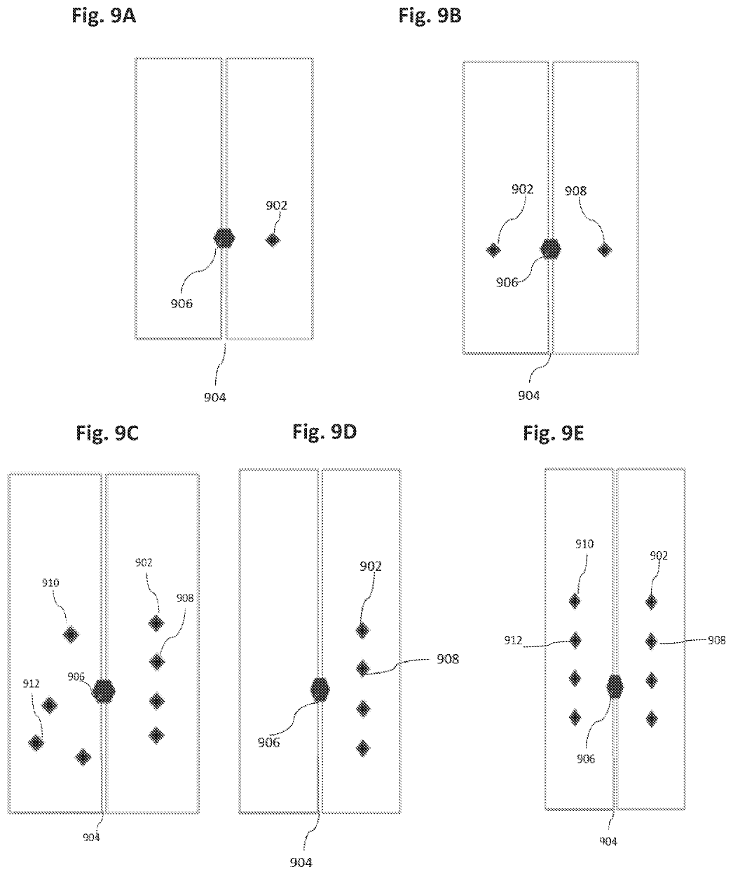

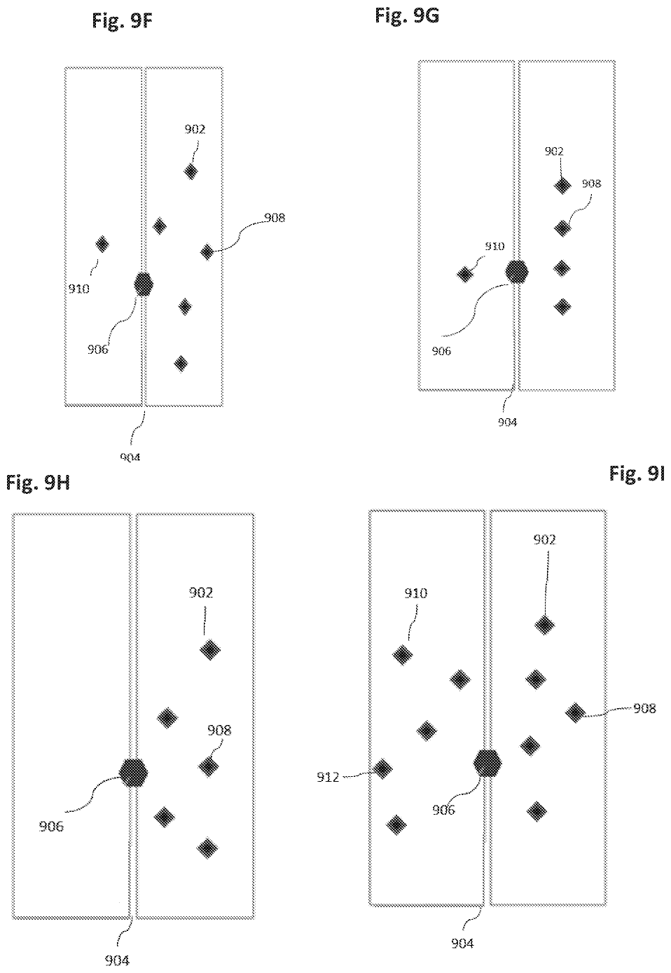

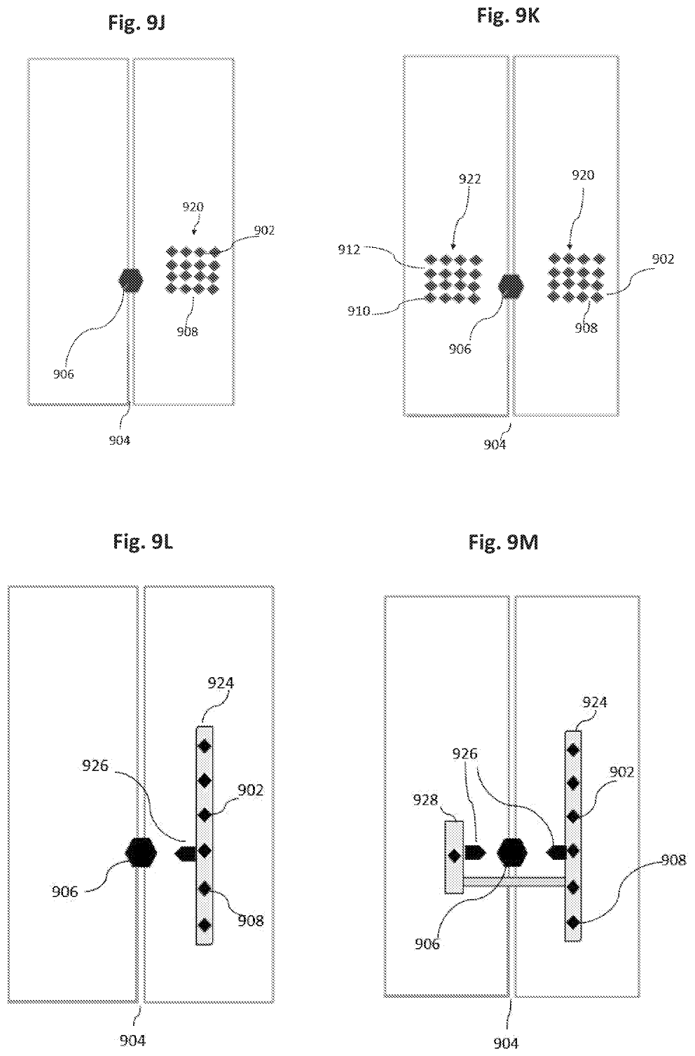

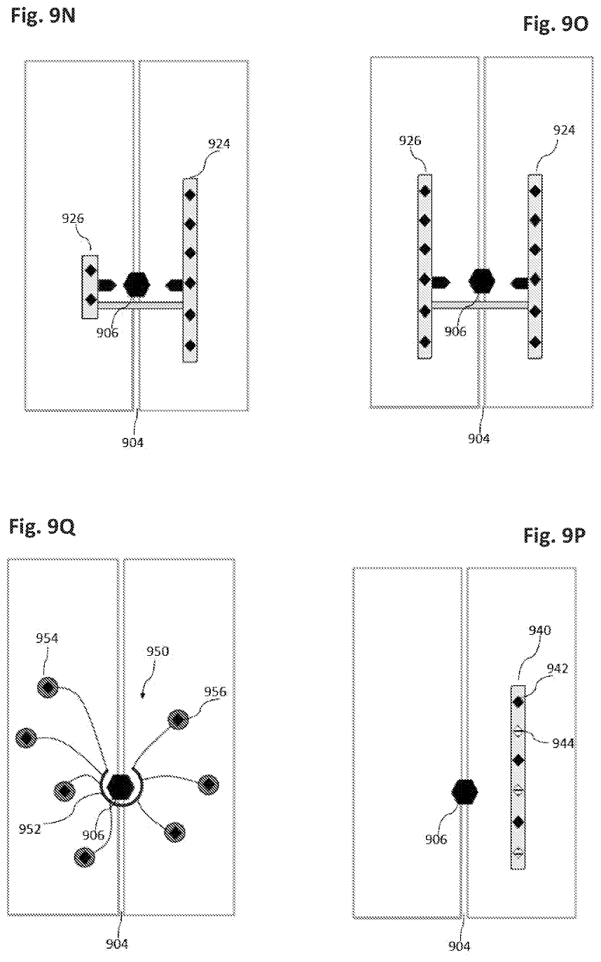

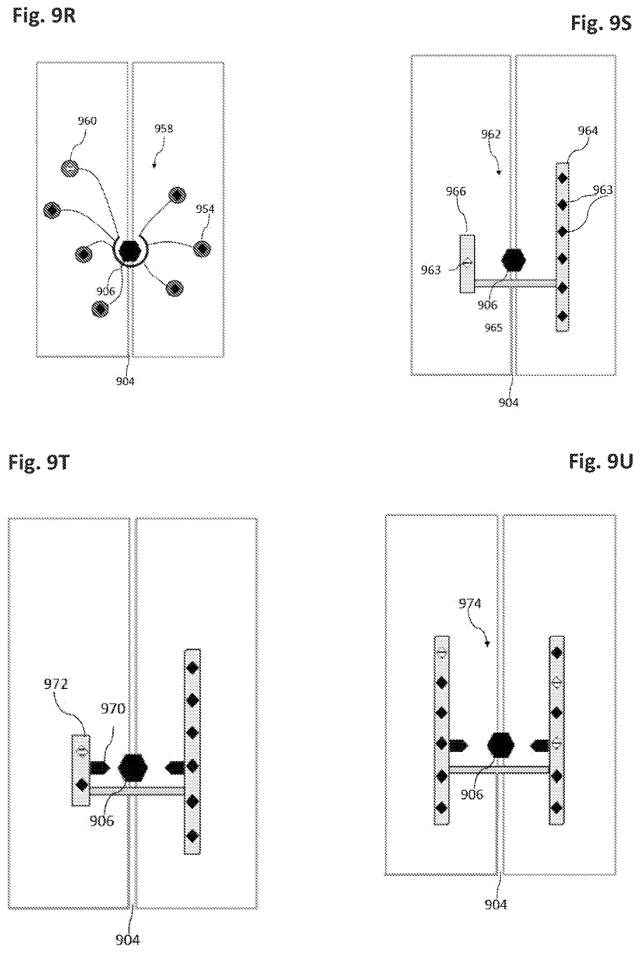

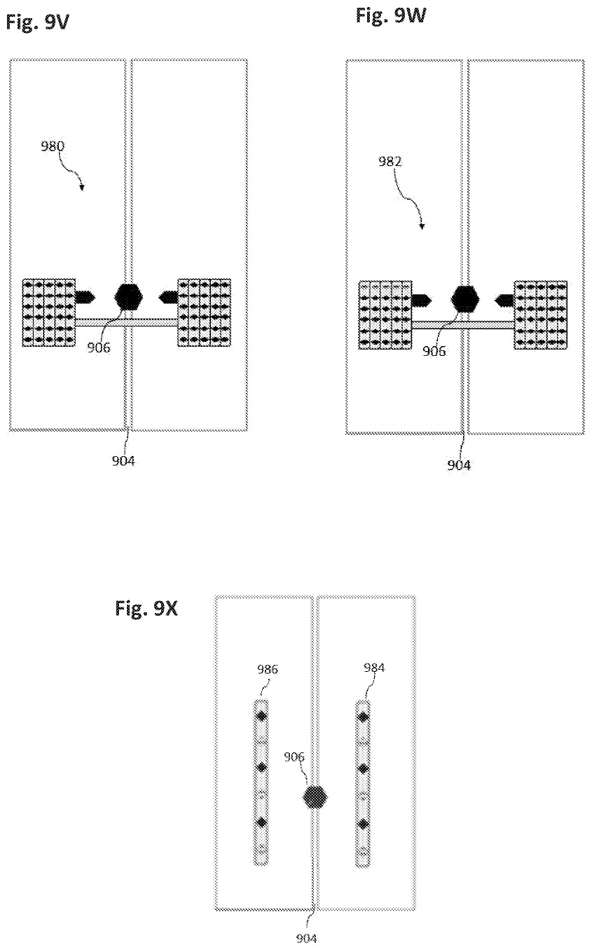

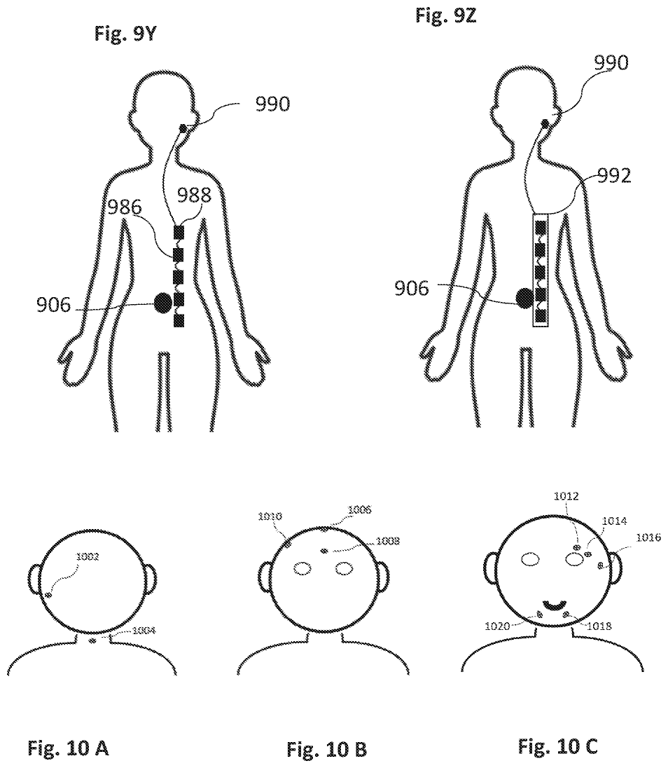

Reference is now made to FIGS. 9A-9Z depicting different arrangements of stimulating and/or sensing electrodes on a body of a subject, according to some exemplary embodiments of the invention.

In some embodiments, for example as shown in FIG. 9A, a single stimulating electrode 902 is positioned, for example attached to a back of a subject. In some embodiments, the stimulating electrode is positioned on a single side of the spinal cord near an anesthetic's injection site 906. Optionally, the stimulating electrode 902 is positioned at a same axial height as the axial height of the injection site 906.

According to some exemplary embodiments, for example as shown in FIG. 9B, two stimulating electrodes, for example stimulating electrodes 902 and 908 are positioned on a back of the subject, each on a different side of the spinal cord 904. Optionally, the electrodes 902 and 908 are positioned at the same axial height as the axial height of the injection site 906.

According to some exemplary embodiments, for example as shown in FIG. 9C, two or more stimulating electrodes, for example electrodes 902 and 908 are axially distributed on a back of the subject at a distance and along the spinal cord 904. In some embodiments, the electrodes 902 and 906 are axially distributed along a longitudinal axis of the body, next to the spinal cord 904 on a first side of the back. Additionally, two or more stimulating electrodes, for example electrodes 910 are randomly distributed on a second side of the back.

According to some exemplary embodiments, for example as shown in FIG. 9D, the stimulating electrodes, for example electrodes 902 and 908 are axially distributed on the first side of the back, with no stimulating electrodes on the second side of the back.

According to some exemplary embodiments, for example as shown in FIG. 9E, a first group of stimulating electrodes, for example electrodes 902 and 908 are axially distributed on a first side of the back, for example as discussed with respect to FIG. 9C. Additionally, a second group of stimulating electrodes, for example electrodes 910 and 912 are axially distributed on a second side of the back. Optionally, electrodes located on a first side of the back, for example electrodes 902 and 908 are positioned in parallel to electrodes on the second side of the back, for example electrodes 910 and 912.

According to some exemplary embodiments, for example as shown in FIG. 9F, a single stimulating electrode 910 is positioned on a first side of the back, and a plurality of stimulating electrodes, for example electrodes 902 and 908 are randomly distributed on a second side of the back. In some embodiments, for example as shown in FIG. 9G, the stimulating electrodes are axially arranged on the second half of the back a long a longitudinal axis of the body. In some embodiments, for example as shown in FIG. 9H, stimulating electrodes are randomly distributed only on one side of the back. Alternatively, for example as shown in FIG. 9I, a plurality of electrodes is randomly distributed on both sides of the back.

According to some exemplary embodiments, for example as shown in FIG. 9J, a plurality of stimulating electrodes is arranged in an array shaped as a panel of electrodes, optionally in two or more lines and two or more rows. In some embodiments, a panel shaped array, for example array 920 including electrodes 902 and 908 is positioned on a single side of the back. Optionally, for example as shown in FIG. 9K, an additional panel 922 including electrodes 910 and 912 is positioned on a second side of the back.

According to some exemplary embodiments, for example as shown in FIGS. 9L-9P, two or more stimulating electrodes, for example electrodes 902 and 908 are axially arranged in an array shaped as a strip. Optionally, a single stimulating electrode is positioned on a strip-shaped array. In some embodiments, for example as shown in FIG. 9O, an array 924 comprises one or more markings, for example marking 926 for aligning the array or at least one electrode of the array with the injection site 906. In some embodiments, a strip shaped array is positioned on a first side of the back. In some embodiments, a second array, for example a strip shaped array 928 is positioned on a second side of the back. Optionally, the array 928 is connected, for example mechanically connected to array 924. In some embodiments, the array 928 is electrically isolated from the array 924.

According to some exemplary embodiments, the array 928 comprises a single stimulation electrode (FIG. 9M), two stimulation electrodes (FIG. 9N), or a plurality of stimulation electrodes (FIG. 9O).

According to some exemplary embodiments, for example as shown in FIG. 9P an array, for example a strip-shaped array 940 comprises at least one stimulating electrode 942 and at least one sensing electrode 944. Optionally, at least one sensing electrode is positioned between two stimulating electrodes, for example as in the array 940.

According to some exemplary embodiments, an electrodes patch, for example electrodes patch 950 shown in FIG. 9Q comprises a central portion 952 and a plurality of electrodes, for example stimulating electrodes 954 connected to the central portion. In some embodiments, at least some of the electrodes are connected to a circumference of the central portion, for example by wires, optionally electrical conducting wires placed within an electrically isolating sheath. In some embodiments, for example as shown in FIG. 9R, electrodes patch 958 comprises at least one sensing electrode 960 and at least one stimulating electrode 954

According to some exemplary embodiments, each electrode comprises an electrode patch, for example a skin patch configured to attach the electrode to a skin surface via a skin-contacting surface of the skin patch. In some embodiments, the skin contacting surface comprises an adhesive layer for attaching the skin patch to the skin surface. In some embodiments, the electrode patch, for example the skin contacting surface, is at least partly flexible and/or soft, to conform to the skin surface anatomy without causing damage to the skin surface. In some embodiments, the central portion 952 comprises an opening which is shaped and sized to surround at least partly the injection site 906. In some embodiments, the central portion 952 is shaped as an arc shaped and sized to surround at least partly the injection site.

Reference is now made to FIGS. 9S to 9W depicting a skin patch comprises two or more interconnected electrode arrays, according to some exemplary embodiments of the invention.

According to some exemplary embodiments, a skin patch comprises a surface configured to attach the skin patch to a skin surface, for example to a skin surface of a back on one or both sides of an injection site. In some embodiments, the skin patch comprises two or more electrode arrays, which are mechanically interconnected to each other. In some embodiments, an electrode array comprises at least one sensing electrode, for example an EMG electrode, an EEG electrode, a neural activity sensing electrode, an ERP sensing electrode, and/or at least one stimulating electrode. Optionally, the electrode array comprises only at least one sensing electrodes or only at least one stimulating electrodes. Optionally, an electrode array comprises a combination of at least one sensing electrode and at least one stimulating electrode. In some embodiments, each array comprises a skin-contacting surface configured to attach the array including the at least one electrode of the array to a skin surface, for example to a skin surface of a back of a patient.

According to some exemplary embodiments, for example as shown in FIG. 9S, a skin patch 962 comprises an electrode array 966 with at least one sensing electrode 963. In some embodiments, the skin patch 962 comprises an additional electrode array 964 which includes a plurality of axially distributed stimulating electrodes 963. In some embodiments, the skin patch 962 comprises at least one connecting portion 965 connecting, for example mechanically interconnecting array 966 and array 964.

According to some exemplary embodiments, for example as shown in FIG. 9T, at least one array of a skin patch, for example array 972 comprises a combination of at least one stimulating electrode and at least one sensing electrode. In some embodiments, one or all of the arrays of a skin patch comprise a marking, for example an alignment marking 970, configured to allow alignment of at least one array and/or at least one electrode of an array with an anatomical location and/or with an injection site in the body, for example injection site 906.

According to some exemplary embodiments, for example as shown in 9U, both or all of the electrode arrays of a skin patch, for example skin patch 974 comprise at least one sensing electrode and at least one stimulating electrode. In some embodiments, an electrode in a first electrode array of a skin patch is axially aligned with an electrode in a second array of the skin patch. In some embodiments, a stimulating electrode in the first electrode array is axially aligned with a stimulating electrode of the second electrode array or with a sensing electrode of the second electrode array.

According to some exemplary embodiments, a skin patch comprises to or more electrode arrays which includes electrodes arranged in two or more rows, for example as shown in FIG. 9V depicting skin patch 980. In some embodiments, the electrodes are axially distributed in each row, in fixed or varying distance between two adjacent electrodes. In some embodiments, for example as shown in FIG. 9V, each electrode array of the two or more electrode arrays include a similar number of electrodes. Optionally, an electrode array is axially aligned with a second electrode array, for example on opposite sides of an injection site 906. In some embodiments, for example as shown in FIG. 9W, one or more electrodes of a first electrode array are not axially aligned with electrodes of a second electrode array. Optionally, each electrode array includes a different number of electrodes, optionally arranged in a different pattern relative to at least one different electrode array of the same electrode patch. In some embodiments, electrodes of an electrode array comprise at least one stimulation electrode and/or at least one sensing electrode.

According to some exemplary embodiments, for example as shown in FIG. 9X, the system comprises electrodes for example sensing and/or stimulating electrodes arranged in two or more separate array, for example strip arrays or panel array.

According to some exemplary embodiments, for example as shown in FIG. 9Y, at least one, or two or more stimulating electrodes, for example electrodes 986 and 988 are connected to each other, and to at least one sensing electrodes 990, for example an EEG electrode located on a head or nape of the subject. In some embodiments, the two stimulating electrodes and the at least one sensing electrodes are axially connected to each other, for example in a column. In some embodiments, for example as shown in FIG. 9Z, the stimulating electrodes are arranged in an array 992 attached to a back of the patient. In some embodiments, the array 992 is connected to the at least one sensing electrode 990, for example by wire. In some embodiments, each stimulating electrode and the at least one sensing electrode is separately electrically connected to a control unit of the system.

### Exemplary Sensing Electrode Position

According to some exemplary embodiments, the sensing electrodes of the system comprise ERP-sensing electrodes, neural activity sensing electrodes, EEG electrodes and/or EMG electrodes. In some embodiments, the sensing electrodes, for example the EEG electrodes are configured to record ERP signals, for example SSEP signals, for example D-SSEP signals. In some embodiments, the sensing electrodes, for example the EMG electrodes are configured to record EMG signals from one or muscles of the patient.

According to some exemplary embodiments, for example as shown in FIG. 10A, EEG electrodes, for example electrodes 1002 and 1004 are positioned at sub-cortical locations, for example behind the car of a patient (electrode 1002) and/or on a nape of the patient (electrode 1004), respectively. Alternatively or additionally, for example as shown in FIG. 10B, the electrodes, for example EEG electrodes 1006, 1008 and 1010 are positioned on a scalp of a subject at cortical locations. In some embodiments, cortical locations means locations on a scalp of a subject above cortical locations.

According to some exemplary embodiments, for example as shown in FIG. 10C, at least some of the sensing electrodes. For example, electrodes 1012, 1014, 1016, 1018, and 1020, are positioned on a head of a subject above face muscles, for example upper eyelid muscle, temporalis muscle, and mentalis muscle.

According to some exemplary embodiments, a combination of different types of sensing electrodes is used, for example a combination of EEG and EMG electrodes.

Exemplary Neuropathy Detection and/or Evaluation

According to some exemplary embodiments, the system described herein is used for detection and/or evaluation of neuropathy, for example peripheral neuropathy and diabetic neuropathy. As used herein, peripheral neuropathy a general term describing disease affecting the peripheral nerves, meaning nerves beyond the brain and spinal cord. As used herein diabetic neuropathy is a type of nerve damage that can occur during diabetes. For example, diabetic foot.

According to some exemplary embodiments, in order to detect and/or to evaluate neuropathy, at least one stimulating electrode is attached in proximity to a tissue expected to be affected by neuropathy. In some embodiments, the system monitors the response of the tissue to a stimulation by the at least one stimulating electrode, by recording signals from at least one sensing electrode, for example an EEG electrode, an EMG electrode or any other electrode capable of recording neural activity or neural transmission, attached to a skin surface, for example attached to a back, nape, forehead, head, above sub-cortical regions and/or above cortical regions. In some embodiments, changes in the recorded signal in response to a stimulation over time indicates a neuropathic state of the stimulated tissue. In some embodiments, reduction in the tissue responsiveness to the stimulation signal, as indicated by the recorded signal, indicates a neuropathic state of the stimulated tissue.

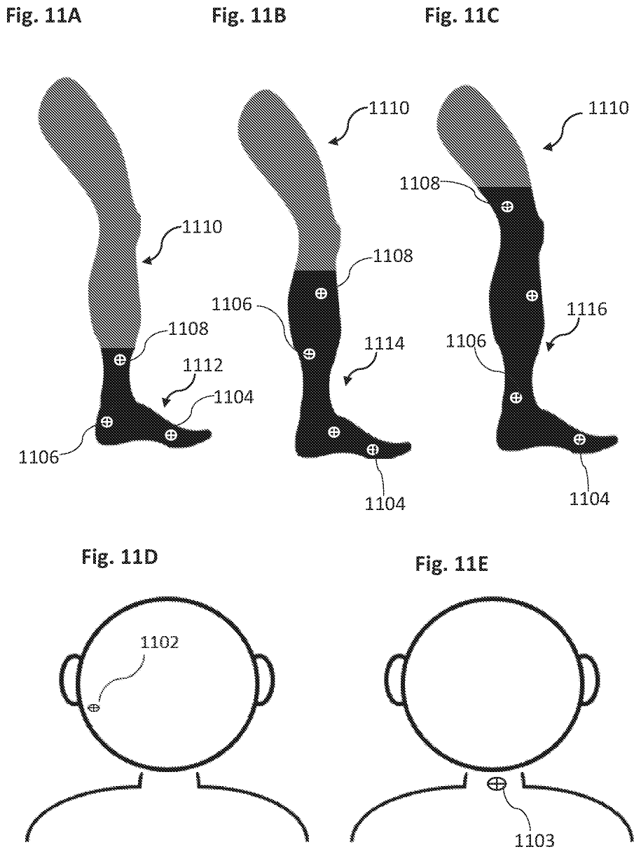

Reference is now made to FIGS. 11A-11D depicting using the system for identifying diabetic neuropathy in a diabetic leg, according to some exemplary embodiments of the invention.

According to some exemplary embodiments, at least one stimulating electrode, for example a plurality of stimulating electrode is positioned along a leg of a patient. In some embodiments, stimulation is provided through each stimulating electrode while recording neuronal electrical activity by at least one sensing electrode, for example at least one EEG electrode located optionally behind an car, on a back and/or nape and/or above sub-cortical or cortical regions. In some embodiments, for example as shown in FIG. 11D, the at least one sensing electrode 1102 is positioned behind the car. Alternatively or additionally, for example as shown in FIG. 11E, at least one sensing electrode 1103 is positioned at a nape region.

According to some exemplary embodiments, stimulation is first provided through at least one distal electrode, for example an electrode located close to the leg fingers, and then in a sequence through more proximal stimulation electrodes, for example electrodes located closer to a knee. In some embodiments, signals are recorded by at the at least one sensing electrode after each stimulation. In some embodiments, diabetic neuropathy is detected when a change in at least one parameter of the recorded signal is detected, for example compared to reference measurements, compared to baseline measurements and/or compared to signals recorded from a different tissue region. Optionally, the detected change is a change in the at least one signal parameter when comparing signals recorded from two different regions in response to a similar stimulation, for example to a stimulation having similar parameter values. In some embodiments, the stimulation parameters comprise pulse intensity, frequency, and/or duration. In some embodiments, the detected change comprises a detected degradation in the recorded signal, for example when comparing signals recorded from two different regions. In some embodiments, the degradation in the recorded signal comprises at least one of, degradation in signal quality, degradation in the length of the signal, degradation in intensity of the signal, and changes in signal frequency.

According to some exemplary embodiments, for example as shown in FIGS. 11A-11C, two or more stimulating electrodes, for example electrodes 1104, 1106, 1108 are positioned on a leg 1110 of a patient as part of a sock, for example sock 1112 or a wearable band or any wearable elastic material.

According to some exemplary embodiments, in order to monitor an advancement of the neuropathy towards more proximal regions of the leg, one or more electrodes are positioned at more proximal locations. Optionally, longer socks or wearables elastic materials, for example socks 1114 and 1116 are used with additional stimulating electrodes at more proximal locations along the leg 1110.

### Exemplary Monitoring Local Anesthesia Effect

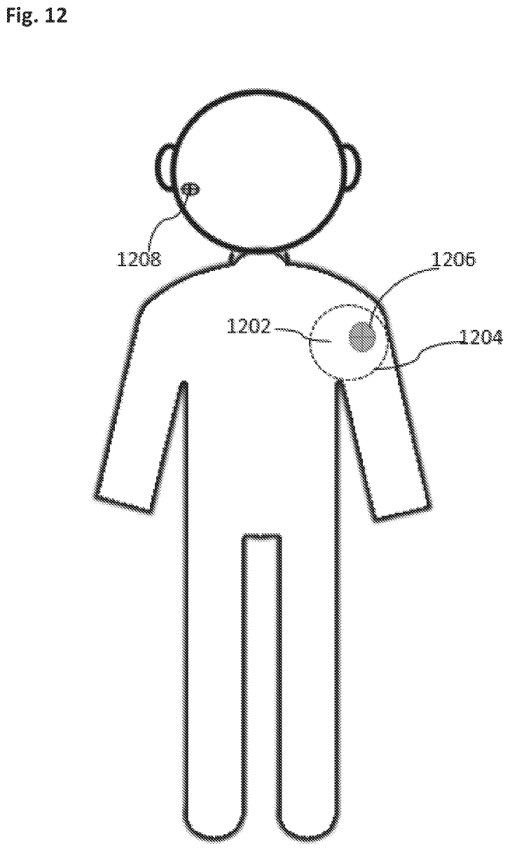

Reference is now made to FIG. 12 depicting monitoring local anesthesia effect, according to some exemplary embodiments of the invention.

According to some exemplary embodiments, a system described herein is used to monitor an effect of local anesthesia on a tissue over time. As used herein, local anesthesia means an anesthesia that is intended to affect a small region of a body surrounding an injection site of the local anesthetic, for example a region which is smaller than 20 cm2, for example smaller than 15 cm2, smaller than 10 cm2, smaller than 5 cm2 or any intermediate, smaller or larger range of value around the injection site of the local anesthetic.

According to some exemplary embodiments, for example as shown in FIG. 12. A local anesthetic is injected at injection site 1202. In some embodiments, the local anesthetic is injected with a dose or concentration sufficient to anesthetize target region 1204. In some embodiments, at least one stimulating electrode, for example electrode 1206 is positioned within the target region 1204. In some embodiments, at least one sensing electrode, for example sensing electrode 1208 is positioned in at least one of, back, head nape, above sub-cortical or cortical regions, and is configured to record electrical signals following stimulation of the target region 1204 by the at least one stimulating electrode 1206. In some embodiments, the at least one sensing electrode is positioned in at least one body region and is configured to record at least one of, neural activity indicating signals, ERP signals, for example SSEP, D-SSEP, or EMG.

According to some exemplary embodiments, the effect of the local anesthetic on the target region 1204 is determined by measuring a change in the recorded signal after injection of anesthetic relative to a signal measured prior to anesthetic, relative to reference value, relative to a baseline value, relative to a signal measured from a non-anesthetized tissue, optionally in response to stimulation with similar parameter values. Optionally, the change in signal indicates reduction in neural transmission in the target region following local anesthetic injection.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1.-70. (canceled)

71. A method for determining an effect of anaesthesia in a subject, comprising:
stimulating a body part of a subject at one or more stimulation sites, wherein said body part is under local or regional anaesthesia and wherein said stimulation is lower than a pain sensation threshold of the subject;
measuring a neurological response caused by said stimulation using an electrode configured to measure event-related potentials (ERP), wherein said electrode is positioned on the head or nape of the subject,
applying a trained machine learning algorithm on the measured neurological response to determine an effect of said local or regional anaesthesia.

72. The method according to claim 71, wherein said measuring comprises measuring said response in up to 300 milliseconds following said stimulation.

73. The method according to claim 71, wherein determining said regional anaesthesia effect comprises determining a depth of said local or regional anaesthesia.

74. The method according to claim 71, wherein the anaesthesia is regional anaesthesia and wherein the method further comprises repeating said stimulating at two or more axially spaced-apart stimulation sites, and wherein said machine learning algorithm is further configured to assess an axial distribution of said regional anaesthesia.

75. The method according to claim 74, wherein said machine learning algorithm is further configured to output a prediction regarding the effect of the regional anaesthesia effect, based on the axial distribution.

76. The method of claim 74, wherein the body part is a back of the subject.

77. The method according to claim 74, wherein said one or more stimulation sites comprise at least one stimulation site in one or more dermatomes located between S5 to T2 dermatomes.

78. The method according to claim 71, wherein said stimulating comprises initially stimulating said body part before administering said local or regional anaesthesia, and wherein a neurological response to the initial stimulation is further provided as an input to the machine learning algorithm.

79. The method according to claim 71, wherein said stimulating comprises delivering an electric field to said body part using a stimulating electrode, wherein said delivered electric field has an intensity value in a range between 0.5-40 mA and/or a frequency value in a range between 1-4000 Hz.

80. The method according to claim 71, wherein said stimulating comprises delivering a temperature stimuli.

81. The method according to claim 71, further comprising generating an alert signal if the effect of the local or regional anaesthesia deviates or predicted to deviate from a planned effect of the anaesthesia.

82. The method according to claim 71, wherein said machine learning algorithm is further configured to identify hemiparesis in said subject, based on said determined regional anaesthesia effect.

83. The method according to claim 71, wherein said machine learning algorithm is further configured to output a pharmacodynamic profile of one or more anaesthetic compounds used for said anaesthesia in said subject, a trend of said anaesthesia effect and/or a prediction of said anaesthesia effect, based on said determined anaesthesia effect

84. A system for monitoring an effect of anaesthesia in a subject, the system comprising:
at least one stimulator configured to deliver stimulation to at least one stimulation site on a body part of the subject, wherein said stimulation is lower than a pain sensation threshold of the subject;
at least one event-related potentials (ERP) electrode configured to measure the neurological response to the stimulation, wherein the at least one ERP electrode is configured to be positioned on a head or nape of the subject;
a memory; and
a control circuitry operationally connected to said memory, said at least one stimulator and said at least one ERP electrode;
wherein said control circuitry is configured to:
activate said at least one stimulator to deliver a stimulation to said at least one stimulation site, according to stimulation parameters values stored in said memory;
receive at least one signal from said at least one ERP electrode measured in response to said stimulation; and
to apply a trained machine learning algorithm on the received signal to thereby determine the anaesthesia effect on said body part.

85. The system of claim 84, wherein the at least one ERP electrode is configured to measure the neurological response in up to 300 ms after the stimulation.

86. The system according to claim 84, wherein determining said anaesthesia effect comprises determining an axial distribution of the anaesthesia and/or a depth of the anaesthesia.

87. The system according to claim 84, wherein said at least one stimulator comprises at least one stimulating electrode shaped and sized to be positioned at said at least one stimulation site, wherein said system further comprises at least one pulse generator functionally connected to said at least one stimulating electrode, and wherein said control circuitry is configured to:
activate said pulse generator to generate and deliver an electric field to said at least one stimulating electrode, wherein said electric field is generated according to electric field parameter values stored in said memory.

88. The system according to claim 84, wherein said control circuitry determines an effect of said anaesthesia by activating said at least one pulse generator to generate and deliver two or more electric fields separated in time and in a stimulation location, by measuring a neurological response to the two or more electric fields, and by determining a relation between a first measured neurological response to a first electric field delivery, and a second neurological response to a second electric field delivery.

89. The system according to claim 88, wherein said control circuitry activates said pulse generator to generate and deliver two consecutive electric fields with an interval between the two consecutive electric field which is higher than 180 milliseconds.

90. The system according to claim 89, wherein an intensity of said generated electric field is in a range between 0.5 mA-40 mA and/or wherein a frequency of said generated electric field is in a range between 0.1 Hz-4000 Hz.
